(19) United States
(12) Patent Application Publication
(10) Pub. No.: US 2025/0197482 A1
(43) Pub. Date: Jun. 19, 2025

(54) BISPECIFIC ANTIBODIES TO HIV-1 ENV AND THEIR USE

(71) Applicant: The United States of America, as represented by the Secretary, Department of Health and Human, Bethesda, MD (US)

(72) Inventors: Peter Kwong, Washington, DC (US); Baoshan Zhang, Bethesda, MD (US); Leland Damron, Bethesda, MD (US); Tatsiana Bylund, Cedar Point, NC (US); Amarendra Pegu, Rockville, MD (US); Eun Sung Yang, Bethesda, MD (US); Jason Gorman, Silver Spring, MD (US); Young Do Kwon, Rockville, MD (US); Yongping Yang, Potomac, MD (US); Nicole Doria-Rose, Garrett Park, MD (US)

(73) Assignee: The United States of America, as represented by the Secretary, Department of Health and Human, Bethesda, MD (US)

(21) Appl. No.: 18/848,883

(22) PCT Filed: Mar. 27, 2023
(86) PCT No.: PCT/US2023/064996

§ 371 Date: Sep. 19, 2024

Related U.S. Application Data
(60) Provisional application No. 63/324,037, filed on Mar. 26, 2022.

(51) Int. Cl. C07K 16/10; A61P 31/18; G01N 33/569

(52) U.S. Cl. CPC C07K 16/1063; A61P 31/18; G01N 33/56988; C07K 2317/31; C07K 2317/52; C07K 2317/565; C07K 2317/567; C07K 2317/569; C07K 2317/72; C07K 2317/76; G01N 2333/162

(57) ABSTRACT

Bispecific antibodies that specifically bind to HIV-1 Env and neutralize HIV-1 are disclosed. Nucleic acids encoding these bispecific antibodies, vectors and host cells are also provided. In addition, the use of these bispecific antibodies, nucleic acid molecules, and vectors to prevent and/or treat an HIV-1 infection is disclosed.

### CROSS REFERENCE TO RELATED APPLICATION

This application is the U.S. National Stage of International Application No. PCT/US2023/064996, filed Mar. 27, 2023, which was published in English under PCT Article 21(2), which in turn claims priority to U.S. Provisional Application No. 63/324,037, filed Mar. 26, 2022. The provisional application is incorporated by reference in its entirety.

### SEQUENCE LISTING

The nucleic and amino acid sequences listed in the accompanying sequence listing are shown using standard letter abbreviations for nucleotide bases, and single letter code for amino acids, as defined in 37 C.F.R. 1.822. Only one strand of each nucleic acid sequence is shown, but the complementary strand is understood as included by any reference to the displayed strand. The Sequence Listing is submitted as an XML file in the form of the file named “4239-108058-07_Sequence” (˜86,016 bytes), which was created on Mar. 27, 2023, which is incorporated by reference herein.

### FIELD OF THE DISCLOSURE

This relates to bispecific antibodies that specifically bind to HIV-1 Env and their use, for example, in methods of treating a subject with HIV-1 infection.

### BACKGROUND

Human Immunodeficiency Virus type 1 (HIV-1) infection, and the resulting Acquired Immunodeficiency Syndrome (AIDS), remain threats to global public health, despite extensive efforts to develop anti-HIV-1 therapeutic agents.

An enveloped virus, HIV-1 hides from humoral recognition behind a wide array of protective mechanisms. The major HIV-1 envelope protein (HIV-1 Env) is a glycoprotein of approximately 160 kD (gp160). During infection, proteases of the host cell cleave gp160 into gp120 and gp41. Together gp120 and gp41 make up the HIV-1 envelope spike, which interacts with the host-cell receptor CD4 to facilitate virus infection, and is a target for neutralizing antibodies.

Prior studies identified the VRC26.25 antibody, which specifically binds to the V1-V2 region of HIV-1 Env, and the J3 V_{H}H, which specifically binds to the CD4-binding site of HIV-1 Env, as potent HIV-1 neutralizing antibodies with suboptimal breadth. However, there is a need to develop additional neutralizing antibodies for HIV-1 with improved neutralization profiles for clinical treatment or prevention of HIV.

### SUMMARY

Disclosed herein are bispecific antibodies comprising a first binding domain that specifically binds to a V1-V2 region of HIV-1 Env and a second binding domain that specifically binds to a CD4 binding site on HIV-1 Env. The first binding domain comprises an antibody comprising a heavy chain variable region (V_{H}) comprising a heavy chain complementarity determining region (HCDR)1, a HCDR2, and a HCDR3 comprising amino acid sequences set forth as SEQ ID NOs: 2, 3, and 4, respectively, a light chain variable region (V_{L}), and a constant domain. The second binding domain comprises a VHH comprising a HCDR1, a HCDR2, and a HCDR3 comprising amino acid sequences set forth as SEQ ID NOs: 10, 11, and 12, respectively. The C-terminus of the V_{H}H is fused to the N terminus of the V_{L} by a peptide linker. The first and second binding domains can simultaneously bind to a single HIV-1 Env trimer, to synergistically neutralize HIV-1. Data provided herein shows that the combination of the first and second binding domains in the bispecific antibody provides a synergistic and beneficial result for HIV-1 neutralization.

Also disclosed are compositions including the antibodies as well as related nucleic acid molecules and expression vectors.

The disclosed bispecific antibodies potently neutralize HIV-1 in an accepted in vitro model of HIV-1 infection. Accordingly, a method is disclosed for inhibiting an HIV-1 infection in a subject, comprising administering an amount of one or more of the disclosed bispecific antibodies or nucleic acid molecules, vectors, or compositions, to the subject, effective to prevent or treat HIV-1 infection in the subject. In several implementations, the subject is at risk of or has an HIV-1 infection.

The bispecific antibodies, nucleic acid molecules, vectors, and compositions disclosed herein can be used for a variety of additional purposes, such as for detecting an HIV-1 infection or diagnosing HIV-1 infection in a subject, or detecting HIV-1 in a sample.

The foregoing and other features and advantages of this disclosure will become more apparent from the following detailed description of several implementations which proceeds with reference to the accompanying figures.

### BRIEF DESCRIPTION OF THE FIGURES

FIGS. 1A-1D. Design of HIV-1 bispecific antibodies attaching nanobodies or scFvs to the light chain of the super potent V2-apex-directed antibody CAP256-VRC26.25. (FIG. 1A) Structure of the antigen-binding fragment (Fab) of antibody CAP256-VRC26.25 in complex with a prefusion-closed HIV-1 Env trimer showing an unencumbered light chain allowing its linkage to other HIV-1 trimer-binding components. The resultant bispecific antibody enables a synergistic binding and an enhanced neutralization. (FIG. 1B) Schematic of V1V2 antibody with additional binding component genetically fused to the light chain N terminus. (FIG. 1C) Schematic of VRC26.25 antibody with J3VHH genetically fused to the VRC26.25 light chain N terminus. (FIG. 1D) Light chain variant expression constructs. Sequence information of these variants are listed in supplementary figures. Upper construct shows light chain variants linked with nanobody. Bottom construct shows light chain variants linked to single chain Fv.

FIGS. 2A-2D. Evaluation of antibody variants and select of variants with improved neutralizing breadth. (FIG. 2A) Screening of designed antibody variants for binding to trimers from CAP256V2LS-resistant virus strains and neutralization on a CAP256V2LS-resistant virus. Darker shading indicates better binding or neutralization. (FIG. 2B) Neutralization IC₅₀ of CAP256V2LS nanobody variants and scFv variants on small virus panels. (FIG. 2C) Neutralization IC₅₀ of PGDM1400 antibody variants on a 5-virus panel. (FIG. 2D) Neutralization IC₈₀ of selected CAP256V2LS and PGDM1400 antibody variants on a 30-virus panel. Geometric mean IC₈₀ values (μg/ml) are indicated at the bottom of each antibody column.

FIGS. 3A-3E. Structure of CAP256V2LS-J3-3 Fab in complex with BG505 DS-SOSIP.664 confirms avidity and stoichiometry. (FIG. 3A) Cryo-EM density is shown highlighting the linker between CAP256V2LS and J3 at 3.2 Å resolution and contour of 0.22 where density for the flexible linker was visible. (FIG. 3B) Cryo-EM density is shown with lower contour to illustrate the signal observed corresponding to unbound CAP256V2LS (FIG. 3C, left), cryo-EM density is shown with higher contour revealing greater detail of higher resolution signal (FIG. 3C, right). Corresponding atomic models are shown in cartoon format with glycans shown as spheres. (FIG. 3D) Details of binding for V CAP256V2LS and J3 from the bivalent complex structure are overlayed with the structures obtained from individual components. (FIG. 3E) The CDR H3 of CAP256V2LS aligns closely while the main body of the Fab shifts as much as ˜16 Å.

FIGS. 4A-4G. Improved pharmacokinetics of CAP256.J3LS achieved by altering surface charge of J3. (FIG. 4A) In vivo half-life of CAP256—variants assessed in a human FcRn knock-in mouse model. (FIG. 4B) Sequence of J3 with Arg and Lys residues highlighted. (FIG. 4C) Accessible surface area of Arg and Lys residues. Residues above the dotted line were altered mutationally to reduce electropositivity. (FIG. 4D) Arg and Lys residues that were selected for mutations were shown in the structure of J3 in complex with gp120 (PDB ID: 7RI1). (FIG. 4E-4F) Neutralization IC₈₀ fold change, heparin chromatography retention volume, and autoreactivity of CAP256.J3LS variants. (FIG. 4G) In vivo half-life of CAP256.J3LS variants.

FIGS. 5A-5C. Neutralization breath and potency of CAP256.J3LS antibody. (FIG. 5A) Neutralization IC₈₀ of CAP256.J3LS on a 208 global virus panel in comparison with parental antibodies and select HIV-1 antibodies in clinical development. (FIG. 5B) Neutralization IC₈₀ of CAP256.J3LS on a 100 Acute-Early Clade C virus panel. (FIG. 5C) Breath-IC₈₀ curves of CAP256.J3LS in comparison with parental antibodies and select HIV-1 antibodies in clinical development.

FIG. 6. Screening of designed antibody variants for binding to trimers from CAP256V2LS-resistant virus strains and neutralization on a CAP256V2LS-resistant strains. Antibody heavy and light chain identify is indicated in each row for one antibody variant. Each row represents the results of binding or neutralization. Values of ELISA binding to each trimer protein and percentage neutralization against two viruses are listed.

FIGS. 7A-7B. Neutralization IC₈₀ of selected antibody variants on a 30-virus panel. Geometric mean IC₈₀ values (μg/ml) are indicated at the bottom of each antibody column.

FIGS. 8A-8E. Cryo-EM details of VRC26.25-J3 in complex with BG505 DS-SOSIP.664. (FIG. 8A) Representative micrograph with scale bar at 200 nm. (FIG. 8B) Representative 2D class averages are shown. (FIG. 8C) The orientations of all particles used in the final refinement are shown as a heatmap. (FIG. 8D) The gold-standard fourier shell correlation resulted in a resolution of 3.90 Å using non-uniform refinement with C1 symmetry. (FIG. 8E) The local resolution of the full map is shown generated through cryoSPARC using an FSC cutoff of 0.5. Two contour levels are shown at 0.22 and 0.4 as in FIGS. 3A and 3C.

FIGS. 9A-9B. Neutralization data of CAP256LS-J3-3 charge variants. Geometric mean IC₅₀ and IC₈₀ values (μg/ml) are indicated at the bottom of each antibody column.

FIG. 10. Autoreactivity data of CAP256LS-J3-3 charge variants.

FIGS. 11A-11C. Characterization of CAP256.J3LS antibody. (FIG. 11A) Size-exclusion chromatography profile. (FIG. 11B) HEp-2 cell staining assay against CAP256V2LS variants. Lower right corner numbers at 25 μg/ml are autoreactivity scores for antibody variants. (FIG. 11C) Summary of anti-cardiolipin ELISA.

FIG. 12. Isothermal titration calorimetry measurement of CAP256.J3LS. Isothermal titration calorimetry of HIV-1 trimer with CAP256.J3LS and parental antibodies at pH 7.4 and 37° C. The binding thermodynamic parameters including the affinities and stoichiometries are shown for each antibody.

FIGS. 13A-13C. Neutralization fingerprinting analysis. (FIG. 13A) Fingerprinting analysis reveals improved bi-specific CAP256.J3LS antibodies cluster with V1V2 apex antibodies. (FIG. 13B) Histogram of fold decrease in IC₅₀ values for each antibody/pseudovirus pair comparing CAP256 variants to CAP256-VRC26.25 (WT). Pairs where IC₅₀ exceeded 50 μg/ml not shown. Dotted vertical line indicates no change in potency. (FIG. 13C) Histogram of fold decrease in IC₅₀ values for each antibody/pseudovirus pair comparing CAP256 variants to J3 (WT). Pairs where IC₅₀ exceeded 50 μg/ml not shown. Dotted vertical line indicates no change in potency.

FIG. 14. Manufacturability and biophysical risk assessment (MBRA).

SEQUENCES

SEQ ID NO: 1 is the amino acid sequence of the CAP256V2LS VH.
QVQLVESGGGVVQPGTSLRLSCAASQFRFDGYGMHWVRQAPGKGLEWVASISHDGIKKYHAEKVWGRFTISR
DNSKNTLYLQMNSLRPEDTALYYCAKDLREDECEEWWSDYYDFGAQLPCAKSRGGLVGIADNWGQGTMVTVS
S
SEQ ID NOs: 2-4 are the CDR sequences of the CAP256V2LS VH.
SEQ ID NO: 2-HCDR1, GYGMH
SEQ ID NO: 3-HCDR2, SISHDGIKKYHAEKVW
SEQ ID NO: 4-HCDR3, DLREDECEEWWSDYYDFGAQLPCAKSRGGLVGIADN
SEQ ID NO: 5 is the amino acid sequence of the CAP256V2LS VL.
QSVLTQPPSVSAAPGQKVTISCSGNTSNIGNNFVSWYQQRPGRAPQLLIYETDKRPSGIPDRFSASKSGTSG
TLAITGLQTGDEADYYCATWAASLSSARVFGTGTQVIVL
SEQ ID NOs: 6-8 are the CDR sequences of the CAP256V2LS VL.
SEQ ID NO: 6-LCDR1, SGNTSNIGNNFVS
SEQ ID NO: 7-LCDR2, ETDKRPS
SEQ ID NO: 8-LCDR3, ATWAASLSSARV
SEQ ID NO: 9 is the amino acid sequence of the J3 V_{H}H.
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSS
SEQ ID NOs: 10-12 are the CDR sequences of the J3 V_{H}H.
SEQ ID NO: 10-HCDR1, QYAMA
SEQ ID NO: 11-HCDR2, GMGAVPHYGEFVKG
SEQ ID NO: 12-HCDR3, SKSTYISYNSNGYDY
SEQ ID NO: 13 is the amino acid sequence of the CAP256V2LS-J3-3 VHH and VL portions (no
charge variations).
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTL
SEQ ID NO: 14 is the amino acid sequence of the CAP256.J3LS HC (VRC-6522).
QVQLVESGGGVVQPGTSLRLSCAASQFREDGYGMHWVRQAPGKGLEWVASISHDGIKKYHAEKVWGRFTISR
DNSKNTLYLQMNSLRPEDTALYYCAKDLREDECEEWWSDYYDFGAQLPCAKSRGGLVGIADNWGQGTMVTVS
SASTKGPSVFPLAPSSKSTSGGTAALGCLVKDYFPEPVTVSWNSGALTSGVHTFPAVLQSSGLYSLSSVVTV
PSSSLGTQTYICNVNHKPSNTKVDKKVEPKSCDKTHTCPPCPAPELLGGPSVFLFPPKPKDTLMISRTPEVT
CVVVDVSHEDPEVKFNWYVDGVEVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWESNGQPENNYKTTPPVLDSDGSF
FLYSKLTVDKSRWQQGNVFSCSVLHEALHSHYTQKSLSLSPGK
SEQ ID NO: 15 is the amino acid sequence of the CAP256V2LS-J3-3 LC (including J3, no charge
variations).
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 16 is the amino acid sequence of a peptide linker.
ggsggggsggggsgg
SEQ ID NO: 17 is the amino acid sequence of the CAP256.J3LS LC (R19E/K83E/R105Q)
(including modified J3 VRC-7726).
EVQLVESGGGLVQAGGFLeLSCELRGSIFNQYAMAWFRQAPGKEREFVAGMGAVPHYGEFVKGRFTISRDNA
KSTVYLQMSSLePEDTAIYFCARSKSTYISYNSNGYDYWGqGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 18 is the amino acid sequence of the CAP256V2LS-J3-3.C1 LC
(R19E/K75E/R105Q) (including modified J3).
EVQLVESGGGLVQAGGFLeLSCELRGSIFNQYAMAWFRQAPGKEREFVAGMGAVPHYGEFVKGRFTISRDNA
eSTVYLQMSSLKPEDTAIYFCARSKSTYISYNSNGYDYWGqGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 19 is the amino acid sequence of the CAP256V2LS-J3-3.C2 LC
(R19E/K83E/R105N) (including modified J3).
EVQLVESGGGLVQAGGFLeLSCELRGSIFNQYAMAWFRQAPGKEREFVAGMGAVPHYGEFVKGRFTISRDNA
KSTVYLQMSSLePEDTAIYFCARSKSTYISYNSNGYDYWGnGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 20 is the amino acid sequence of the CAP256V2LS-J3-3.C3 LC
(R19E/K75E/K83E/R105Q) (including modified J3).
EVQLVESGGGLVQAGGFLeLSCELRGSIFNQYAMAWFRQAPGKEREFVAGMGAVPHYGEFVKGRFTISRDNA
eSTVYLQMSSLePEDTAIYFCARSKSTYISYNSNGYDYWGqGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 21 is the amino acid sequence of the CAP256V2LS-J3-3.C4 LC
(R19E/K75E/K83E/R105N) (including modified J3).
EVQLVESGGGLVQAGGFLeLSCELRGSIFNQYAMAWFRQAPGKEREFVAGMGAVPHYGEFVKGRFTISRDNA
eSTVYLQMSSLePEDTAIYFCARSKSTYISYNSNGYDYWGnGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 22 is the amino acid sequence of the CAP256V2LS-J3-3.C7 LC
(K75E/K83E/R105Q) (including modified J3).
EVQLVESGGGLVQAGGFLRLSCELRGSIFNQYAMAWFRQAPGKEREFVAGMGAVPHYGEFVKGRFTISRDNA
ESTVYLQMSSLePEDTAIYFCARSKSTYISYNSNGYDYWGqGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 23 is the amino acid sequence of the CAP256V2LS-J3-3.C8 LC (R19E/K75E/K83E)
(including modified J3).
EVQLVESGGGLVQAGGFLELSCELRGSIFNQYAMAWFRQAPGKEREFVAGMGAVPHYGEFVKGRFTISRDNA
eSTVYLQMSSLePEDTAIYFCARSKSTYISYNSNGYDYWGRGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 24 is the amino acid sequence of the CAP256V2LS-J3-3.C5 LC
(R19E/K62E/K75E/K83E/R105Q) (including modified J3).
EVQLVESGGGLVQAGGFLeLSCELRGSIFNQYAMAWFRQAPGKEREFVAGMGAVPHYGEFVeGRFTISRDNA
eSTVYLQMSSLePEDTAIYFCARSKSTYISYNSNGYDYWGqGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 25 is the amino acid sequence of the CAP256V2LS-J3-3.C6 LC
(R19E/K62E/K75E/K83E/R105N) (including modified J3).
EVQLVESGGGLVQAGGFLeLSCELRGSIFNQYAMAWFRQAPGKEREFVAGMGAVPHYGEFVeGRFTISRDNA
eSTVYLQMSSLePEDTAIYFCARSKSTYISYNSNGYDYWGnGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
SEQ ID NO: 26 is the amino acid sequence of J3 VHH with R19E/K83E/R105Q mutations.
EVQLVESGGGLVQAGGFLeLSCELRGSIFNQYAMAWFRQAPGKEREFVAGMGAVPHYGEFVKGRFTISRDNA
KSTVYLQMSSLePEDTAIYFCARSKSTYISYNSNGYDYWGqGTQVTVSS
SEQ ID NOs: 27-31 are the amino acid sequences of the CAP256V2LS-J3-1, CAP256V2LS-J3-2,
CAP256V2LS-J3-4, CAP256V2LS-J3-5, and CAP256V2LS-J3-6 light chain (including J3 and
CAP256V2LS LC), respectfully.
SEQ ID NOs: 32-49 are the amino acid sequences of additional bispecific antibody sequences
including CAP256V2LS LC fused to 10E8-, VRC01-, or PGDM-based scFv or VHH domains.
SEQ ID NOs: 50-54 are peptide linker sequences.
SEQ ID NO: 55 is the amino acid sequence of the CAP256V2LS light chain.
QSVLTQPPSVSAAPGQKVTISCSGNTSNIGNNFVSWYQQRPGRAPQLLIYETDKRPSGIPDRFSASKSGTSG
TLAITGLQTGDEADYYCATWAASLSSARVFGTGTQVIVLGQPKVNPTVTLFPPSSEELQANKATLVCLISDF
YPGAVTVAWKADSSPVKAGVETTTPSKQSNNKYAASSYLSLTPEQWKSHRSYSCQVTHEGSTVEKTVAPTEC
S

### DETAILED DESCRIPTION

Prior studies identified the VRC26.25 antibody, which specifically binds to the V1-V2 region of HIV-1 Env, as well as the J3 V_{H}H, which specifically binds to the CD4 binding site of HIV-1 Env, as potent HIV-1 neutralizing antibodies with suboptimal breadth or potency. As described herein, a large-scale screen of VRC26.25 mutations unexpectedly found that the combination of VRC26.25 and J3 V_{H}H into a single bispecific antibody molecule leads to synergistic and beneficial result for HIV-1 neutralization. Combined into a single bispecific antibody, these two binding domains potently neutralize HIV-1 across an astonishingly broad scope of strains. Further studies described herein provide additional modifications to the bispecific antibody to improve half-life while maintaining HIV-1 neutralization potency and breadth. It is believed that one such bispecific antibody—CAP256.J3LS—is one of the most potent and broad HIV-1 neutralizing antibodies identified thus far.

### I. Summary of Terms

Unless otherwise noted, technical terms are used according to conventional usage. Definitions of many common terms in molecular biology may be found in Krebs et al. (eds.), Lewin's genes XII, published by Jones & Bartlett Learning, 2017. As used herein, the singular forms “a,” “an,” and “the,” refer to both the singular as well as plural, unless the context clearly indicates otherwise. For example, the term “an antigen” includes singular or plural antigens and can be considered equivalent to the phrase “at least one antigen.” As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described herein. In case of conflict, the present specification, including explanations of terms, will control. In addition, the materials, methods, and examples are illustrative only and not intended to be limiting. To facilitate review of the various implementations, the following explanations of terms are provided:

About: Unless context indicated otherwise, “about” refers to plus or minus 5% of a reference value. For example, “about” 100 refers to 95 to 105.

Administration: The introduction of an agent, such as a disclosed antibody, into a subject by a chosen route. Administration can be local or systemic. For example, if the chosen route is intravascular, the agent (such as antibody) is administered by introducing the composition into a blood vessel of the subject. Exemplary routes of administration include, but are not limited to, oral, injection (such as subcutaneous, intramuscular, intradermal, intraperitoneal, and intravenous), sublingual, rectal, transdermal (for example, topical), intranasal, vaginal, and inhalation routes.

Amino acid substitution: The replacement of one amino acid in a polypeptide with a different amino acid.

Antibody and Antigen Binding Fragment: An immunoglobulin, antigen-binding fragment, or derivative thereof, that specifically binds and recognizes an analyte (antigen) such as HIV-1 Env. The term “antibody” is used herein in the broadest sense and encompasses various antibody structures, including but not limited to monoclonal antibodies, polyclonal antibodies, multispecific antibodies (e.g., bispecific antibodies), and antigen binding fragments, so long as they exhibit the desired antigen-binding activity.

Non-limiting examples of antibodies include, for example, intact immunoglobulins and variants and fragments thereof that retain binding affinity for the antigen. Examples of antigen binding fragments include but are not limited to Fv, Fab, Fab′, Fab′-SH, F(ab′)₂; diabodies; linear antibodies; single-chain antibody molecules (e.g. scFv); and multispecific antibodies formed from antibody fragments. Antibody fragments include antigen binding fragments either produced by the modification of whole antibodies or those synthesized de novo using recombinant DNA methodologies (see, e.g., Kontermann and Dubel (Eds.), Antibody Engineering, Vols. 1-2, 2^{nd} ed., Springer-Verlag, 2010).

Antibodies also include genetically engineered forms such as chimeric antibodies (such as humanized murine antibodies) and heteroconjugate antibodies (such as bispecific antibodies).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Typically, a naturally occurring immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (k) and kappa (x). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain). In combination, the heavy and the light chain variable regions specifically bind the antigen.

References to “V_{H}” or “V_{H}” refer to the variable region of an antibody heavy chain, including that of an antigen binding fragment, such as Fv, scFv, dsFv or Fab. References to “V_{L}” or “VL” refer to the variable domain of an antibody light chain, including that of an Fv, scFv, dsFv or Fab.

The V_{H} and V_{L} contain a “framework” region interrupted by three hypervariable regions, also called “complementarity-determining regions” or “CDRs” (see, e.g., Kabat et al., Sequences of Proteins of Immunological Interest, 5^{th} ed., NIH Publication No. 91-3242, Public Health Service, National Institutes of Health, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat et al. (Sequences of Proteins of Immunological Interest, ₅ h ed., NIH Publication No. 91-3242, Public Health Service, National Institutes of Health, U.S. Department of Health and Human Services, 1991; “Kabat” numbering scheme), Al-Lazikani et al., (“Standard conformations for the canonical structures of immunoglobulins,” J. Mol. Bio., 273(4):927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27(1):55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a V_{H} CDR3 is the CDR3 from the V_{H} of the antibody in which it is found, whereas a V_{L}CDR1 is the CDR1 from the V_{L} of the antibody in which it is found. Light chain CDRs are sometimes referred to as LCDR1, LCDR2, and LCDR3. Heavy chain CDRs are sometimes referred to as HCDR1, HCDR2, and HCDR3.

In some implementations, a disclosed antibody includes a heterologous constant domain. For example, the antibody includes a constant domain that is different from a native constant domain, such as a constant domain including one or more modifications (such as the “LS” mutation) to increase half-life.

A “monoclonal antibody” is an antibody obtained from a population of substantially homogeneous antibodies, that is, the individual antibodies comprising the population are identical and/or bind the same epitope, except for possible variant antibodies, for example, containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein. In some examples monoclonal antibodies are isolated from a subject. Monoclonal antibodies can have conservative amino acid substitutions which have substantially no effect on antigen binding or other immunoglobulin functions. (See, for example, Greenfield (Ed.), Antibodies: A Laboratory Manual, 2^{nd} ed. New York: Cold Spring Harbor Laboratory Press, 2014.)

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one implementation, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some implementations, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1^{st} Ed. New York: Cold Spring Harbor Laboratory Press, 2004. Print.; Lonberg, Nat. Biotech., 23: 1117-1125, 2005i; Lonenberg, Curr. Opin. Immunol., 20:450-459, 2008).

Biological sample: A sample obtained from a subject. Biological samples include all clinical samples useful for detection of disease or infection (for example, HIV-1 infection) in subjects, including, but not limited to, cells, tissues, and bodily fluids, such as blood, derivatives and fractions of blood (such as serum), cerebrospinal fluid; as well as biopsied or surgically removed tissue, for example tissues that are unfixed, frozen, or fixed in formalin or paraffin. In a particular example, a biological sample is obtained from a subject having or suspected of having an HIV-1 infection.

Bispecific antibody that neutralizes HIV-1: A bispecific antibody that specifically binds to HIV-1 Env in such a way as to inhibit a biological function associated with HIV-1 Env (such as binding to its target receptor). In several implementations, a bispecific antibody that neutralizes HIV-1 reduces the infectious titer of HIV-1.

Broadly neutralizing bispecific antibodies to HIV-1 are distinct from other antibodies to HIV-1 in that they neutralize a high percentage of the many types of HIV-1 in circulation. In some implementations, broadly neutralizing antibodies to HIV-1 are distinct from other antibodies to HIV-1 in that they neutralize a high percentage (such as at least 90%, at least 95%, at least 96%, at least 97%, at least 98%, at least 99%) of the many types of HIV-1 in circulation.

CD4: Cluster of differentiation factor 4 polypeptide; a T-cell surface protein that mediates interaction with the MHC class II molecule. CD4 also serves as the primary receptor site for HIV-1 on T-cells during HIV-1 infection. CD4 is known to bind to gp120 from HIV-1. The known sequence of the CD4 precursor has a hydrophobic signal peptide, an extracellular region of approximately 370 amino acids, a highly hydrophobic stretch with significant identity to the membrane-spanning domain of the class II MHC beta chain, and a highly charged intracellular sequence of 40 resides (Maddon, Cell 42:93, 1985).

Conditions sufficient to form an immune complex: Conditions which allow an antibody to bind to its cognate epitope to a detectably greater degree than, and/or to the substantial exclusion of, binding to substantially all other epitopes. Conditions sufficient to form an immune complex are dependent upon the format of the binding reaction and typically are those utilized in immunoassay protocols or those conditions encountered in vivo. See Greenfield (Ed.), Antibodies: A Laboratory Manual, 2^{nd} ed. New York: Cold Spring Harbor Laboratory Press, 2014, for a description of immunoassay formats and conditions. The conditions employed in the methods are “physiological conditions” which include reference to conditions (e.g., temperature, osmolarity, pH) that are typical inside a living mammal or a mammalian cell. While it is recognized that some organs are subject to extreme conditions, the intra-organismal and intracellular environment normally lies around pH 7 (e.g., from pH 6.0 to pH 8.0, more typically pH 6.5 to 7.5), contains water as the predominant solvent, and exists at a temperature above 0° C. and below 50° C. Osmolarity is within the range that is supportive of cell viability and proliferation.

The formation of an immune complex can be detected through conventional methods, for instance immunohistochemistry (IHC), immunoprecipitation (IP), flow cytometry, immunofluorescence microscopy, ELISA, immunoblotting (for example, Western blot), magnetic resonance imaging (MRI), computed tomography (CT) scans, radiography, and affinity chromatography.

Conjugate: A complex of two molecules linked together, for example, linked together by a covalent bond. In one implementation, an antibody is linked to an effector molecule; for example, an antibody that specifically binds to HIV-1 Env covalently linked to an effector molecule. The linkage can be by chemical or recombinant means. In one implementation, the linkage is chemical, wherein a reaction between the antibody moiety and the effector molecule has produced a covalent bond formed between the two molecules to form one molecule. A peptide linker (short peptide sequence) can optionally be included between the antibody and the effector molecule. Because conjugates can be prepared from two molecules with separate functionalities, such as an antibody and an effector molecule, they are also sometimes referred to as “chimeric molecules.”

Conservative variants: “Conservative” amino acid substitutions are those substitutions that do not substantially affect or decrease a function of a protein, such as the ability of the protein to interact with a target protein. For example, a HIV-1 Env-specific antibody can include up to 1, 2, 3, 4, 5, 6, 7, 8, 9, or up to 10 conservative substitutions compared to a reference antibody sequence and retain specific binding activity for HIV-1 Env binding, and/or HIV-1 neutralization activity. The term conservative variation also includes the use of a substituted amino acid in place of an unsubstituted parent amino acid.

Individual substitutions, deletions or additions which alter, add or delete a single amino acid or a small percentage of amino acids (for instance less than 5%, in some implementations less than 1%) in an encoded sequence are conservative variations where the alterations result in the substitution of an amino acid with a chemically similar amino acid.

The following six groups are examples of amino acids that are considered to be conservative substitutions for one another:
1) Alanine (A), Serine (S), Threonine (T);
2) Aspartic acid (D), Glutamic acid (E);
3) Asparagine (N), Glutamine (Q);
4) Arginine (R), Lysine (K);
5) Isoleucine (I), Leucine (L), Methionine (M), Valine (V); and
6) Phenylalanine (F), Tyrosine (Y), Tryptophan (W).

Contacting: Placement in direct physical association; includes both in solid and liquid form, which can take place either in vivo or in vitro. Contacting includes contact between one molecule and another molecule, for example the amino acid on the surface of one polypeptide, such as an antigen, that contacts another polypeptide, such as an antibody. Contacting can also include contacting a cell for example by placing an antibody in direct physical association with a cell.

Control: A reference standard. In some implementations, the control is a negative control, such as sample obtained from a healthy patient not infected with HIV-1. In other implementations, the control is a positive control, such as a tissue sample obtained from a patient diagnosed with HIV-1 infection. In still other implementations, the control is a historical control or standard reference value or range of values (such as a previously tested control sample, such as a group of HIV-1 patients with known prognosis or outcome, or group of samples that represent baseline or normal values).

A difference between a test sample and a control can be an increase or conversely a decrease. The difference can be a qualitative difference or a quantitative difference, for example a statistically significant difference. In some examples, a difference is an increase or decrease, relative to a control, of at least about 5%, such as at least about 10%, at least about 20%, at least about 30%, at least about 40%, at least about 50%, at least about 60%, at least about 70%, at least about 80%, at least about 90%, at least about 100%, at least about 150%, at least about 200%, at least about 250%, at least about 300%, at least about 350%, at least about 400%, or at least about 500%.

Degenerate variant: In the context of the present disclosure, a “degenerate variant” refers to a polynucleotide encoding a polypeptide (such as an antibody heavy or light chain) that includes a sequence that is degenerate as a result of the genetic code. There are 20 natural amino acids, most of which are specified by more than one codon. Therefore, all degenerate nucleotide sequences encoding a peptide are included as long as the amino acid sequence of the peptide encoded by the nucleotide sequence is unchanged.

Detectable marker: A detectable molecule (also known as a label) that is conjugated directly or indirectly to a second molecule, such as an antibody, to facilitate detection of the second molecule. For example, the detectable marker can be capable of detection by ELISA, spectrophotometry, flow cytometry, microscopy or diagnostic imaging techniques (such as CT scans, MRIs, ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). Methods for using detectable markers and guidance in the choice of detectable markers appropriate for various purposes are discussed for example in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Detecting: To identify the existence, presence, or fact of something.

Effective amount: A quantity of a specific substance sufficient to achieve a desired effect in a subject to whom the substance is administered. For instance, this can be the amount necessary to prevent, treat (including prophylaxis), reduce and/or ameliorate the symptoms or underlying causes of a disorder or disease, such as HIV-1 infection.

In some implementations, an effective amount of a disclosed bispecific antibody is sufficient to reduce or eliminate a symptom of HIV-1 infection, such as AIDS. For instance, this can be the amount necessary to inhibit or prevent HIV-1 replication or to measurably alter outward symptoms of the HIV-1 infection. Ideally, the effective amount provides a therapeutic effect without causing a substantial cytotoxic effect in the subject.

In some implementations, administration of an effective amount of a disclosed bispecific antibody that binds to HIV-1 Env can reduce or inhibit an HIV-1 infection (for example, as measured by infection of cells, or by number or percentage of subjects infected by HIV-1, or by an increase in the survival time of infected subjects) by a desired amount, for example by at least 10%, at least 20%, at least 50%, at least 60%, at least 70%, at least 80%, at least 90%, at least 95%, at least 98%, or even at least 100% (elimination or prevention of detectable HIV-1 infection), as compared to a suitable control.

An effective amount of an disclosed bispecific antibody that specifically binds gp120 that is administered to a subject will vary depending upon a number of factors associated with that subject, for example the overall health and/or weight of the subject. An effective amount can be determined by varying the dosage and measuring the resulting therapeutic response, such as, for example, a reduction in viral titer. Therapeutically effective amounts also can be determined through various in vitro, in vivo or in situ immunoassays.

An effective amount encompasses a fractional dose that contributes in combination with previous or subsequent administrations to attaining a therapeutic response. For example, an effective amount of an agent can be administered in a single dose, or in several doses, for example daily, during a course of treatment lasting several days or weeks. However, the effective amount can depend on the subject being treated, the severity and type of the condition being treated, and the manner of administration. A unit dosage form of the agent can be packaged in a therapeutic amount, or in multiples of the therapeutic amount, for example, in a vial (e.g., with a pierceable lid) or syringe having sterile components.

Effector molecule: A molecule intended to have or produce a desired effect; for example, a desired effect on a cell to which the effector molecule is targeted, or a detectable marker. Effector molecules can include, for example, polypeptides and small molecules. Some effector molecules may have or produce more than one desired effect.

Epitope: An antigenic determinant. These are particular chemical groups or peptide sequences on a molecule that are antigenic, i.e. that elicit a specific immune response. An antibody specifically binds a particular antigenic epitope on a polypeptide. In some examples a disclosed bispecific antibody specifically binds to two different epitopes on HIV-1 Env.

Expression: Transcription or translation of a nucleic acid sequence. For example, an encoding nucleic acid sequence (such as a gene) can be expressed when its DNA is transcribed into RNA or an RNA fragment, which in some examples is processed to become mRNA. An encoding nucleic acid sequence (such as a gene) may also be expressed when its mRNA is translated into an amino acid sequence, such as a protein or a protein fragment. In a particular example, a heterologous gene is expressed when it is transcribed into an RNA. In another example, a heterologous gene is expressed when its RNA is translated into an amino acid sequence. Regulation of expression can include controls on transcription, translation, RNA transport and processing, degradation of intermediary molecules such as mRNA, or through activation, inactivation, compartmentalization or degradation of specific protein molecules after they are produced.

Expression Control Sequences: Nucleic acid sequences that regulate the expression of a heterologous nucleic acid sequence to which it is operatively linked. Expression control sequences are operatively linked to a nucleic acid sequence when the expression control sequences control and regulate the transcription and, as appropriate, translation of the nucleic acid sequence. Thus, expression control sequences can include appropriate promoters, enhancers, transcriptional terminators, a start codon (ATG) in front of a protein-encoding gene, splice signals for introns, maintenance of the correct reading frame of that gene to permit proper translation of mRNA, and stop codons. The term “control sequences” is intended to include, at a minimum, components whose presence can influence expression, and can also include additional components whose presence is advantageous, for example, leader sequences and fusion partner sequences. Expression control sequences can include a promoter.

Expression vector: A vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Non-limiting examples of expression vectors include cosmids, plasmids (e.g., naked or contained in liposomes) and viruses (e.g., lentiviruses, retroviruses, adenoviruses, and adeno-associated viruses) that incorporate the recombinant polynucleotide.

A polynucleotide can be inserted into an expression vector that contains a promoter sequence which facilitates the efficient transcription of the inserted genetic sequence of the host. The expression vector typically contains an origin of replication, a promoter, as well as specific nucleic acid sequences that allow phenotypic selection of the transformed cells.

Fc region: The constant region of an antibody excluding the first heavy chain constant domain. Fc region generally refers to the last two heavy chain constant domains of IgA, IgD, and IgG, and the last three heavy chain constant domains of IgE and IgM. An Fc region may also include part or all of the flexible hinge N-terminal to these domains. For IgA and IgM, an Fc region may or may not include the tailpiece, and may or may not be bound by the J chain. For IgG, the Fc region is typically understood to include immunoglobulin domains Cγ2 and Cγ3 and optionally the lower part of the hinge between Cγ1 and Cγ2. Although the boundaries of the Fc region may vary, the human IgG heavy chain Fc region is usually defined to include residues following C226 or P230 to the Fc carboxyl-terminus, wherein the numbering is according to EU numbering. For IgA, the Fc region includes immunoglobulin domains Cα2 and Cα3 and optionally the lower part of the hinge between Cα1 and Cα2.

Heterologous: Originating from a different genetic source. A nucleic acid molecule that is heterologous to a cell originated from a genetic source other than the cell in which it is expressed. In one specific, non-limiting example, a heterologous nucleic acid molecule encoding a protein, such as an scFv, is expressed in a cell, such as a mammalian cell. Methods for introducing a heterologous nucleic acid molecule in a cell or organism are well known in the art, for example transformation with a nucleic acid, including electroporation, lipofection, particle gun acceleration, and homologous recombination.

HIV-1 Envelope protein (Env): The HIV-1 envelope protein is initially synthesized as a precursor protein of 845-870 amino acids in size, designated gp160. Individual gp160 polypeptides form a homotrimer and undergo glycosylation within the Golgi apparatus as well as processing to remove the signal peptide, and cleavage by a cellular protease between approximately positions 511/512 to generate separate gp120 and gp41 polypeptide chains, which remain associated as gp120/gp41 protomers within the homotrimer. The ectodomain (that is, the extracellular portion) of the HIV-1 Env trimer undergoes several structural rearrangements from a prefusion mature (cleaved) closed conformation that evades antibody recognition, through intermediate conformations that bind to receptors CD4 and co-receptor (either CCR5 or CXCR4), to a postfusion conformation.

HIV-1 gp120: A polypeptide that is part of the HIV-1 Env protein. Mature gp120 includes approximately HIV-1 Env residues 31-511, contains most of the external, surface-exposed, domains of the HIV-1 Env trimer, and it is gp120 which binds both to cellular CD4 receptors and to cellular chemokine receptors (such as CCR5). A mature gp120 polypeptide is an extracellular polypeptide that interacts with the gp41 ectodomain to form an HIV-1 Env protomer that trimerizes to form the HIV-1 Env trimer.

HIV-1 gp41: A polypeptide that is part of the HIV-1 Env protein. Mature gp41 includes approximately HIV-1 Env residues 512-860, and includes cytosolic-, transmembrane-, and ecto-domains. The gp41 ectodomain (including approximately HIV-1 Env residues 512-644) can interact with gp120 to form an HIV-1 Env protomer that trimerizes to form the HIV-1 Env trimer.

Human Immunodeficiency Virus type 1 (HIV-1): A retrovirus that causes immunosuppression in humans (HIV-1 disease), and leads to a disease complex known as the acquired immunodeficiency syndrome (AIDS). “HIV-1 disease” refers to a well-recognized constellation of signs and symptoms (including the development of opportunistic infections) in persons who are infected by an HIV-1 virus, as determined by antibody or western blot studies. Laboratory findings associated with this disease include a progressive decline in T cells. Related viruses that are used as animal models include simian immunodeficiency virus (SIV) and feline immunodeficiency virus (FIV). Treatment of HIV-1 with HAART has been effective in reducing the viral burden and ameliorating the effects of HIV-1 infection in infected individuals.

Host cell: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

IgA: A polypeptide belonging to the class of antibodies that are substantially encoded by a recognized immunoglobulin alpha gene. In humans, this class or isotype comprises IgA₁ and IgA₂. IgA antibodies can exist as monomers, polymers (referred to as pIgA) of predominantly dimeric form, and secretory IgA. The constant chain of wild-type IgA contains an 18-amino-acid extension at its C-terminus called the tail piece (tp). Polymeric IgA is secreted by plasma cells with a 15-kDa peptide called the J chain linking two monomers of IgA through the conserved cysteine residue in the tail piece.

IgG: A polypeptide belonging to the class or isotype of antibodies that are substantially encoded by a recognized immunoglobulin gamma gene. In humans, this class comprises IgG1, IgG2, IgG3, and IgG4.

Immune complex: The binding of antibody to a soluble antigen forms an immune complex. The formation of an immune complex can be detected through conventional methods, for instance immunohistochemistry, immunoprecipitation, flow cytometry, immunofluorescence microscopy, ELISA, immunoblotting (for example, Western blot), magnetic resonance imaging, CT scans, radiography, and affinity chromatography.

Inhibiting a disease or condition: Reducing the full development of a disease or condition in a subject, for example, reducing the development of AIDS in a subject infected with HIV-1 or reducing symptoms associated with the HIV-1 infection. This includes neutralizing, antagonizing, prohibiting, preventing, restraining, slowing, disrupting, stopping, or reversing progression or severity of the disease or condition.

Inhibiting a disease or condition includes a prophylactic intervention administered before the disease or condition has begun to develop (for example a treatment initiated in a subject at risk of an HIV-1 infection, but not infected by HIV-1) that reduces subsequent development of the disease or condition and also to amelioration of one or more signs or symptoms of the disease or condition following development. Additionally, inhibiting a disease or condition includes a therapeutic intervention administered after a disease or condition has begun to develop (for example, a treatment administered following diagnosis of a subject with HIV-1 infection) that ameliorates one or more signs or symptoms of the disease or condition in the subject. The term “ameliorating,” with reference to inhibiting a disease or condition refers to any observable beneficial effect of the intervention intended to inhibit the disease or condition. The beneficial effect can be evidenced, for example, by a delayed onset of clinical symptoms of the disease or condition in a susceptible subject, a reduction in severity of some or all clinical symptoms of the disease or condition, a slower progression of the disease or condition, an improvement in the overall health or well-being of the subject, a reduction in infection, or by other parameters that are specific to the particular disease or condition.

Isolated: A biological component (such as a nucleic acid, peptide, protein or protein complex, for example an antibody) that has been substantially separated, produced apart from, or purified away from other biological components in the cell of the organism in which the component naturally occurs, that is, other chromosomal and extra-chromosomal DNA and RNA, and proteins. Thus, isolated nucleic acids, peptides and proteins include nucleic acids and proteins purified by standard purification methods. The term also embraces nucleic acids, peptides and proteins prepared by recombinant expression in a host cell, as well as, chemically synthesized nucleic acids. An isolated nucleic acid, peptide or protein, for example an antibody, can be at least 50%, at least 60%, at least 70%, at least 80%, at least 90%, at least 95%, at least 96%, at least 97%, at least 98%, or at least 99% pure.

Kabat position: A position of a residue in an amino acid sequence that follows the numbering convention delineated by Kabat et al. (Sequences of Proteins of Immunological Interest, 5^{th} Edition, Department of Health and Human Services, Public Health Service, National Institutes of Health, Bethesda, NIH Publication No. 91-3242, 1991).

Linker: A bi-functional molecule that can be used to link two molecules into one contiguous molecule, for example, to link a detectable marker to an antibody. Non-limiting examples of peptide linkers include glycine-serine linkers.

The terms “conjugating,” “joining,” “bonding,” or “linking” can refer to making two molecules into one contiguous molecule; for example, linking two polypeptides into one contiguous polypeptide, or covalently attaching an effector molecule or detectable marker radionuclide or other molecule to a polypeptide, such as an scFv. The linkage can be either by chemical or recombinant means. “Chemical means” refers to a reaction between the antibody moiety and the effector molecule such that there is a covalent bond formed between the two molecules to form one molecule.

Nucleic acid (molecule or sequence): A deoxyribonucleotide or ribonucleotide polymer or combination thereof including without limitation, cDNA, mRNA, genomic DNA, and synthetic (such as chemically synthesized) DNA or RNA. The nucleic acid can be double stranded (ds) or single stranded (ss). Where single stranded, the nucleic acid can be the sense strand or the antisense strand. Nucleic acids can include natural nucleotides (such as A, T/U, C, and G), and can include analogs of natural nucleotides, such as labeled nucleotides.

“cDNA” refers to a DNA that is complementary or identical to an mRNA, in either single stranded or double stranded form.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA produced by that gene produces the protein in a cell or other biological system. Both the coding strand, the nucleotide sequence of which is identical to the mRNA sequence and is usually provided in sequence listings, and non-coding strand, used as the template for transcription, of a gene or cDNA can be referred to as encoding the protein or other product of that gene or cDNA. Unless otherwise specified, a “nucleotide sequence encoding an amino acid sequence” includes all nucleotide sequences that are degenerate versions of each other and that encode the same amino acid sequence. Nucleotide sequences that encode proteins and RNA may include introns.

Operably linked: A first nucleic acid sequence is operably linked with a second nucleic acid sequence when the first nucleic acid sequence is placed in a functional relationship with the second nucleic acid sequence. For instance, a promoter, such as the CMV promoter, is operably linked to a coding sequence if the promoter affects the transcription or expression of the coding sequence. Generally, operably linked DNA sequences are contiguous and, where necessary to join two protein-coding regions, in the same reading frame.

Pharmaceutically acceptable carriers: The pharmaceutically acceptable carriers of use are conventional. Remington: The Science and Practice of Pharmacy, 22^{nd} ed., London, UK: Pharmaceutical Press, 2013, describes compositions and formulations suitable for pharmaceutical delivery of the disclosed agents.

In general, the nature of the carrier will depend on the particular mode of administration being employed. For instance, parenteral formulations usually include injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically neutral carriers, pharmaceutical compositions to be administered can contain minor amounts of non-toxic auxiliary substances, such as wetting or emulsifying agents, added preservatives (such as non-natural preservatives), and pH buffering agents and the like, for example sodium acetate or sorbitan monolaurate. In particular examples, the pharmaceutically acceptable carrier is sterile and suitable for parenteral administration to a subject for example, by injection. In some implementations, the active agent and pharmaceutically acceptable carrier are provided in a unit dosage form such as a pill or in a selected quantity in a vial. Unit dosage forms can include one dosage or multiple dosages (for example, in a vial from which metered dosages of the agents can selectively be dispensed).

Polypeptide: A polymer in which the monomers are amino acid residues that are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. A polypeptide includes both naturally occurring proteins, as well as those that are recombinantly or synthetically produced. A polypeptide has an amino terminal (N-terminal) end and a carboxy-terminal end. In some implementations, the polypeptide is a disclosed bispecific antibody.

Purified: The term purified does not require absolute purity; rather, it is intended as a relative term. Thus, for example, a purified peptide preparation is one in which the peptide or protein (such as an antibody) is more enriched than the peptide or protein is in its natural environment within a cell. In one implementation, a preparation is purified such that the protein or peptide represents at least 50% of the total peptide or protein content of the preparation.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, for example, by genetic engineering techniques. A recombinant protein is one that has a sequence that is not naturally occurring or has a sequence that is made by an artificial combination of two otherwise separated segments of sequence. In several implementations, a recombinant protein is encoded by a heterologous (for example, recombinant) nucleic acid that has been introduced into a host cell, such as a bacterial or eukaryotic cell. The nucleic acid can be introduced, for example, on an expression vector having signals capable of expressing the protein encoded by the introduced nucleic acid or the nucleic acid can be integrated into the host cell chromosome.

Sequence identity: The identity between two or more nucleic acid sequences, or two or more amino acid sequences, is expressed in terms of the percentage identity between the sequences. Sequence identity can be measured in terms of percentage identity; the higher the percentage, the more identical the sequences. Homologs and variants of a V_{L} or a V_{H} of an antibody that specifically binds a target antigen are typically characterized by possession of at least about 75% sequence identity, for example at least about 80%, 85%, 90%, 91%, 92%, 93%, 94%, 95%, 96%, 97%, 98% or 99% sequence identity counted over the full-length alignment with the amino acid sequence of interest.

Any suitable method may be used to align sequences for comparison. Non-limiting examples of programs and alignment algorithms are described in: Smith and Waterman, Adv. Appl. Math. 2(4):482-489, 1981; Needleman and Wunsch, J. Mol. Biol. 48(3):443-453, 1970; Pearson and Lipman, Proc. Natl. Acad. Sci. U.S.A. 85(8):2444-2448, 1988; Higgins and Sharp, Gene, 73(1):237-244, 1988; Higgins and Sharp, Bioinformatics, 5(2):151-3, 1989; Corpet, Nucleic Acids Res. 16(22):10881-10890, 1988; Huang et al. Bioinformatics, 8(2):155-165, 1992; and Pearson, Methods Mol. Biol. 24:307-331, 1994., Altschul et al., J. Mol. Biol. 215(3):403-410, 1990, presents a detailed consideration of sequence alignment methods and homology calculations. The NCBI Basic Local Alignment Search Tool (BLAST) (Altschul et al., J. Mol. Biol. 215(3):403-410, 1990) is available from several sources, including the National Center for Biological Information and on the Internet, for use in connection with the sequence analysis programs blastp, blastn, blastx, tblastn, and tblastx. Blastn is used to compare nucleic acid sequences, while blastp is used to compare amino acid sequences. Additional information can be found at the NCBI web site.

Generally, once two sequences are aligned, the number of matches is determined by counting the number of positions where an identical nucleotide or amino acid residue is present in both sequences. The percent sequence identity between the two sequences is determined by dividing the number of matches either by the length of the sequence set forth in the identified sequence, or by an articulated length (such as 100 consecutive nucleotides or amino acid residues from a sequence set forth in an identified sequence), followed by multiplying the resulting value by 100.

Specifically bind: When referring to an antibody or antigen binding fragment, refers to a binding reaction which determines the presence of a target protein in the presence of a heterogeneous population of proteins and other biologics. Thus, under designated conditions, an antibody binds preferentially to a particular target protein, peptide or polysaccharide (such as an antigen present on the surface of a pathogen, for example HIV-1 Env and does not bind in a significant amount to other proteins present in the sample or subject. Specific binding can be determined by standard methods. See Harlow & Lane, Antibodies, A Laboratory Manual, 2^{nd} ed., Cold Spring Harbor Publications, New York (2013), for a description of immunoassay formats and conditions that can be used to determine specific immunoreactivity.

With reference to an antibody-antigen complex, specific binding of the antigen and antibody has a K_{D} of less than about 10⁻⁷ Molar, such as less than about 10⁻⁸ Molar, 10⁻⁹, or even less than about 10⁻¹⁰ Molar. K_{D} refers to the dissociation constant for a given interaction, such as a polypeptide ligand interaction or an antibody antigen interaction. For example, for the bimolecular interaction of an antibody or antigen binding fragment and an antigen it is the concentration of the individual components of the bimolecular interaction divided by the concentration of the complex.

Subject: Living multicellular vertebrate organisms, a category that includes human and non-human mammals. In an example, a subject is a human. In a particular example, the subject is a newborn infant. In an additional example, a subject is selected that is in need of inhibiting an HIV-1 infection. For example, the subject is uninfected and at risk of HIV-1 infection.

Transformed: A transformed cell is a cell into which a nucleic acid molecule has been introduced by molecular biology techniques. As used herein, the term transformed and the like (e.g., transformation, transfection, transduction, etc.) encompasses all techniques by which a nucleic acid molecule might be introduced into such a cell, including transduction with viral vectors, transformation with plasmid vectors, and introduction of DNA by electroporation, lipofection, and particle gun acceleration.

Vector: An entity containing a nucleic acid molecule (such as a DNA or RNA molecule) bearing a promoter(s) that is operationally linked to the coding sequence of a protein of interest and can express the coding sequence. Non-limiting examples include a naked or packaged (lipid and/or protein) DNA, a naked or packaged RNA, a subcomponent of a virus or bacterium or other microorganism that may be replication-incompetent, or a virus or bacterium or other microorganism that may be replication-competent. A vector is sometimes referred to as a construct. Recombinant DNA vectors are vectors having recombinant DNA. A vector can include nucleic acid sequences that permit it to replicate in a host cell, such as an origin of replication. A vector can also include one or more selectable marker genes and other genetic elements. Viral vectors are recombinant nucleic acid vectors having at least some nucleic acid sequences derived from one or more viruses. In some implementations, a viral vector comprises a nucleic acid molecule encoding a disclosed bispecific antibody that specifically binds to HIV-1 Env and neutralizes HIV. In some implementations, the viral vector can be an adeno-associated virus (AAV) vector.

Under conditions sufficient for: A phrase that is used to describe any environment that permits a desired activity.

### II. Description of Several Implementations

Isolated bispecific antibodies that that specifically bind to the CD4 binding site and the V1-V2 region of HIV-1 Env trimer are provided. The bispecific antibodies neutralize HIV-1. Also disclosed herein are compositions including the bispecific antibodies and a pharmaceutically acceptable carrier. Nucleic acids encoding the bispecific antibodies and expression vectors (such as adeno-associated virus (AAV) viral vectors) including these nucleic acids are also provided.

The bispecific antibodies, nucleic acid molecules, and compositions can be used for research, diagnostic and therapeutic purposes. For example, the bispecific antibodies can be used to diagnose or treat a subject with an HIV-1 infection, or can be administered prophylactically to prevent HIV-1 infection in a subject.

### A. Novel Bispecific Antibodies

In some implementations, a bispecific antibody is provided, comprising a first binding domain and a second binding domain. The first binding domain comprises an antibody comprising a heavy chain variable region (V_{H}) comprising a heavy chain complementarity determining region (HCDR)1, a HCDR2, and a HCDR3 comprising amino acid sequences set forth as SEQ ID NOs: 2, 3, and 4, respectively, a light chain variable region (V_{L}), and a constant domain, and specifically binds to the V1-V2 region of HIV-1 Env. The second binding domain comprises a V_{H}H comprising a HCDR1, a HCDR2, and a HCDR3 comprising amino acid sequences set forth as SEQ ID NOs: 10, 11, and 12, respectively, and specifically binds to a CD4 binding site of HIV-1 Env. The first and second binding domains can simultaneously bind to a single HIV-1 Env trimer. The C-terminus of the V_{H}H is fused to the N terminus of the V_{L} by a peptide linker to form a single polypeptide chain. The bispecific antibody neutralizes HIV-1.

In some implementations, the V_{H}H of the second binding domain comprises the HCDR1, HCDR2, and HCDR3 set forth as SEQ ID NOs: 10, 11, and 12, respectively, and the remainder of the V_{H}H of the second binding domain is at least 90% (such as at least 95% or at least 99%) identical to SEQ ID NO: 9. In some implementations, the V_{H}H of the second binding domain comprises an amino acid sequence set forth as SEQ ID NO: 9.

In some implementations, the V_{H}H of the second binding domain comprises the HCDR1, HCDR2, and HCDR3 set forth as SEQ ID NOs: 10, 11, and 12, respectively, and the remainder of the V_{H}H of the second binding domain is at least 90% (such as at least 95% or at least 99%) identical to SEQ ID NO: 9, and further comprises one or more amino acid substitutions in the framework regions to replace positively charged amino acids with neutral or negatively charged amino acids. In some implementations, the V_{H}H of the second binding domain comprises one or more of R19E, K75E, K83E, R105N, R105Q substitutions with reference to SEQ ID NO: 9. In some implementations, the V_{H}H comprises R19E, K83E, and R105Q substitutions with reference to SEQ ID NO: 9. In some implementations, the V_{H}H comprises or consists of the amino acid sequence set forth as SEQ ID NO: 1 or SEQ ID NO: 9 or amino acids 1-121 of any one of SEQ ID NOs: 17-23.

The peptide linker joining the V_{H}H of the second binding domain to the light chain of the first binding domain can have any suitable length or composition of amino acids that allows for the bispecific antibody to simultaneously bind to the CD4 binding site (via the V_{H}H of the second binding domain) and the V1-V2 region (via the antibody of the first binding domain) of HIV-1 Env trimer. In some implementations, the peptide linker is from 5-30 amino acids in length, such as from 10⁻³⁰, from 15-30, from 10⁻²⁰, from 20-30, from 12-17, or from 14-16 amino acids in length. In some implementations, the peptide linker is 5, 10, 15, 20, 25, or 30 amino acids in length. In some implementations, the peptide linker is a glycine-serine linker. In some implementations, the peptide linker comprises a human IgG1 hinge sequence or a multiple thereof, such as from 1-3 repeats of the hinge sequence. In some implementations, the peptide linker comprises or consists of an amino acid sequence set forth as any one of the following: GGSGG (SEQ ID NO: 50), GGSGGGGSGG (SEQ ID NO: 51), GGSGGGGSGGGGSGG (SEQ ID NO: 16), DKTHT (SEQ ID NO: 52), DKTHTGDKTHT (SEQ ID NO: 53), or DKTHTGDKTHTGDKTHT (SEQ ID NO: 54). In several implementations, the peptide liner comprises or consists of the amino acid sequence set forth as SEQ ID NO: 16 (GGSGGGGSGGGGSGG).

In some implementations, the V_{L} of the antibody of the first binding domain comprises a light chain complementarity determining region (LCDR)1, a LCDR2, and a LCDR3 comprising amino acid sequences set forth as SEQ ID NOs: 6, 7, and 8, respectively. In some implementations, the V_{L} of the antibody of the first binding domain comprises an amino acid sequence at least 90% identical to SEQ ID NO: 5. In some implementations, the V_{L} of the antibody of the first binding domain comprises a LCDR1, a LCDR2, and a LCDR3 comprising amino acid sequences set forth as SEQ ID NOs: 6, 7, and 8, respectively, and the remaining amino acid sequence of the V_{L} is at least 90% (such as at least 95% or at least 99%) identical to SEQ ID NO: 5. In some implementations, the V_{L} of the antibody of the first binding domain comprises an amino acid sequence set forth as SEQ ID NO: 5.

In some implementations, the fusion protein including the V_{H}H of the second binding domain fused to the V_{L} of the light chain of the antibody of the first binding domain by the peptide linker comprises the amino acid sequence set forth as any one of residues 1-258 of SEQ ID NOs: 15 or 17-23. In some implementations, the fusion protein including the V_{H}H of the second binding domain fused to the light chain of the antibody of the first binding domain by the peptide linker comprises the amino acid sequence set forth as any one of SEQ ID NOs: 15 or 17-23.

In some implementations, the V_{H} of the antibody of the first binding domain comprises the HCDR1, HCDR2, and HCDR3 set forth as SEQ ID NOs: 2, 3, and 4, respectively, and the remainder of the V_{H} is at least 90% (such as at least 95% or at least 99%) identical to SEQ ID NO: 1. In some implementations, the V_{H} of the antibody of the first binding domain comprises an amino acid sequence set forth as SEQ ID NO: 1.

The antibody of the first binding domain can be in any suitable format that allows for fusion of the V_{H}H to the N-terminus f the light chain of the antibody and for simultaneous binding of the V_{H}H to the CD4 binding site of HIV-1 Env and the antibody of the first binding domain to the V1-V2 region of HIV-1 Env. In some implementations, the antibody has an IgG, IgM or IgA format. In several implementations, the antibody of the first binding domain comprises a constant domain comprising a modification that increases binding to the neonatal Fc receptor. In some implementations, the constant domain comprises M428L and N434S mutations (“LS” mutation) according to EU numbering.

In some implementations, the heavy chain of the antibody of the first binding domain comprises the amino acid sequence set forth as SEQ ID NO: 14 and the V_{H}H fused to the light chain of the antibody comprises the amino acid sequence set forth as any one of SEQ ID NOs: 15 or 17-23. In some implementations, the heavy chain of the antibody of the first binding domain comprises the amino acid sequence set forth as SEQ ID NO: 14 and the V_{H}H fused to the light chain of the antibody comprises the amino acid sequence set forth as SEQ ID NO: 17.

In some implementations, the heavy chain of the antibody of the first binding domain comprises the amino acid sequence set forth as SEQ ID NO: 14 and the V_{H}H fused to the light chain of the antibody comprises the amino acid sequence set forth as any one of SEQ ID NOs: 15 or 27-49.

The V_{H}H of the second binding domain and the V_{L} and V_{H} of the first binding domain can include any suitable framework region, such as (but not limited to) a human framework region.

The antibody of the first binding domain can be of any isotype. The antibody can be, for example, an IgM or an IgG antibody, such as IgG1, IgG2, IgG3, or IgG4. The class of an antibody that specifically binds HIV-1 Env can be switched with another. In one aspect, a nucleic acid molecule encoding V_{L} or V_{H} is isolated using methods well-known in the art, such that it does not include any nucleic acid sequences encoding the constant region of the light or heavy chain, respectively. A nucleic acid molecule encoding V_{L} or V_{H} is then operatively linked to a nucleic acid sequence encoding a C_{L} or C_{H} from a different class of immunoglobulin molecule. This can be achieved using a vector or nucleic acid molecule that comprises a C_{L} or C_{H} chain, as known in the art. For example, an antibody that specifically binds HIV-1 Env, that was originally IgG may be class switched to an IgM. Class switching can be used to convert one IgG subclass to another, such as from IgG1 to IgG2, IgG3, or IgG4.

The bispecific antibody can be derivatized or linked to another molecule (such as another peptide or protein). In general, the bispecific antibody is derivatized such that the binding to HIV-1 Env is not affected adversely by the derivatization or labeling. For example, the bispecific antibody can be functionally linked (by chemical coupling, genetic fusion, noncovalent association or otherwise) to one or more other molecular entities, such as another antibody, a detectable marker, an effector molecule, or a protein or peptide that can mediate association of the bispecific antibody with another molecule (such as a streptavidin core region or a polyhistidine tag).

In several implementations, the bispecific antibody specifically binds HIV-1 Env with an affinity (e.g., measured by K_{D}) of no more than 25 nM, such as no more than 20 nM, no more than 15 nM or no more than 10 nM. K_{D} can be measured, for example, by a radiolabeled antigen binding assay (RIA) performed with the Fab version of an antibody of interest and its antigen, or isothermal calorimetry (ITC) using known methods.

In some implementations, the bispecific antibody can be distinguished by neutralization breadth. In some implementations, the bispecific antibody neutralizes at least 95% (such as at least 96%, at least 97%, at least 98% or at least 99%) of the HIV-1 isolates included in a standardized panel of HIV-1 pseudoviruses (such as the panel of 208 diverse HIV-1 pseudoviruses described in Kwon et al., Cell Reports, 22, 1798-1809, 2018) with an IC₅0 of less than 50 μg/ml. The person of ordinary skill in the art is familiar with methods of measuring neutralization breadth and potency, for example such methods include the single-round HIV-1 Env-pseudoviruses infection of TZM-bl cells (see, e.g., Li et al., J Virol 79, 10108-10125, 2005; see also, PCT Pub. No. WO2011/038290).

In some implementations, amino acid sequence variants of the bispecific antibodies provided herein are provided. For example, it may be desirable to improve the binding affinity and/or other biological properties of the bispecific antibody. Amino acid sequence variants of the bispecific antibody may be prepared by introducing appropriate modifications into the nucleotide sequence encoding the antibody V_{H} domain and/or V_{L} domain, or the V_{H}H. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

In some implementations, variants having one or more amino acid substitutions are provided. Sites of interest for substitutional mutagenesis include the CDRs and the framework regions. Amino acid substitutions may be introduced into an antibody of interest and the products screened for a desired activity, e.g., retained/improved antigen binding, decreased immunogenicity, or improved ADCC or CDC.

The variants typically retain amino acid residues necessary for correct folding and stabilizing between the V_{H} and the V_{L} regions, and will retain the charge characteristics of the residues in order to preserve the low pI and low toxicity of the molecules. Amino acid substitutions can be made in the V_{H} and the V_{L} regions, the constant region, or the V_{H}H, to increase yield.

In some implementations, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. In some implementations of the variant V_{H}H, V_{H}, and V_{L} sequences provided above, each CDR either is unaltered, or contains no more than one, two or three amino acid substitutions. In some implementations of the variant V_{H} and V_{L} sequences provided above, only the framework residues are modified so the CDRs are unchanged.

To increase binding affinity of the antibody, V_{H}H, or the V_{L} and V_{H} segments can be randomly mutated in a process analogous to the in vivo somatic mutation process responsible for affinity maturation of antibodies during a natural immune response. Thus in vitro affinity maturation can be accomplished by amplifying V_{H}H, V_{H} and V_{L} regions using PCR primers complementary to selected regions, such as the HCDR3. In this process, the primers have been “spiked” with a random mixture of the four nucleotide bases at certain positions such that the resultant PCR products encode V_{H}, V_{H} and V_{L} segments into which random mutations have been introduced into the CDR3 regions. These randomly mutated V_{H}H, V_{H} and V_{L} segments can be tested to determine the binding affinity for HIV-1 Env.

In some implementations, a bispecific antibody disclosed herein is altered to increase or decrease the extent to which the antibody is glycosylated. Addition or deletion of glycosylation sites may be conveniently accomplished by altering the amino acid sequence such that one or more glycosylation sites is created or removed.

Where the bispecific antibody comprises an Fc region, the carbohydrate attached thereto may be altered. Native antibodies produced by mammalian cells typically comprise a branched, biantennary oligosaccharide that is generally attached by an N-linkage to Asn297 of the CH₂ domain of the Fc region. See, e.g., Wright et al. Trends Biotechnol. 15(1):26-32, 1997. The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some implementations, modifications of the oligosaccharide in an antibody may be made in order to create antibody variants with certain improved properties.

In one implementation, variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e.g. complex, hybrid and high mannose structures) as measured by MALDI-TOF mass spectrometry, as described in WO 2008/077546, for example. Asn297 refers to the asparagine residue located at about position 297 in the Fc region; however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO 2002/031140; Okazaki et al., J. Mol. Biol., 336(5):1239-1249, 2004; Yamane-Ohnuki et al., Biotechnol. Bioeng. 87(5):614-622, 2004. Examples of cell lines capable of producing defucosylated antibodies include Lee 13 CHO cells deficient in protein fucosylation (Ripka et al., Arch. Biochem. Biophys. 249(2):533-545, 1986; US Pat. Appl. No. US 2003/0157108 and WO 2004/056312, especially at Example 11), and knockout cell lines, such as alpha-1,6-fucosyltransferase gene, FUT8, knockout CHO cells (see, e.g., Yamane-Ohnuki et al., Biotechnol. Bioeng., 87(5): 614-622, 2004; Kanda et al., Biotechnol. Bioeng., 94(4):680-688, 2006; and WO2003/085107).

Antibody variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In several implementations, the constant region of the antibody comprises one or more amino acid substitutions to optimize in vivo half-life of the antibody. The serum half-life of IgG Abs is regulated by the neonatal Fc receptor (FcRn). Thus, in several implementations, the antibody comprises an amino acid substitution that increases binding to the FcRn. Non-limiting examples of such substitutions include substitutions at IgG constant regions T250Q and M428L (see, e.g., Hinton et al., J Immunol., 176(1):346-356, 2006); M428L and N434S (numbering according to EU system, the “LS” mutation, see, e.g., Zalevsky, et al., Nature Biotechnol., 28(2):157-159, 2010); N434 Å (see, e.g., Petkova et al., Int. Immunol., 18(12):1759-1769, 2006); T307 Å, E380 Å, and N434 Å (see, e.g., Petkova et al., Int. Immunol., 18(12):1759-1769, 2006); and M252Y, S254T, and T256E (see, e.g., Dall'Acqua et al., J. Biol. Chem., 281(33):23514-23524, 2006). The disclosed antibodies can be linked to or comprise an Fc polypeptide including any of the substitutions listed above, for example, the Fc polypeptide can include the M428L and N434S substitutions.

In some implementations, an bispecific antibody provided herein may be further modified to contain additional nonproteinaceous moieties. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1,3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in an application under defined conditions, etc.

### B. Polynucleotides and Expression

Nucleic acid molecules (for example, cDNA or RNA molecules) encoding the amino acid sequences of the disclosed bispecific antibodies are provided. Nucleic acids encoding these molecules can readily be produced using the amino acid sequences provided herein (such as the CDR sequences and V_{H}, V_{L}, and V_{H}H sequences), sequences available in the art (such as framework or constant region sequences), and the genetic code. In several implementations, nucleic acid molecules can encode the V_{H}, the V_{H}H fused to the V_{L}, or both the V_{H} and the V_{H}H fused to the V_{L} (for example in a bicistronic expression vector) of a disclosed bispecific antibody. In several implementations, the nucleic acid molecules can be expressed in a host cell (such as a mammalian cell) to produce a disclosed antibody.

The genetic code can be used to construct a variety of functionally equivalent nucleic acid sequences, such as nucleic acids which differ in sequence but which encode the same antibody sequence or a conjugate or fusion protein including the V_{H}H fused to the V_{L} and/or V_{H} of the bispecific antibody.

In a non-limiting example, an isolated nucleic acid molecule encodes the V_{H} of a disclosed bispecific antibody. In another non-limiting example, the nucleic acid molecule encodes the V_{H}H fused to the V_{L} of a disclosed bispecific antibody.

Nucleic acid molecules encoding the bispecific antibodies and conjugates that specifically bind HIV-1 Env can be prepared by any suitable method including, for example, cloning of appropriate sequences or by direct chemical synthesis by standard methods. Chemical synthesis produces a single stranded oligonucleotide. This can be converted into double stranded DNA by hybridization with a complementary sequence or by polymerization with a DNA polymerase using the single strand as a template.

Exemplary nucleic acids can be prepared by cloning techniques. Examples of appropriate cloning and sequencing techniques can be found, for example, in Green and Sambrook (Molecular Cloning: A Laboratory Manual, 4^{th} ed., New York: Cold Spring Harbor Laboratory Press, 2012) and Ausubel et al. (Eds.) (Current Protocols in Molecular Biology, New York: John Wiley and Sons, including supplements, 2017).

Nucleic acids can also be prepared by amplification methods. Amplification methods include the polymerase chain reaction (PCR), the ligase chain reaction (LCR), the transcription-based amplification system (TAS), and the self-sustained sequence replication system (3SR).

The nucleic acid molecules can be expressed in a recombinantly engineered cell such as bacteria, plant, yeast, insect and mammalian cells. The bispecific antibodies can be expressed as individual proteins including the V_{H} and/or the V_{H}H fused to the V_{L} (linked to an effector molecule or detectable marker as needed), or can be expressed as a fusion protein. Any suitable method of expressing and purifying antibodies may be used; non-limiting examples are provided in Al-Rubeai (Ed.), Antibody Expression and Production, Dordrecht; New York: Springer, 2011). An immunoadhesin can also be expressed. Thus, in some examples, nucleic acids encoding a V_{H} and the V_{H}H fused to the V_{L}, and immunoadhesin are provided. The nucleic acid sequences can optionally encode a leader sequence.

One or more DNA sequences encoding the antibodies or conjugates can be expressed in vitro by DNA transfer into a suitable host cell. The cell may be prokaryotic or eukaryotic. Numerous expression systems available for expression of proteins including E. coli, other bacterial hosts, yeast, and various higher eukaryotic cells such as the COS, CHO, HeLa and myeloma cell lines, can be used to express the disclosed bispecific antibodies. Methods of stable transfer, meaning that the foreign DNA is continuously maintained in the host, are known in the art. Hybridomas expressing the antibodies of interest are also encompassed by this disclosure.

The expression of nucleic acids encoding the bispecific antibodies described herein can be achieved by operably linking the DNA or cDNA to a promoter (which is either constitutive or inducible), followed by incorporation into an expression cassette. The promoter can be any promoter of interest, including a cytomegalovirus promoter. Optionally, an enhancer, such as a cytomegalovirus enhancer, is included in the construct. The cassettes can be suitable for replication and integration in either prokaryotes or eukaryotes. Typical expression cassettes contain specific sequences useful for regulation of the expression of the DNA encoding the protein. For example, the expression cassettes can include appropriate promoters, enhancers, transcription and translation terminators, initiation sequences, a start codon (i.e., ATG) in front of a protein-encoding gene, splicing signals for introns, sequences for the maintenance of the correct reading frame of that gene to permit proper translation of mRNA, and stop codons. The vector can encode a selectable marker, such as a marker encoding drug resistance (for example, ampicillin or tetracycline resistance).

To obtain high level expression of a cloned gene, it is desirable to construct expression cassettes which contain, for example, a strong promoter to direct transcription, a ribosome binding site for translational initiation (e.g., internal ribosomal binding sequences), and a transcription/translation terminator. For E. coli, this can include a promoter such as the T7, trp, lac, or lamda promoters, a ribosome binding site, and preferably a transcription termination signal. For eukaryotic cells, the control sequences can include a promoter and/or an enhancer derived from, for example, an immunoglobulin gene, HTLV, SV40 or cytomegalovirus, and a polyadenylation sequence, and can further include splice donor and/or acceptor sequences (for example, CMV and/or HTLV splice acceptor and donor sequences). The cassettes can be transferred into the chosen host cell by any suitable method such as transformation or electroporation for E. coli and calcium phosphate treatment, electroporation or lipofection for mammalian cells. Cells transformed by the cassettes can be selected by resistance to antibiotics conferred by genes contained in the cassettes, such as the amp, gpt, neo and hyg genes.

Modifications can be made to a nucleic acid encoding a polypeptide described herein without diminishing its biological activity. Some modifications can be made to facilitate the cloning, expression, or incorporation of the targeting molecule into a fusion protein. Such modifications include, for example, termination codons, sequences to create conveniently located restriction sites, and sequences to add a methionine at the amino terminus to provide an initiation site, or additional amino acids (such as poly His) to aid in purification steps.

Once expressed, the bispecific antibodies and conjugates can be purified according to standard procedures in the art, including ammonium sulfate precipitation, affinity columns, column chromatography, and the like (see, generally, Simpson et al. (Eds.), Basic methods in Protein Purification and Analysis: A Laboratory Manual, New York: Cold Spring Harbor Laboratory Press, 2009). The bispecific antibodies and conjugates need not be 100% pure. Once purified, partially or to homogeneity as desired, if to be used prophylatically, the polypeptides should be substantially free of endotoxin.

Methods for expression of antibodies and conjugates, and/or refolding to an appropriate active form, from mammalian cells, and bacteria such as E. coli have been described and are applicable to the antibodies disclosed herein. See, e.g., Greenfield (Ed.), Antibodies: A Laboratory Manual, 2^{nd} ed. New York: Cold Spring Harbor Laboratory Press, 2014, Simpson et al. (Eds.), Basic methods in Protein Purification and Analysis: A Laboratory Manual, New York: Cold Spring Harbor Laboratory Press, 2009, and Ward et al., Nature 341(6242):544-546, 1989.

### C. Methods and Compositions

### 1. Therapeutic Methods

Methods are disclosed herein for the inhibition of an HIV-1 infection in a subject. The methods include administering to a subject an effective amount (that is, an amount effective to inhibit HIV-1 infection in a subject) of a disclosed bispecific antibody or a nucleic acid encoding the bispecific antibody to a subject with or at risk of the HIV-1 infection. The methods can be used pre-exposure (for example, to prevent HIV-1 infection), in post-exposure prophylaxis, or for treatment of a subject with an HIV-1 infection.

In some examples, the bispecific antibody or nucleic acid molecule can be used to eliminate or reduce the viral reservoir of HIV-1 in a subject.

HIV-1 infection does not need to be completely inhibited for the method to be effective. For example, the method can decrease HIV-1 infection by at least 10%, at least 20%, at least 50%, at least 60%, at least 70%, at least 80%, at least 90%, at least 95%, at least 98%, or even at least 100% (elimination of detectable HIV-1 infected cells), as compared to HIV-1 infection in the absence of the treatment. In some implementations, the method results in a reduction of HIV-1 replication in the subject. HIV-1 replication does not need to be completely eliminated for the method to be effective. For example, the method can reduce HIV-1 replication in the subject by at least 10%, at least 20%, at least 50%, at least 60%, at least 70%, at least 80%, at least 90%, at least 95%, at least 98%, or even at least 100% (elimination of detectable HIV-1 replication), as compared to HIV-1 replication in the absence of the treatment.

In some implementations, administration of an effective amount of a disclosed bispecific antibody or nucleic acid molecule inhibits the establishment of HIV-1 infection and/or subsequent HIV-1 progression in a subject, which can encompass any statistically significant reduction in HIV-1 activity or symptoms of HIV-1 infection in the subject.

In one implementation, administration of a disclosed bispecific antibody or nucleic acid molecule results in a reduction in the establishment of HIV-1 infection and/or reducing subsequent HIV-1 disease progression in a subject. A reduction in the establishment of HIV-1 infection and/or a reduction in subsequent HIV-1 disease progression encompass any statistically significant reduction in HIV-1 activity. In some implementations, methods are disclosed for treating a subject with an HIV-1 infection. These methods include administering to the subject a effective amount of a disclosed bispecific antibody or nucleic acid molecule to preventing or treating the HIV-1 infection.

Studies have shown that the rate of HIV-1 transmission from mother to infant is reduced significantly when zidovudine is administered to HIV-infected women during pregnancy and delivery and to the offspring after birth (Connor et al., 1994 Pediatr Infect Dis J 14: 536-541). The present disclosure provides bispecific antibodies and nucleic acid molecules that are of use in decreasing HIV-transmission from mother to infant. In some examples, an effective amount of a HIV-1 Env-specific antibody or nucleic acid molecule encoding the bispecific antibodies is administered to a pregnant subject in order to prevent transmission of HIV-1, or decrease the risk of transmission of HIV-1, from a mother to an infant. In some examples, an effective amount of the bispecific antibody or nucleic acid encoding the bispecific antibody is administered to mother and/or to the child at childbirth. In other examples, an effective amount of the bispecific antibody or nucleic acid molecule encoding the bispecific antibody is administered to the mother and/or infant prior to breast feeding in order to prevent viral transmission to the infant or decrease the risk of viral transmission to the infant.

For any application, the bispecific antibody or nucleic acid molecule can be combined with anti-retroviral therapy. Antiretroviral drugs are broadly classified by the phase of the retrovirus life-cycle that the drug inhibits. The disclosed bispecific antibodies can be administered in conjunction with nucleoside analog reverse-transcriptase inhibitors (such as zidovudine, didanosine, zalcitabine, stavudine, lamivudine, abacavir, emtricitabine, entecavir, and apricitabine), nucleotide reverse transcriptase inhibitors (such as tenofovir and adefovir), non-nucleoside reverse transcriptase inhibitors (such as efavirenz, nevirapine, delavirdine, etravirine, and rilpivirine), protease inhibitors (such as saquinavir, ritonavir, indinavir, nelfinavir, amprenavir, lopinavir, fosamprenavir, atazanavir, tipranavir, and darunavir), entry or fusion inhibitors (such as maraviroc and enfuvirtide), maturation inhibitors, (such as bevirimat and vivecon), or a broad spectrum inhibitors, such as natural antivirals. In some examples, a disclosed bispecific antibody or nucleic acids encoding such is administered in conjunction with IL-15, or conjugated to IL-15.

Studies have shown that cocktails of HIV-1 neutralizing antibodies that target different epitopes of gp120 can treat macaques chronically infected with SHIV (Shingai et al., Nature, 503, 277-280, 2013; and Barouch et al., Nature, 503, 224-228, 2013). Accordingly, in some examples, a subject is further administered one or more additional antibodies that bind HIV-1 Env (e.g., that bind to gp120 or gp41), and that can neutralize HIV-1. The additional antibodies can be administrated before, during, or after administration of the novel antibodies disclosed herein. In some implementations, the additional antibody can be an antibody that specifically binds to an epitope on HIV-1 Env such as the membrane-proximal external region (e.g., 10E8 antibody), the V1/V2 domain (e.g., PG9 antibody, CAP256-VRC26), or the V3 loop (e.g., 10⁻¹⁰⁷⁴, PGT 121, or PGT128 antibody), or those that bind both gp120 and gp41 subunits (eg. 35022, PGT151, or 8ANC195). Antibodies that specifically bind to these regions and neutralizing HIV-1 infection are known to the person of ordinary skill in the art. Non-limiting examples can be found, for example, in PCT Pub. No. WO 2011/038290, WO/2013/086533, WO/2013/090644, WO/2012/158948.

Antibodies are typically administered by intravenous infusion. Doses of the bispecific antibody vary, but generally range between about 0.5 mg/kg to about 50 mg/kg, such as a dose of about 1 mg/kg, about 5 mg/kg, about 10 mg/kg, about 20 mg/kg, about 30 mg/kg, about 40 mg/kg, or about 50 mg/kg. In some implementations, the dose of the bispecific antibody can be from about 0.5 mg/kg to about 5 mg/kg, such as a dose of about 1 mg/kg, about 2 mg/kg, about 3 mg/kg, about 4 mg/kg or about 5 mg/kg. The bispecific antibody is administered according to a dosing schedule determined by a medical practitioner. In some examples, the bispecific antibody is administered weekly, every two weeks, every three weeks or every four weeks.

In some examples, a subject is administered DNA or RNA encoding a disclosed bispecific antibody to provide in vivo antibody production, for example using the cellular machinery of the subject. Administration of nucleic acid constructs is known in the art and taught, for example, in U.S. Pat. Nos. 5,643,578, 5,593,972 and 5,817,637. U.S. Pat. No. 5,880,103 describes several methods of delivery of nucleic acids encoding proteins to an organism. One approach to administration of nucleic acids is direct administration with plasmid DNA, such as with a mammalian expression plasmid. The nucleotide sequence encoding the disclosed antibody can be placed under the control of a promoter to increase expression. The methods include liposomal delivery of the nucleic acids. Such methods can be applied to the production of a bispecific antibody. In some implementations, a disclosed antibody is expressed in a subject using the pVRC8400 vector (described in Barouch et al., J. Virol., 79(14), 8828-8834, 2005).

In several implementations, a subject (such as a human subject at risk of ebolavirus infection) can be administered an effective amount of an AAV viral vector that includes one or more nucleic acid molecules encoding a disclosed bispecific antibody. The AAV viral vector is designed for expression of the nucleic acid molecules encoding a disclosed bispecific antibody, and administration of the effective amount of the AAV viral vector to the subject leads to expression of an effective amount of the bispecific antibody in the subject. Non-limiting examples of AAV viral vectors that can be used to express a disclosed antibody in a subject include those provided in Johnson et al., Nat. Med., 15(8):901-906, 2009 and Gardner et al., Nature, 519(7541):87-91, 2015.

In one implementation, a nucleic acid encoding a disclosed bispecific antibody is introduced directly into tissue. For example, the nucleic acid can be loaded onto gold microspheres by standard methods and introduced into the skin by a device such as Bio-Rad's HELIOS™ Gene Gun. The nucleic acids can be “naked,” consisting of plasmids under control of a strong promoter. Typically, the DNA is injected into muscle, although it can also be injected directly into other sites. Dosages for injection are usually around 0.5 g/kg to about 50 mg/kg, and typically are about 0.005 mg/kg to about 5 mg/kg (see, e.g., U.S. Pat. No. 5,589,466).

Single or multiple administrations of a composition including a disclosed bispecific specific antibody or nucleic acid molecule can be administered depending on the dosage and frequency as required and tolerated by the patient. The dosage can be administered once, but may be applied periodically until either a desired result is achieved or until side effects warrant discontinuation of therapy. Generally, the dose is sufficient to inhibit ebolavirus infection without producing unacceptable toxicity to the patient.

Data obtained from cell culture assays and animal studies can be used to formulate a range of dosage for use in humans. The dosage normally lies within a range of circulating concentrations that include the ED₅₀, with little or minimal toxicity. The dosage can vary within this range depending upon the dosage form employed and the route of administration utilized. The effective dose can be determined from cell culture assays and animal studies.

The HIV-1 Env-specific bispecific antibody or nucleic acid molecule encoding the bispecific antibody, or a composition including such molecules, can be administered to subjects in various ways, including local and systemic administration, such as, e.g., by injection subcutaneously, intravenously, intra-arterially, intraperitoneally, intramuscularly, intradermally, or intrathecally. In an implementation, the bispecific antibody or nucleic acid molecule, or a composition including such molecules, is administered by a single subcutaneous, intravenous, intra-arterial, intraperitoneal, intramuscular, intradermal or intrathecal injection once a day. The bispecific antibody or nucleic acid molecule, or a composition including such molecules, can also be administered by direct injection at or near the site of disease. A further method of administration is by osmotic pump (e.g., an Alzet pump) or mini-pump (e.g., an Alzet mini-osmotic pump), which allows for controlled, continuous and/or slow-release delivery of the bispecific antibody or nucleic acid molecule or a composition including such molecules, over a pre-determined period. The osmotic pump or mini-pump can be implanted subcutaneously, or near a target site.

### 2. Compositions

Compositions are provided that include the HIV-1 Env-specific bispecific antibody or nucleic acid molecule that are disclosed herein in a carrier. The compositions are useful, for example, for the inhibition or detection of an HIV-1 infection.

The compositions can be prepared in unit dosage forms for administration to a subject. The amount and timing of administration are at the discretion of the administering physician to achieve the desired purposes. The HIV-1 Env-specific bispecific antibody or nucleic acid molecule can be formulated for systemic or local administration. In one example, the HIV-1 Env-specific bispecific antibody or nucleic acid molecule is formulated for parenteral administration, such as intravenous administration.

In some implementations, the bispecific antibody in the composition is at least 70% (such as at least 75%, at least 80%, at least 85%, at least 90%, at least 95%, at least 96%, at least 97%, at least 98% or at least 99%) pure. In some implementations, the composition contains less than 10% (such as less than 5%, less than 4%, less than 3%, less than 2%, less than 1%, less than 0.5%, or even less) of macromolecular contaminants, such as other mammalian (e.g., human) proteins.

The compositions for administration can include a solution of the HIV-1 Env-specific bispecific antibody or nucleic acid molecule dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well-known sterilization techniques. The compositions may contain pharmaceutically acceptable auxiliary substances as required to approximate physiological conditions such as pH adjusting and buffering agents, toxicity adjusting agents and the like, for example, sodium acetate, sodium chloride, potassium chloride, calcium chloride, sodium lactate and the like. The concentration of bispecific antibody in these formulations can vary widely, and will be selected primarily based on fluid volumes, viscosities, body weight and the like in accordance with the particular mode of administration selected and the subject's needs.

A typical composition for intravenous administration includes about 0.01 to about 30 mg/kg of bispecific antibody per subject per day. Actual methods for preparing administrable compositions are known and are described in more detail in such publications as Remington: The Science and Practice of Pharmacy, 22^{nd} ed., London, UK: Pharmaceutical Press, 2013. In some implementations, the composition can be a liquid formulation including one or more bispecific antibodies in a concentration range from about 0.1 mg/ml to about 20 mg/ml, or from about 0.5 mg/ml to about 20 mg/ml, or from about 1 mg/ml to about 20 mg/ml, or from about 0.1 mg/ml to about 10 mg/ml, or from about 0.5 mg/ml to about 10 mg/ml, or from about 1 mg/ml to about 10 mg/ml.

Bispecific antibodies or a nucleic acid molecule can be provided in lyophilized form and rehydrated with sterile water before administration, although they are also provided in sterile solutions of known concentration. The bispecific antibody or nucleic acid molecule solution can then be added to an infusion bag containing 0.9% sodium chloride, USP, and typically administered at a dosage of from 0.5 to 15 mg/kg of body weight. Considerable experience is available in the art in the administration of antibody drugs, which have been marketed in the U.S. since the approval of Rituximab in 1997. Bispecific antibodies or a nucleic acid encoding can be administered by slow infusion, rather than in an intravenous push or bolus. In one example, a higher loading dose is administered, with subsequent, maintenance doses being administered at a lower level. For example, an initial loading dose of 4 mg/kg may be infused over a period of some 90 minutes, followed by weekly maintenance doses for 4-8 weeks of 2 mg/kg infused over a 30-minute period if the previous dose was well tolerated.

Controlled-release parenteral formulations can be made as implants, oily injections, or as particulate systems. For a broad overview of protein delivery systems see, Banga, Therapeutic Peptides and Proteins: Formulation, Processing, and Delivery Systems, Lancaster, PA: Technomic Publishing Company, Inc., 1995. Particulate systems include microspheres, microparticles, microcapsules, nanocapsules, nanospheres, and nanoparticles. Microcapsules contain the active protein agent, such as a cytotoxin or a drug, as a central core. In microspheres, the active protein agent is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, Colloidal Drug Delivery Systems, J. Kreuter (Ed.), New York, NY: Marcel Dekker, Inc., pp. 219-342, 1994; and Tice and Tabibi, Treatise on Controlled Drug Delivery: Fundamentals, Optimization, Applications, A. Kydonieus (Ed.), New York, NY: Marcel Dekker, Inc., pp. 315-339, 1992.

Polymers can be used for ion-controlled release of the bispecific antibody compositions disclosed herein. Various degradable and nondegradable polymeric matrices for use in controlled drug delivery are known in the art (Langer, Acc. Chem. Res. 26(10):537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res., 9(3):425-434, 1992; and Pec et al., J. Parent. Sci. Tech., 44(2):58-65, 1990). Alternatively, hydroxyapatite has been used as a microcarrier for controlled release of proteins (Ijntema et al., Int. J. Pharm.112(3):215-224, 1994). In yet another aspect, liposomes are used for controlled release as well as drug targeting of the lipid-capsulated drug (Betageri et al., Liposome Drug Delivery Systems, Lancaster, PA: Technomic Publishing Co., Inc., 1993). Numerous additional systems for controlled delivery of active protein agent are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

### 3. Methods of Detection and Diagnosis

Methods are also provided for the detection of the presence of HIV-1 Env in vitro or in vivo. In one example, the presence of HIV-1 Env is detected in a biological sample from a subject, and can be used to identify a subject with HIV-1 infection. The sample can be any sample, including, but not limited to, tissue from biopsies, autopsies and pathology specimens. Biological samples also include sections of tissues, for example, frozen sections taken for histological purposes. Biological samples further include body fluids, such as blood, serum, plasma, sputum, spinal fluid or urine. The method of detection can include contacting a cell or sample, with an bispecific antibody that specifically binds to HIV-1 Env or conjugate thereof (e.g. a conjugate including a detectable marker) under conditions sufficient to form an immune complex, and detecting the immune complex (e.g., by detecting a detectable marker conjugated to the bispecific antibody).

In one implementation, the bispecific antibody is directly labeled with a detectable marker. In another implementation, the antibody that binds HIV-1 Env (the primary antibody) is unlabeled and a secondary antibody or other molecule that can bind the primary antibody is utilized for detection. The secondary antibody is chosen that is able to specifically bind the specific species and class of the first antibody. For example, if the first antibody is a human IgG, then the secondary antibody may be an anti-human-IgG. Other molecules that can bind to antibodies include, without limitation, Protein A and Protein G, both of which are available commercially. Suitable labels for the bispecific antibody or secondary antibody are known and described above, and include various enzymes, prosthetic groups, fluorescent materials, luminescent materials, magnetic agents and radioactive materials.

In some implementations, the disclosed bispecific antibodies are used to test vaccines. For example, to test if a vaccine composition including an HIV-1 Env or fragment thereof assumes a conformation including the epitope of a disclosed bispecific antibody. Thus, provided herein is a method for testing a vaccine, wherein the method includes contacting a sample containing the vaccine, such as an HIV-1 Env immunogen, with a disclosed bispecific antibody under conditions sufficient for formation of an immune complex, and detecting the immune complex, to detect the vaccine, such as an HIV-1 Env immunogen including the epitope, in the sample. In one example, the detection of the immune complex in the sample indicates that the vaccine component, such as an HIV-1 Env immunogen, assumes a conformation capable of binding the bispecific antibody.

### III. EXAMPLES

The following examples are provided to illustrate particular features of certain implementations, but the scope of the claims should not be limited to those features exemplified.

### Example 1

### Design and Assessment of a Highly Potent and Broad Bispecific Antibody for HIV-1

This example illustrates the design and assessment of a novel and extraordinarily broad and potent neutralizing antibody for HIV-1.

Antibody CAP256-VRC26.25 targets the second hypervariable region (V2) at the apex of the HIV envelope (Env) trimer with extraordinary neutralization potency, although less than optimal breadth. To improve breadth, we linked the light chain of CAP256V2LS, an optimized version of CAP256-VRC26.25, to the llama nanobody J3, which has broad CD4-binding site-directed neutralization. The J3-linked bispecific antibody exhibited improved breadth and potency over both J3 and CAP256V2LS, indicative of synergistic neutralization. The cryo-EM structure of the bispecific antibody in complex with a prefusion-closed Env trimer revealed simultaneous binding of J3 and CAP256V2LS. We further optimized the pharmacokinetics of the bispecific antibody by reducing the net positive charge of J3. The optimized bispecific antibody, named CAP256.J3LS, had a half-life similar to CAP256V2LS in human FcRn knock-in mice and exhibited suitable auto-reactivity, manufacturability, and biophysical risk. CAP256.J3LS neutralized over 97% of a multiclade 208-strain panel (geometric mean concentration for 80% inhibition (IC₈₀) 0.079 μg/ml) and 100% of a 100-virus clade C panel (geometric mean IC₈₀ of 0.05 μg/ml), suggesting its anti-HIV utility especially in regions where clade C dominates.

### INTRODUCTION

Antibody-mediated prevention (AMP) of HIV-1 infection has been a long-sought goal. AMP clinical studies of VRC01 demonstrate the ability of passively delivered antibodies to prevent HIV-1 infection, but prevention by VRC01 requires a neutralization potency of better than 1 μg/ml 80% of maximal inhibition concentration (IC₈₀). This result has set off a search for other broadly neutralizing antibodies, capable of neutralizing at this potency.

Antibodies against the second hypervariable region (V2), at the trimer apex, are among the most potent broadly neutralizing antibodies identified to date; on a 208-strain panel, V2-apex-directed antibodies such as PG9 and PG16 neutralize at a mean IC₈₀ of 0.34 and 0.113 μg/ml, respectively, PGT145 and its somatic variant PGDM1400 neutralizes at 0.343 and 0.049 μg/ml, respectively, and CAP256-VRC26.25 neutralizes at 0.035 μg/ml. See Chuang et al., Structure. 2019; 27(1):196-206 e6; Walker et al., Science. 2009; 326(5950):285-89; Walker et al., Nature. 2011; 477:466-70; Sok et al., Proc Natl Acad Sci USA. 2014; 111:17624-29; Doria-Rose et al., J Virol. 2016; 90:76-91. However, the neutralization breadths of these antibodies have been less than ideal, ranging from 50% to 80% on the 208-strain panel. Multispecific antibodies could improve both breadth and potency, if the right combination could be identified (along with the right linker) to enable simultaneous engagement of multiple sites of vulnerability, including the potent V2 apex site and other sites more conserved among multiple clade viral strains.

Structures of PG9, PGT145, and CAP256-VRC26.25 in complex with HIV-1 envelope (Env) trimer, or V1V2 scaffolds reveal recognition to occur via an extended 3^{rd} heavy chain complementarity-determining region (CDR H3) (see Gorman et al., Cell Rep. 2020; 31:107488; Lee et al., Immunity. 2017; 46:690-702; McLellan et al. Nature. 2011; 480:336-43), leaving the light chain relatively unencumbered, a potentially ready site for conjugation. Indeed, among the over 2000 non-redundant structures of antibodies bound to antigen, V2-apex-directed antibodies have the longest distance between antibody-framework and antigen, with CAP256-VRC26.25 being the furthest from the antigen (see Lee et al., Nat Commun. 2021;12(1):6470).

Here, we generated bispecific antibodies by genetically fusing single-chain variable regions (scFvs) of human antibodies or single variable domains of heavy-chain-only antibodies (nanobodies) to the light chain N terminus of CAP256V2LS, an optimized version of CAP256-VRC26.25. We assessed binding and neutralization for these bispecific antibodies and structurally characterized the broadest and most potent. We improved pharmacokinetics of these bispecific antibodies by reducing their surface electropositivity and assessed their physical properties, neutralization, manufacturability, and biophysical risk. We identify CAP256.J3LS, a bispecific antibody with a suitable half-life, capable of neutralizing 97% of a 208-virus multiclade panel and 100% of a clade C panel at an IC₈₀ of less than 50 μg/ml, and 77% of the 208-strain panel and 82% of the clade C panel at IC₈₀ of less than 1 μg/ml.

### Results

In an effort to increase the neutralization breadth of CAP256-VRC26.25 while maintaining potency, modified forms of the antibody were designed, produced, and assessed for HIV-1 Env binding and neutralization. Over 120 different modified CAP256-VRC26.25 antibodies were assessed, including modifications to alter glycosylation, charge, and antibody interaction with antigen. An optimized form of CAP256-VRC26.25 was identified with K100 mA mutation in the HCDR3 and “LS” mutations in the heavy chain constant region. This optimized form of CAP256-VRC26.25 was termed CAP256V2LS. The amino acid sequences of the CAP256V2LS V_{H} and VL are provided as SEQ ID NOs: 1 and 2, respectively. The amino acid sequences of the CAP256V2LS heavy and light chains are provided as SEQ ID NOs: 14 and 55, respectively.

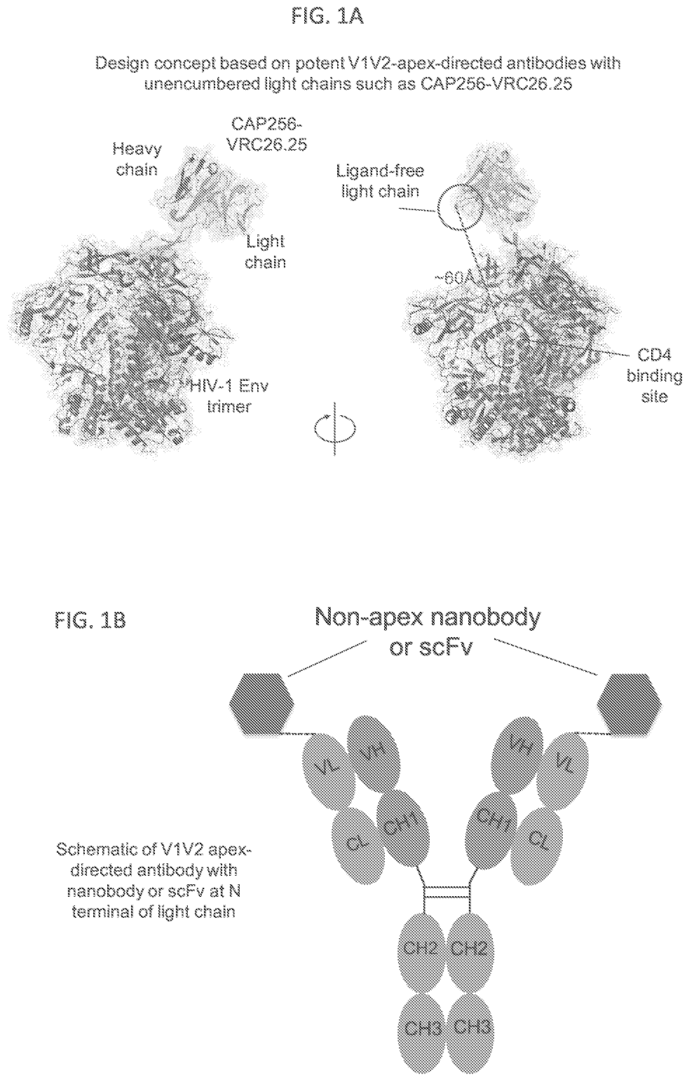

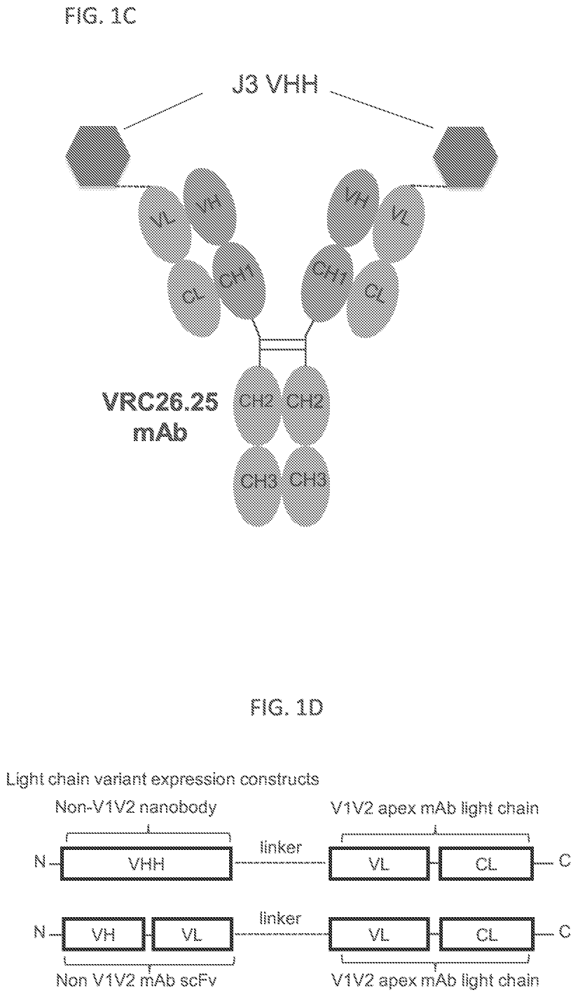

The published structure of the super-potent antibody CAP256-VRC26.25 (Doria-Rose et al., J Virol. 2016; 90:76-91) in complex with a prefusion-closed envelope trimer (Gorman et al., Cell Rep. 2020; 31:107488) reveals that the light chain of this antibody does not interact directly with Env, leaving its N terminus free for conjugation (FIGS. 1A, 1B).

We designed a number of bispecific antibodies by genetically fusing the light chain N terminus of CAP256V2LS with nanobody J3 or scFv antibodies 10E8 and VRC51 that target non-V1V2 sites (FIG. SD and Table 1).

TABLE 1
Bispecific antibody design and sequences

| Antibody Name | Heavy chain | Light chain Format | Linker sequence |
|---|---|---|---|
| CAP256V2LS-J3-1 | CAP256V2LS HC | J3_5 aa flexible linker, | ggsgg |
| | SEQ ID NO: 14 | 5 aa_CAP256V2LS LC | SEQ ID NO: 50 |
| | | SEQ ID NO: 27 | |
| CAP256V2LS-J3-2 | CAP256V2LS HC | J3_flexible linker, | ggsggggsgg |
| | SEQ ID NO: 14 | 10 aa_CAP256V2LS LC | SEQ ID NO: 51 |
| | | SEQ ID NO: 28 | |
| CAP256V2LS-J3-3 | CAP256V2LS HC | J3_flexible linker, | ggsggggsggggsgg |
| | SEQ ID NO: 14 | 15 aa_CAP256V2LS LC | SEQ ID NO: 16 |
| | | SEQ ID NO: 15 | |
| CAP256V2LS-J3-4 | CAP256V2LS HC | J3_1xhuman IgG1H | Dktht |
| | SEQ ID NO: 14 | hinge_CAP256V2LS LC | SEQ ID NO: 52 |
| | | SEQ ID NO: 29 | |
| CAP256V2LS-J3-5 | CAP256V2LS HC | J3_2xhuman IgG1H | dkthtgdktht |
| | SEQ ID NO: 14 | hinge_CAP256V2LS LC | SEQ ID NO: 53 |
| | | SEQ ID NO: 30 | |
| CAP256V2LS-J3-6 | CAP256V2LS HC | J3_3xhuman IgG1H | dkthtgdkthtgdktht |
| | SEQ ID NO: 14 | hinge_CAP256V2LS LC | SEQ ID NO: 54 |
| | | SEQ ID NO: 31 | |
| CAP256V2LS L-5ln- | CAP256V2LS HC | 10E8v4scFv_5 aa flexible linker, | Ggsgg |
| 10E8v4scFv | SEQ ID NO: 14 | 5 aa_CAP256V2LS LC | SEQ ID NO: 50 |
| | | SEQ ID NO: 32 | |
| CAP256V2LS L-10ln- | CAP256V2LS HC | 10E8v4scFv_flexible linker, | ggsggggsgg |
| 10E8v4scFv | SEQ ID NO: 14 | 10 aa_CAP256V2LS LC | SEQ ID NO: 51 |
| | | SEQ ID NO: 33 | |
| CAP256V2LS L-15ln- | CAP256V2LS HC | 10E8v4scFv_flexible linker, | ggsggggsggggsgg |
| 10E8v4scFv | SEQ ID NO: 14 | 15 aa_CAP256V2LS LC | SEQ ID NO: 16 |
| | | SEQ ID NO: 34 | |
| CAP256V2LS L- | CAP256V2LS HC | 10E8v4scFv_1xhuman IgG1H | dktht |
| 1xhinge-10E8v4scFv | SEQ ID NO: 14 | hinge_CAP256V2LS LC | SEQ ID NO: 52 |
| | | SEQ ID NO: 35 | |
| CAP256V2LS L- | CAP256V2LS HC | 10E8v4scFv_2xhuman IgG1H | dkthtgdktht |
| 2xhinge-10E8v4scFv | SEQ ID NO: 14 | hinge_CAP256V2LS LC | SEQ ID NO: 53 |
| | | SEQ ID NO: 36 | |
| CAP256V2LS L- | CAP256V2LS HC | 10E8v4scFv_3xhuman IgG1H | dkthtgdkthtgdktht |
| 3xhinge-10E8v4scFv | SEQ ID NO: 14 | hinge_CAP256V2LS LC | SEQ ID NO: 54 |
| | | SEQ ID NO: 37 | |
| CAP256V2LS L-5ln- | CAP256V2LS HC | VRC01scFv_5 aa flexible linker, | ggsgg |
| VRC01scFv-v4 | SEQ ID NO: 14 | 5 aa_CAP256V2LS LC | SEQ ID NO: 50 |
| | | SEQ ID NO: 38 | |
| CAP256V2LS L-10ln- | CAP256V2LS HC | VRC01scFv_flexible linker, | ggsggggsgg |
| VRC01scFv-v4 | SEQ ID NO: 14 | 10 aa_CAP256V2LS LC | SEQ ID NO: 51 |
| | | SEQ ID NO: 39 | |
| CAP256V2LS L-15ln- | CAP256V2LS HC | VRC01scFv_flexible linker, | ggsggggsggggsgg |
| VRC01scFv-v4 | SEQ ID NO: 14 | 15 aa_CAP256V2LS LC | SEQ ID NO: 16 |
| | | SEQ ID NO: 40 | |
| CAP256V2LS L- | CAP256V2LS HC | VRC01scFv_1xhuman IgG1H | dktht |
| 1xhinge-VRC01scFv- | SEQ ID NO: 14 | hinge_CAP256V2LS LC | SEQ ID NO: 52 |
| v4 | | SEQ ID NO: 41 | |
| CAP256V2LS L- | CAP256V2LS HC | VRC01scFv_2xhuman IgG1H | dkthtgdktht |
| 2xhinge-VRC01scFv- | SEQ ID NO: 14 | hinge_CAP256V2LS LC | SEQ ID NO: 53 |
| v4 | | SEQ ID NO: 42 | |
| CAP256V2LS L- | CAP256V2LS HC | VRC01scFv_3xhuman IgG1H | dkthtgdkthtgdktht |
| 3xhinge-VRC01scFv- | SEQ ID NO: 14 | hinge_CAP256V2LS LC | SEQ ID NO: 54 |
| v4 | | SEQ ID NO: 43 | |

CAP256V2LS L-J3-1 LC (SEQ ID NO: 27)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggQSVLTQPPSVSAAPGQKV
TISCSGNTSNIGNNFVSWYQQRPGRAPQLLIYETDKRPSGIPDRFSASKSGTSGTLAITGLQTGDEADYYCA
TWAASLSSARVFGTGTQVIVLGQPKVNPTVTLFPPSSEELQANKATLVCLISDFYPGAVTVAWKADSSPVKA
GVETTTPSKQSNNKYAASSYLSLTPEQWKSHRSYSCQVTHEGSTVEKTVAPTECS
CAP256V2LS L-J3-2 LC (SEQ ID NO: 28)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggggsggQSVLTQPPSVSAA
PGQKVTISCSGNTSNIGNNFVSWYQQRPGRAPQLLIYETDKRPSGIPDRFSASKSGTSGTLAITGLQTGDEA
DYYCATWAASLSSARVFGTGTQVIVLGQPKVNPTVTLFPPSSEELQANKATLVCLISDFYPGAVTVAWKADS
SPVKAGVETTTPSKQSNNKYAASSYLSLTPEQWKSHRSYSCQVTHEGSTVEKTVAPTECS
CAP256V2LS L-J3-3 LC (SEQ ID NO: 15)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggggsggggsggQSVLTQPP
SVSAAPGQKVTISCSGNTSNIGNNFVSWYQQRPGRAPQLLIYETDKRPSGIPDRFSASKSGTSGTLAITGLQ
TGDEADYYCATWAASLSSARVFGTGTQVIVLGQPKVNPTVTLFPPSSEELQANKATLVCLISDFYPGAVTVA
WKADSSPVKAGVETTTPSKQSNNKYAASSYLSLTPEQWKSHRSYSCQVTHEGSTVEKTVAPTECS
CAP256V2LS L-J3-4 LC (SEQ ID NO: 29)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSdkthtQSVLTQPPSVSAAPGQKV
TISCSGNTSNIGNNFVSWYQQRPGRAPQLLIYETDKRPSGIPDRFSASKSGTSGTLAITGLQTGDEADYYCA
TWAASLSSARVFGTGTQVIVLGQPKVNPTVTLFPPSSEELQANKATLVCLISDFYPGAVTVAWKADSSPVKA
GVETTTPSKQSNNKYAASSYLSLTPEQWKSHRSYSCQVTHEGSTVEKTVAPTECS
CAP256V2LS L-J3-5 LC (SEQ ID NO: 30)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSdkthtgdkthtQSVLTQPPSVSA
APGQKVTISCSGNTSNIGNNFVSWYQQRPGRAPQLLIYETDKRPSGIPDRFSASKSGTSGTLAITGLQTGDE
ADYYCATWAASLSSARVFGTGTQVIVLGQPKVNPTVTLFPPSSEELQANKATLVCLISDFYPGAVTVAWKAD
SSPVKAGVETTTPSKQSNNKYAASSYLSLTPEQWKSHRSYSCQVTHEGSTVEKTVAPTECS
CAP256V2LS L-J3-6 LC (SEQ ID NO: 31)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSdkthtgdkthtgdkthtQSVLTQ
PPSVSAAPGQKVTISCSGNTSNIGNNFVSWYQQRPGRAPQLLIYETDKRPSGIPDRFSASKSGTSGTLAITG
LQTGDEADYYCATWAASLSSARVFGTGTQVIVLGQPKVNPTVTLFPPSSEELQANKATLVCLISDFYPGAVT
VAWKADSSPVKAGVETTTPSKQSNNKYAASSYLSLTPEQWKSHRSYSCQVTHEGSTVEKTVAPTECS
CAP256V2LS L-5ln-10E8v4scFv LC (SEQ ID NO: 32)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSGGGGSGGGGSGGG
GSSELTQDPAVSVALKQTVTITCRGDSLRSHYASWYQKKPGQAPVLLFYGKNNRPSGIPDRFSGSASGNRAS
LTITGAQAEDEADYYCSSRDKSGSRLSVFGGGTKLTVggsggQSVLTQPPSVSAAPGQKVTISCSGNTSNIG
NNFVSWYQQRPGRAPQLLIYETDKRPSGIPDRFSASKSGTSGTLAITGLQTGDEADYYCATWAASLSSARVE
GTGTQVIVLGQPKVNPTVTLFPPSSEELQANKATLVCLISDFYPGAVTVAWKADSSPVKAGVETTTPSKQSN
NKYAASSYLSLTPEQWKSHRSYSCQVTHEGSTVEKTVAPTECS
CAP256V2LS L-10ln-10E8v4scFv LC (SEQ ID NO: 33)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSGGGGSGGGGSGGG
GSSELTQDPAVSVALKQTVTITCRGDSLRSHYASWYQKKPGQAPVLLFYGKNNRPSGIPDRFSGSASGNRAS
LTITGAQAEDEADYYCSSRDKSGSRLSVFGGGTKLTVggsggggsggQSVLTQPPSVSAAPGQKVTISCSGN
TSNIGNNFVSWYQQRPGRAPQLLIYETDKRPSGIPDRFSASKSGTSGTLAITGLQTGDEADYYCATWAASLS
SARVFGTGTQVIVLGQPKVNPTVTLFPPSSEELQANKATLVCLISDFYPGAVTVAWKADSSPVKAGVETTTP
SKQSNNKYAASSYLSLTPEQWKSHRSYSCQVTHEGSTVEKTVAPTECS
CAP256V2LS L-15ln-10E8v4scFv LC (SEQ ID NO: 34)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSGGGGSGGGGSGGG
GSSELTQDPAVSVALKQTVTITCRGDSLRSHYASWYQKKPGQAPVLLFYGKNNRPSGIPDRFSGSASGNRAS
LTITGAQAEDEADYYCSSRDKSGSRLSVFGGGTKLTVggsggggsggggsggQSVLTQPPSVSAAPGQKVTI
SCSGNTSNIGNNFVSWYQQRPGRAPQLLIYETDKRPSGIPDRFSASKSGTSGTLAITGLQTGDEADYYCATW
AASLSSARVFGTGTQVIVLGQPKVNPTVTLFPPSSEELQANKATLVCLISDFYPGAVTVAWKADSSPVKAGV
ETTTPSKQSNNKYAASSYLSLTPEQWKSHRSYSCQVTHEGSTVEKTVAPTECS
CAP256V2LS L-1xhinge-10E8v4scFv LC (SEQ ID NO: 35)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSGGGGSGGGGSGGG
GSSELTQDPAVSVALKQTVTITCRGDSLRSHYASWYQKKPGQAPVLLFYGKNNRPSGIPDRFSGSASGNRAS
LTITGAQAEDEADYYCSSRDKSGSRLSVFGGGTKLTVdkthtQSVLTQPPSVSAAPGQKVTISCSGNTSNIG
NNFVSWYQQRPGRAPQLLIYETDKRPSGIPDRFSASKSGTSGTLAITGLQTGDEADYYCATWAASLSSARVF
GTGTQVIVLGQPKVNPTVTLFPPSSEELQANKATLVCLISDFYPGAVTVAWKADSSPVKAGVETTTPSKQSN
NKYAASSYLSLTPEQWKSHRSYSCQVTHEGSTVEKTVAPTECS
CAP256V2LS L-2xhinge-10E8v4scFv LC (SEQ ID NO: 36)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSGGGGSGGGGSGGG
GSSELTQDPAVSVALKQTVTITCRGDSLRSHYASWYQKKPGQAPVLLFYGKNNRPSGIPDRFSGSASGNRAS
LTITGAQAEDEADYYCSSRDKSGSRLSVFGGGTKLTVdkthtgdkthtQSVLTQPPSVSAAPGQKVTISCSG
NTSNIGNNFVSWYQQRPGRAPQLLIYETDKRPSGIPDRFSASKSGTSGTLAITGLQTGDEADYYCATWAASL
SSARVFGTGTQVIVLGQPKVNPTVTLFPPSSEELQANKATLVCLISDFYPGAVTVAWKADSSPVKAGVETTT
PSKQSNNKYAASSYLSLTPEQWKSHRSYSCQVTHEGSTVEKTVAPTECS
CAP256V2LS L-3xhinge-10E8v4scFv LC (SEQ ID NO: 37)
EVRLVESGGGLVKPGGSLRLSCSASGFDFDNAWMTWVRQPPGKGLEWVGRITGPGEGWSVDYAESVKGRFTI
SRDNTKNTLYLEMNNVRTEDTGYYFCARTGKYYDFWSGYPPGEEYFQDWGQGTLVIVSSGGGGSGGGGSGGG
GSSELTQDPAVSVALKQTVTITCRGDSLRSHYASWYQKKPGQAPVLLFYGKNNRPSGIPDRFSGSASGNRAS
LTITGAQAEDEADYYCSSRDKSGSRLSVFGGGTKLTVdkthtgdkthtgdkthtQSVLTQPPSVSAAPGQKV
TISCSGNTSNIGNNFVSWYQQRPGRAPQLLIYETDKRPSGIPDRFSASKSGTSGTLAITGLQTGDEADYYCA
TWAASLSSARVFGTGTQVIVLGQPKVNPTVTLFPPSSEELQANKATLVCLISDFYPGAVTVAWKADSSPVKA
GVETTTPSKQSNNKYAASSYLSLTPEQWKSHRSYSCQVTHEGSTVEKTVAPTECS
CAP256V2LS L-5ln-VRC01scFv-v4 LC (SEQ ID NO: 38)
QVQLVQSGGQMKKPGESMRISCRASGYEFIDCTLNWIRLAPGKRPEWMGWLKPRGGAVNYARPLQGRVTMTR
DVYSDTAFLELRSLTVDDTAVYFCTRGKNCDYNWDFEHWGRGTPVIVSSGGGGSGGGGSGGGGSLTQSPGTL
SLSPGETAIISCRTSQYGSLAWYQQRPGQAPRLVIYSGSTRAAGIPDRFSGSRWGPDYNLTISNLESGDFGV
YYCQQYEFFGQGTKVQVggsggQSVLTQPPSVSAAPGQKVTISCSGNTSNIGNNFVSWYQQRPGRAPQLLIY
ETDKRPSGIPDRFSASKSGTSGTLAITGLQTGDEADYYCATWAASLSSARVFGTGTQVIVLGQPKVNPTVTL
FPPSSEELQANKATLVCLISDFYPGAVTVAWKADSSPVKAGVETTTPSKQSNNKYAASSYLSLTPEQWKSHR
SYSCQVTHEGSTVEKTVAPTECS
CAP256V2LS L-10ln-VRC01scFv-v4 LC (SEQ ID NO: 39)
QVQLVQSGGQMKKPGESMRISCRASGYEFIDCTLNWIRLAPGKRPEWMGWLKPRGGAVNYARPLQGRVTMTR
DVYSDTAFLELRSLTVDDTAVYFCTRGKNCDYNWDFEHWGRGTPVIVSSGGGGSGGGGSGGGGSLTQSPGTL
SLSPGETAIISCRTSQYGSLAWYQQRPGQAPRLVIYSGSTRAAGIPDRFSGSRWGPDYNLTISNLESGDFGV
YYCQQYEFFGQGTKVQVggsggggsggQSVLTQPPSVSAAPGQKVTISCSGNTSNIGNNFVSWYQQRPGRAP
QLLIYETDKRPSGIPDRFSASKSGTSGTLAITGLQTGDEADYYCATWAASLSSARVFGTGTQVIVLGQPKVN
PTVTLFPPSSEELQANKATLVCLISDFYPGAVTVAWKADSSPVKAGVETTTPSKQSNNKYAASSYLSLTPEQ
WKSHRSYSCQVTHEGSTVEKTVAPTECS
CAP256V2LS L-15ln-VRC01scFv-v4 LC (SEQ ID NO: 40)
QVQLVQSGGQMKKPGESMRISCRASGYEFIDCTLNWIRLAPGKRPEWMGWLKPRGGAVNYARPLQGRVTMTR
DVYSDTAFLELRSLTVDDTAVYFCTRGKNCDYNWDFEHWGRGTPVIVSSGGGGSGGGGSGGGGSLTQSPGTL
SLSPGETAIISCRTSQYGSLAWYQQRPGQAPRLVIYSGSTRAAGIPDRFSGSRWGPDYNLTISNLESGDFGV
YYCQQYEFFGQGTKVQVggsggggsggggsggQSVLTQPPSVSAAPGQKVTISCSGNTSNIGNNFVSWYQQR
PGRAPQLLIYETDKRPSGIPDRFSASKSGTSGTLAITGLQTGDEADYYCATWAASLSSARVEGTGTQVIVLG
QPKVNPTVTLFPPSSEELQANKATLVCLISDFYPGAVTVAWKADSSPVKAGVETTTPSKQSNNKYAASSYLS
LTPEQWKSHRSYSCQVTHEGSTVEKTVAPTECS
CAP256V2LS L-1xhinge-VRC01scFv-v4 LC (SEQ ID NO: 41)
QVQLVQSGGQMKKPGESMRISCRASGYEFIDCTLNWIRLAPGKRPEWMGWLKPRGGAVNYARPLQGRVTMTR
DVYSDTAFLELRSLTVDDTAVYFCTRGKNCDYNWDFEHWGRGTPVIVSSGGGGSGGGGSGGGGSLTQSPGTL
SLSPGETAIISCRTSQYGSLAWYQQRPGQAPRLVIYSGSTRAAGIPDRFSGSRWGPDYNLTISNLESGDFGV
YYCQQYEFFGQGTKVQVdkthtQSVLTQPPSVSAAPGQKVTISCSGNTSNIGNNFVSWYQQRPGRAPQLLIY
ETDKRPSGIPDRFSASKSGTSGTLAITGLQTGDEADYYCATWAASLSSARVFGTGTQVIVLGQPKVNPTVTL
FPPSSEELQANKATLVCLISDFYPGAVTVAWKADSSPVKAGVETTTPSKQSNNKYAASSYLSLTPEQWKSHR
SYSCQVTHEGSTVEKTVAPTECS
CAP256V2LS L-2xhinge-VRC01scFv-v4 LC (SEQ ID NO: 42)
QVQLVQSGGQMKKPGESMRISCRASGYEFIDCTLNWIRLAPGKRPEWMGWLKPRGGAVNYARPLQGRVTMTR
DVYSDTAFLELRSLTVDDTAVYFCTRGKNCDYNWDFEHWGRGTPVIVSSGGGGSGGGGSGGGGSLTQSPGTL
SLSPGETAIISCRTSQYGSLAWYQQRPGQAPRLVIYSGSTRAAGIPDRFSGSRWGPDYNLTISNLESGDFGV
YYCQQYEFFGQGTKVQVdkthtdkthtQSVLTQPPSVSAAPGQKVTISCSGNTSNIGNNFVSWYQQRPGRAP
QLLIYETDKRPSGIPDRFSASKSGTSGTLAITGLQTGDEADYYCATWAASLSSARVFGTGTQVIVLGQPKVN
PTVTLFPPSSEELQANKATLVCLISDFYPGAVTVAWKADSSPVKAGVETTTPSKQSNNKYAASSYLSLTPEQ
WKSHRSYSCQVTHEGSTVEKTVAPTECS
CAP256V2LS L-3xhinge-VRC01scFv-v4 LC (SEQ ID NO: 43)
QVQLVQSGGQMKKPGESMRISCRASGYEFIDCTLNWIRLAPGKRPEWMGWLKPRGGAVNYARPLQGRVTMTR
DVYSDTAFLELRSLTVDDTAVYFCTRGKNCDYNWDFEHWGRGTPVIVSSGGGGSGGGGSGGGGSLTQSPGTL
SLSPGETAIISCRTSQYGSLAWYQQRPGQAPRLVIYSGSTRAAGIPDRFSGSRWGPDYNLTISNLESGDFGV
YYCQQYEFFGQGTKVQVdkthtdkthtdkthtQSVLTQPPSVSAAPGQKVTISCSGNTSNIGNNFVSWYQQR
PGRAPQLLIYETDKRPSGIPDRFSASKSGTSGTLAITGLQTGDEADYYCATWAASLSSARVFGTGTQVIVLG
QPKVNPTVTLFPPSSEELQANKATLVCLISDFYPGAVTVAWKADSSPVKAGVETTTPSKQSNNKYAASSYLS
LTPEQWKSHRSYSCQVTHEGSTVEKTVAPTECS

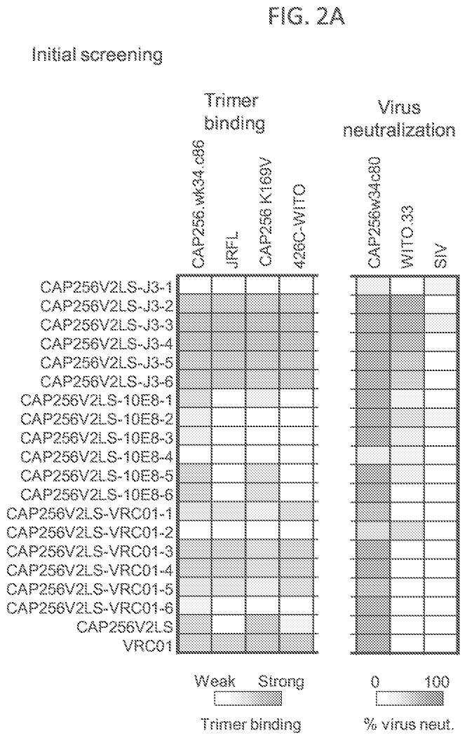

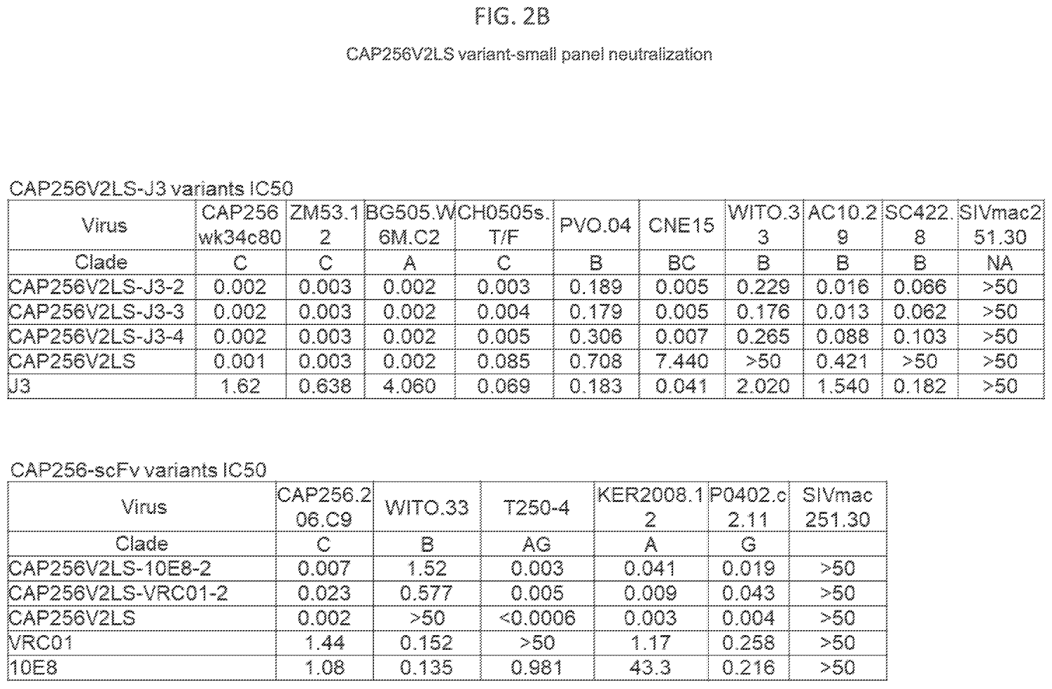

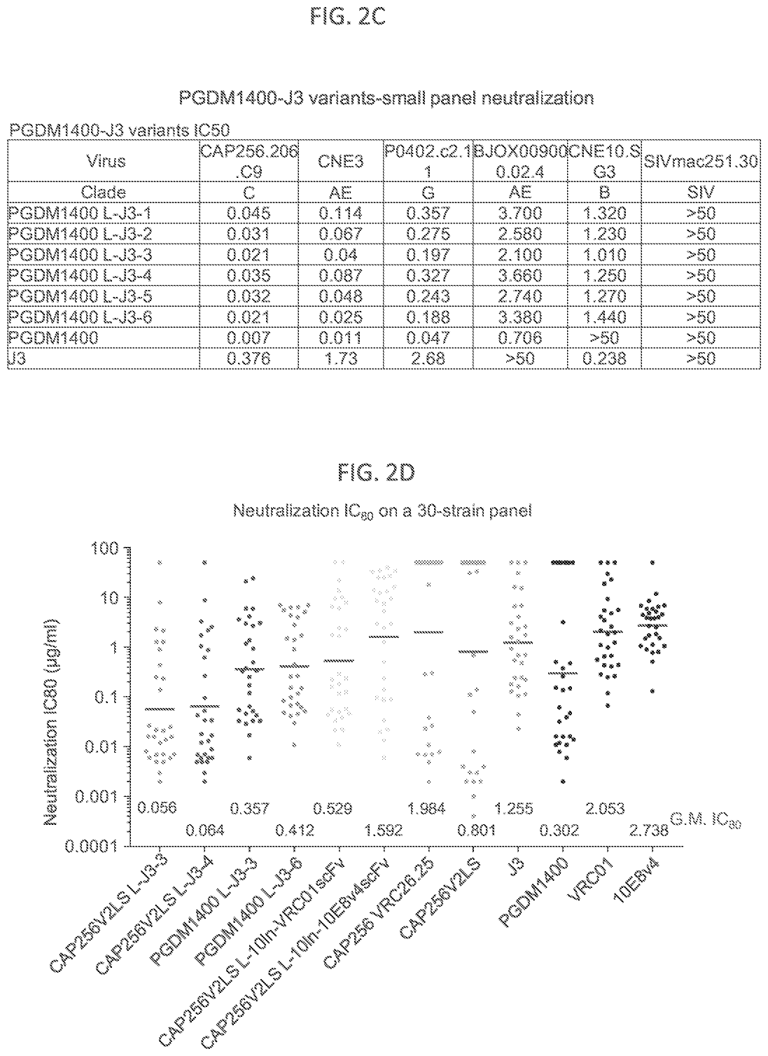

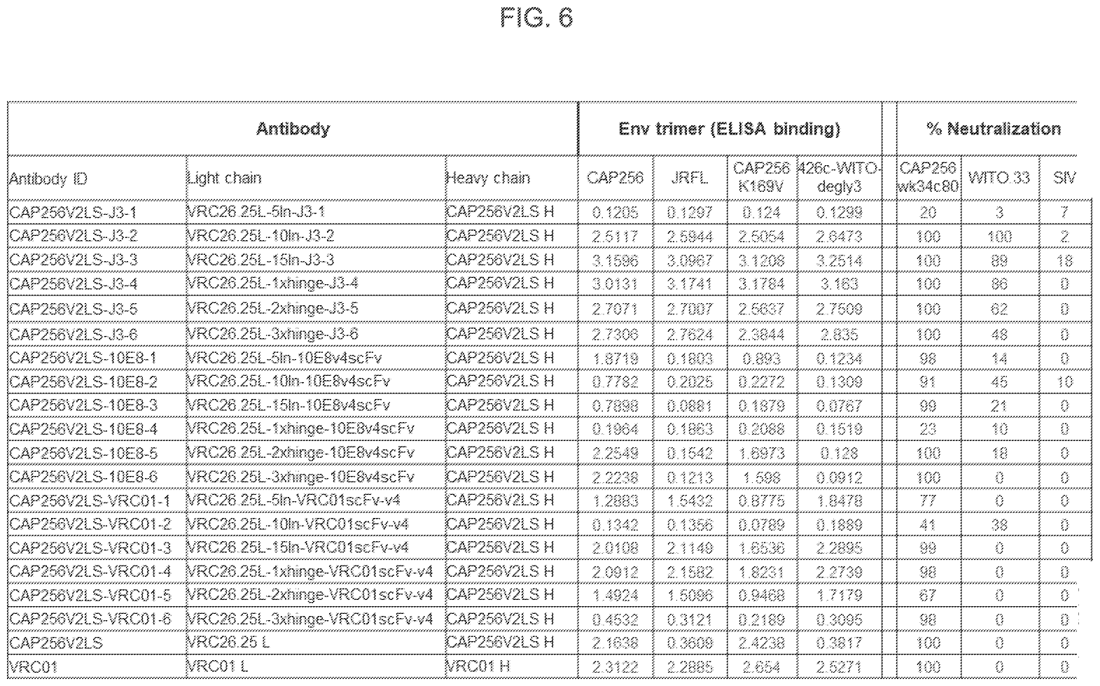

We synthesized, expressed, and tested these light-chain variants of CAP256V2LS for binding to soluble Env trimers stabilized in the prefusion-closed confirmation by automated design and consensus repair (Rawi et al., Cell Rep. 2020;33(8):108432). Tested strains included CAP256 (from clade C), JRFL (B), 426C-WITO (B), as well as a K169V variant of CAP256 with reduced binding of CAP256-VRC26.25; we also tested the ability of the light-chain variants to neutralize CAP256 and WITO.33, with simian immunodeficiency virus (SIV) as a negative control (FIGS. 2A, 6). We found several light-chain variants with J3 fusion to bind tightly Env trimers of tested strains and to neutralize both CAP256 and WITO.33, but not SIV. The best J3 variants (CAP256V2LS-J3-2, -3, and -4) used linkers of 2× GGSGG (SEQ ID NO: 51), 3× GGSGG (SEQ ID NO: 16), and 1× DKTHT (SEQ ID NO: 52), respectively (FIG. 6 and Table 1), and we further tested these on a nine-strain panel, including WITO.33 and SC422.8, which were resistant to CAP256-VRC26.25, but moderately sensitive to J3. Notably, the neutralization potency of CAP256V2LS-J3-2 and CAP256V2LS-J3-3, whose linkers were 10—and 15-residues, respectively, was ˜10-fold better against WITO.33 and 3-fold against SC422.8 than that of J3 (FIG. 2B). Neutralization potencies against strains modestly neutralized by CAP256V2LS were also substantially improved for these two bispecific antibodies, relative to that of both CAP256V2LS and J3. Improved neutralization appeared to be specific to the bispecific antibodies with J3 fusion and was not observed with bispecific antibodies constructed from scFvs of VRC01 or 10E8. We attribute the lower efficacy of scFv fusion to the larger size of scFv versus nanobody; the substantially smaller size of the nanobody likely enables it to reach the CD4 binding site, while simultaneously allows recognition at the V2 apex site (FIG. 2B).

To test if J3 fusion to the N terminus of light chain could improve other V2 apex antibodies, we tried the same strategy with PGDM1400 using six linkers ranging in length from 5 to 15 residues (FIG. 2C, Table 2).

TABLE 2
Bispecific antibody format and sequences

| Antibody Name | Heavy chain | Light chain Format | Linker sequence |
|---|---|---|---|
| PGDM L-J3-1 | CAP256V2LS HC | PGDM VHH_5 aa flexible linker, | ggsgg |
| | SEQ ID NO: 14 | 5 aa_CAP256V2LS LC | SEQ ID NO: 50 |
| | | SEQ ID NO: 44 | |
| PGDM L-J3-2 | CAP256V2LS HC | PGDM VHH_flexible linker, | ggsggggsgg |
| | SEQ ID NO: 14 | 10 aa_CAP256V2LS LC | SEQ ID NO: 51 |
| | | SEQ ID NO: 45 | |
| PGDM L-J3-3 | CAP256V2LS HC | PGDM VHH_flexible linker, | ggsggggsggggsgg |
| | SEQ ID NO: 14 | 15 aa_CAP256V2LS LC | SEQ ID NO: 16 |
| | | SEQ ID NO: 46 | |
| PGDM L-J3-4 | CAP256V2LS HC | PGDM VHH_1xhuman IgG1H | dktht |
| | SEQ ID NO: 14 | hinge_CAP256V2LS LC | SEQ ID NO: 52 |
| | | SEQ ID NO: 47 | |
| PGDM L-J3-5 | CAP256V2LS HC | PGDM VHH_2xhuman IgG1H | dkthtgdktht |
| | SEQ ID NO: 14 | hinge_CAP256V2LS LC | SEQ ID NO: 53 |
| | | SEQ ID NO: 48 | |
| PGDM L-J3-6 | CAP256V2LS HC | PGDM VHH_3xhuman IgG1H | dkthtgdkthtgdktht |
| | SEQ ID NO: 14 | hinge_CAP256V2LS LC | SEQ ID NO: 54 |
| | | SEQ ID NO: 49 | |

PGDM L-J3-1 LC (SEQ ID NO: 44)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggDFVLTQSPHSLSVTPGES
ASISCKSSHSLIHGDRNNYLAWYVQKPGRSPQLLIYLASSRASGVPDRFSGSGSDKDFTLKISRVETEDVGT
YYCMQGRESPWTFGQGTKVDIKTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
PGDM L-J3-2 LC (SEQ ID NO: 45)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggggsggDFVLTQSPHSLSV
TPGESASISCKSSHSLIHGDRNNYLAWYVQKPGRSPQLLIYLASSRASGVPDRFSGSGSDKDFTLKISRVET
EDVGTYYCMQGRESPWTFGQGTKVDIKTVAAPSVFIFPPSDEQLKSGTASVVCLLNNFYPREAKVQWKVDNA
LQSGNSQESVTEQDSKDSTYSLSSTLTLSKADYEKHKVYACEVTHQGLSSPVTKSFNRGEC
PGDM L-J3-3 LC (SEQ ID NO: 46)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSggsggggsggggsggDFVLTQSP
HSLSVTPGESASISCKSSHSLIHGDRNNYLAWYVQKPGRSPQLLIYLASSRASGVPDRFSGSGSDKDFTLKI
SRVETEDVGTYYCMQGRESPWTFGQGTKVDIKTVAAPSVFIFPPSDEQLKSGTASVVCLLNNFYPREAKVQW
KVDNALQSGNSQESVTEQDSKDSTYSLSSTLTLSKADYEKHKVYACEVTHQGLSSPVTKSFNRGEC
PGDM L-J3-4 LC (SEQ ID NO: 47)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSdkthtDFVLTQSPHSLSVTPGES
ASISCKSSHSLIHGDRNNYLAWYVQKPGRSPQLLIYLASSRASGVPDRFSGSGSDKDFTLKISRVETEDVGT
YYCMQGRESPWTFGQGTKVDIKTVAAPSVFIFPPSDEQLKSGTASVVCLLNNFYPREAKVQWKVDNALQSGN
SQESVTEQDSKDSTYSLSSTLTLSKADYEKHKVYACEVTHQGLSSPVTKSFNRGEC
PGDM L-J3-5 LC (SEQ ID NO: 48)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSdkthtgdkthtDFVLTQSPHSLS
VTPGESASISCKSSHSLIHGDRNNYLAWYVQKPGRSPQLLIYLASSRASGVPDRFSGSGSDKDFTLKISRVE
TEDVGTYYCMQGRESPWTFGQGTKVDIKTVAAPSVFIFPPSDEQLKSGTASVVCLLNNFYPREAKVQWKVDN
ALQSGNSQESVTEQDSKDSTYSLSSTLTLSKADYEKHKVYACEVTHQGLSSPVTKSFNRGEC
PGDM L-J3-6 LC (SEQ ID NO: 49)
EVQLVESGGGLVQAGGFLRLSCELRGSIFNQYAMAWFRQAPGKEREFVAGMGAVPHYGEFVKGRFTISRDNA
KSTVYLQMSSLKPEDTAIYFCARSKSTYISYNSNGYDYWGRGTQVTVSSdkthtgdkthtgdkthtDFVLTQ
SPHSLSVTPGESASISCKSSHSLIHGDRNNYLAWYVQKPGRSPQLLIYLASSRASGVPDRFSGSGSDKDETL
KISRVETEDVGTYYCMQGRESPWTFGQGTKVDIKTVAAPSVFIFPPSDEQLKSGTASVVCLLNNFYPREAKV
QWKVDNALQSGNSQESVTEQDSKDSTYSLSSTLTLSKADYEKHKVYACEVTHQGLSSPVTKSFNRGEC

All six of the PGDM1400-J3 variants showed weaker neutralization than either PGDM14fb-23, or J3, suggesting that unlike the CAP256V2LS-J3 fusions, the PGDM1400-J3 variants could not simultaneously recognize both epitopes, likely because the light chain N terminus, the site of attachment for the nanobody, is substantially closer to V2 apex in the case of PGDM1400 versus that of CAP256V2LS.

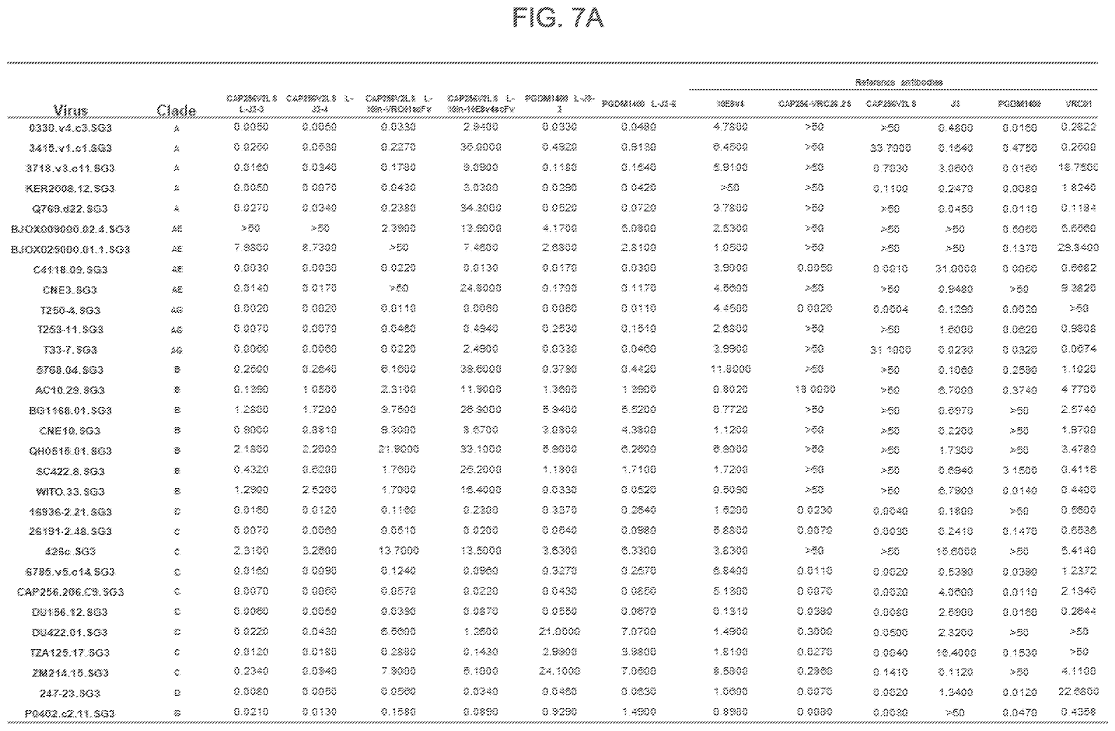

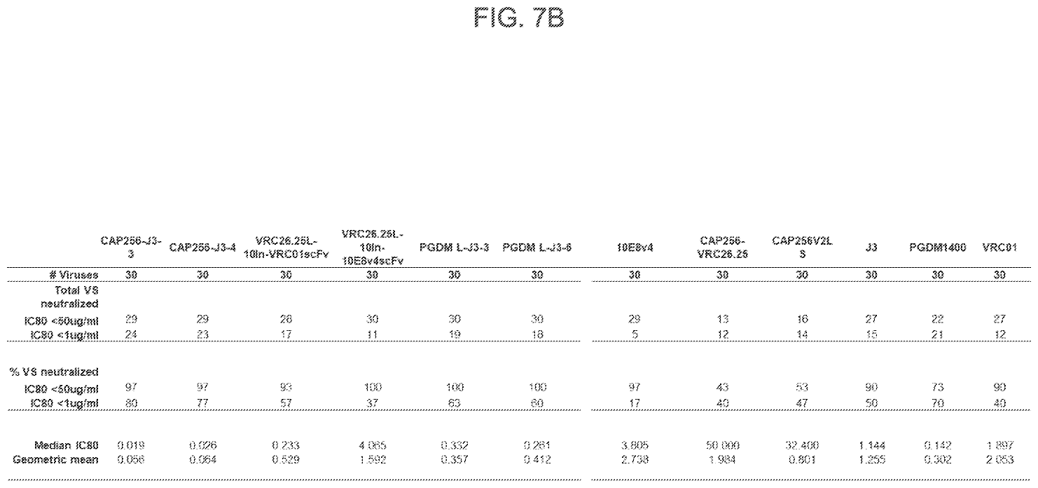

Neutralization assessment on a 30-strain panel showed that several of the tested variants exhibited improved properties, with the CAP256V2LS-J3-3 variant to be the best, slightly better than CAP256V2LS-J3-4 variant, and substantially better than other bispecific antibodies with PGDM1400 or CAP256V2LS with scFvs (FIGS. 2D, 7A-7B).

Structure of CAP256V2LS-J3-3 in Complex with BG505 DS-SOSIP.664

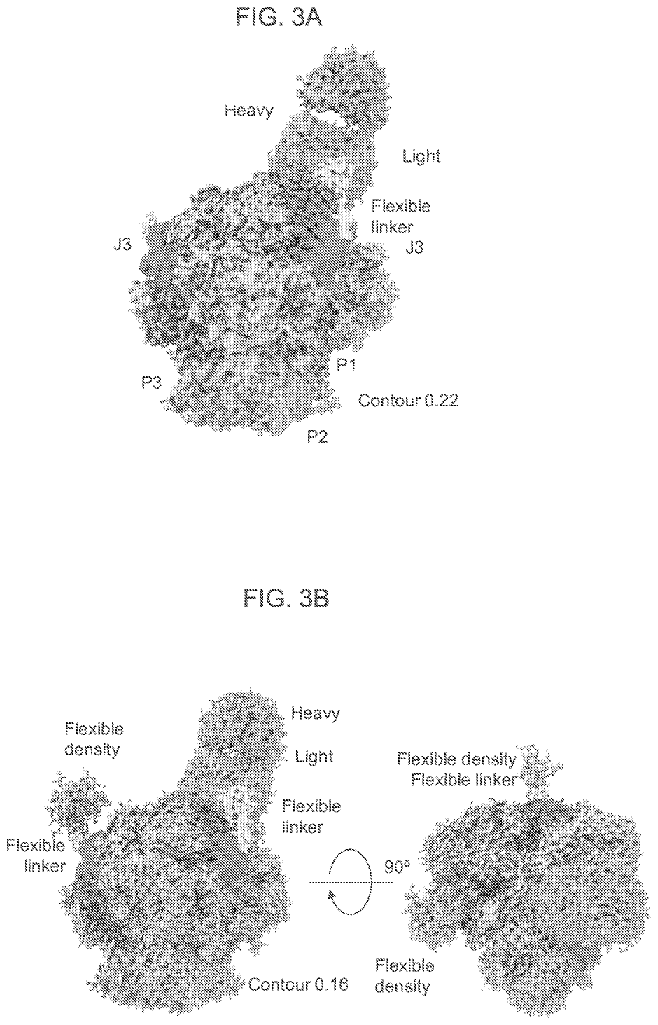

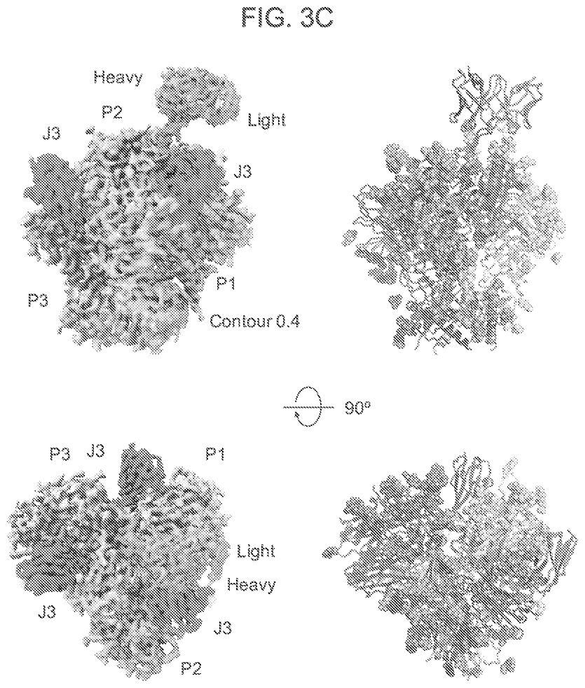

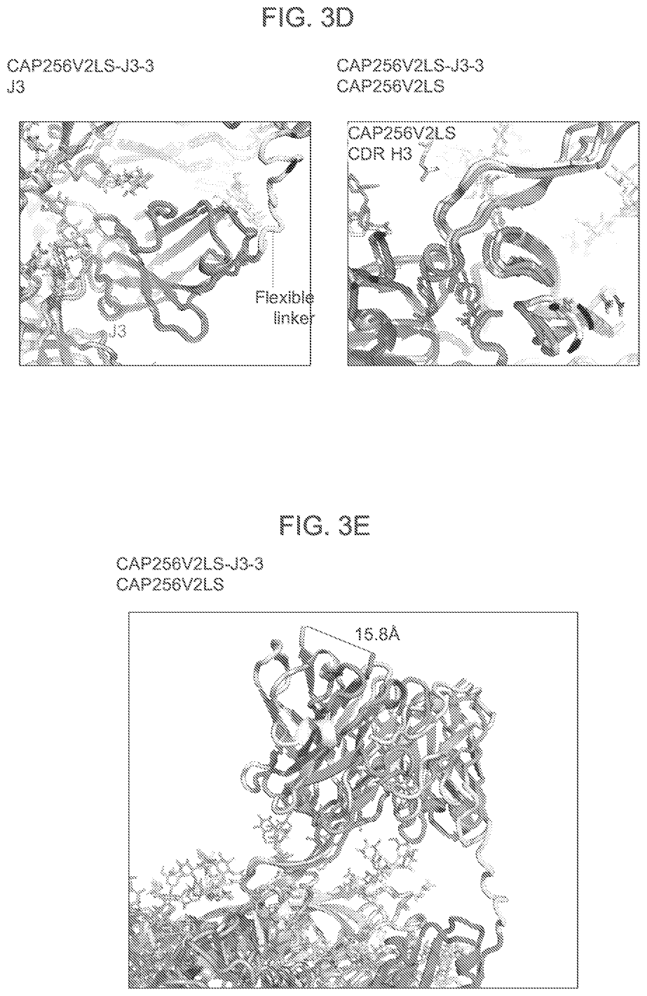

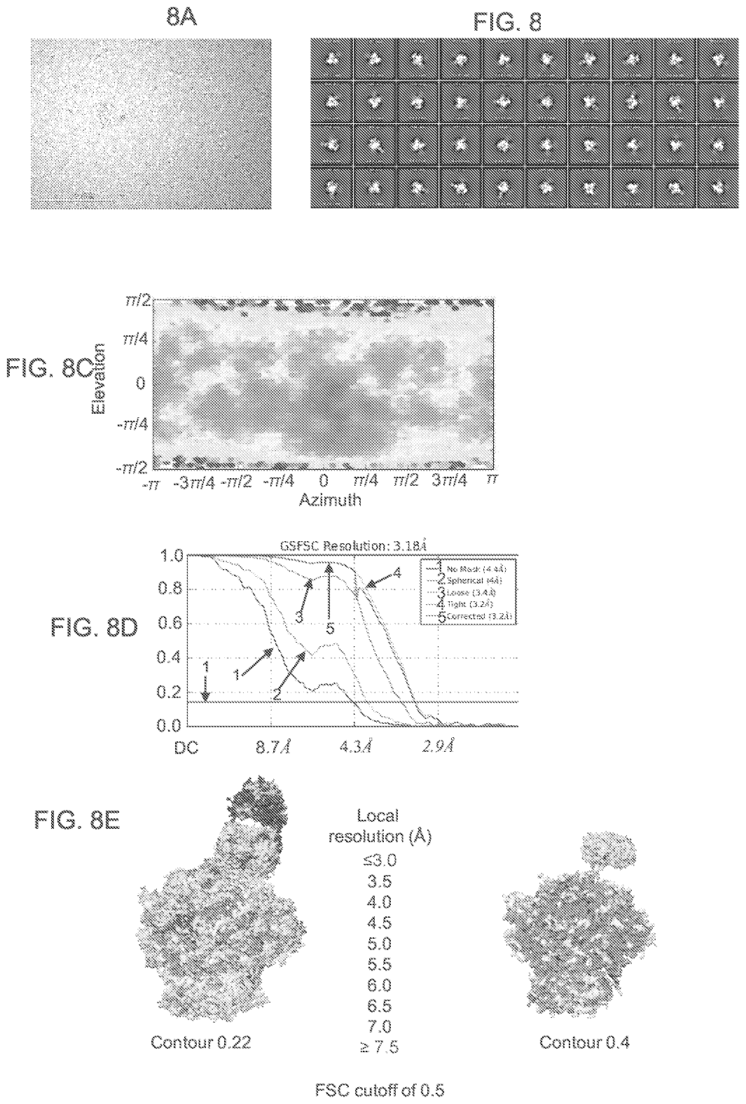

To elucidate the mechanism of the synergistic binding and neutralization of the CAP256V2LS-J3 bispecific antibody, we determined the cryo-EM structure of CAP256V2LS-J3-3 antigen-binding fragment (Fab) in complex with BG505 DS-SOSIP.644 trimer (Gulla et al., Vaccine. 2021; 39(25):3379-87; Kwon et al., Nat Struct Mol Biol. 2015; 22(7):522-31) at 3.2 Å resolution (FIGS. 3A, 8; Table 3). We mixed CAP256V2LS-J3-3 with BG505 DS-SOSIP.664 trimer at a 3.5:1 molar ratio for cryo-EM grid preparation. As expected, a single CAP256V2LS Fab was observed to bind at the apex hole formed by the three protomers of the trimer, as observed previously for the CAP256-VRC26.25 Fab in a complex with Env trimer (Gorman et al., Cell Rep. 2020; 31:107488). Clear density was present for the linker connecting CAP256V2LS light chain N terminus to the C terminus of J3 binding at the most proximal CD4-binding site. At each of the other two CD4-binding sites of the trimer, J3 was observed to bind, with weak density extending from its C terminus, but the linked CAP256V2LS was not visible (FIG. 3B). The linker density was mostly missing at a higher contour level (FIG. 3C), indicating that the linker for these two molecules was flexible. At this contour level, the density for the constant domains of CAP256V2LS bound at the trimer apex was also not visible, and only its variable domains could be seen (FIG. 3C). Binding of J3 generally recapitulated the binding mode observed for J3 alone, as did the CDR H3 of CAP256-VRC26.25 (FIG. 3D); however, the variable domain swiveled, so that the C terminus of the heavy chain was displaced almost 16 Å (FIG. 3E).

Overall, the structure confirmed the ability of the CAP256V2LS-J3-3 to bind simultaneously to both V2 apex and CD4-binding site, with two other copies of the bispecific antibody binding through only their J3 moieties to the other two CD4-binding sites of the trimer.

TABLE 3
Cryo-EM data collection and refinement statistics.
VRC26.25-J3 in complex with BG505 DS-SOSIP.664

| | |
|---|---|
| EMDB ID | 29209 |
| PDB ID | 8FIS |

Data collection

| | |
|---|---|
| Microscope | FEI Titan Krios |
| Voltage (kV) | 300 |
| Electron dose (e⁻/Å²) | 51.19 |
| Detector | Gatan K3 |
| Pixel size (Å) | 1.083 |
| Defocus range (μm) | −0.8 to −2.5 |
| Magnification | 81000 |

Reconstruction

| | |
|---|---|
| Software | cryoSparc v3.1 |
| Particles | 118,472 |
| Symmetry | C1 |
| Box size (pix) | 400 |
| Resolution (Å) (FSC_{0.143}) | 3.18 |

Refinement

| | |
|---|---|
| Software | Phenix 1.20 |
| Protein residues | 2357 |
| Chimera CC | 0.81 |
| EMRinger score | 2.50 |

R.m.s. deviations

| | |
|---|---|
| Bond lengths (Å) | 0.005 |
| Bond angles (°) | 0.675 |

Validation

| | |
|---|---|
| Molprobity score | 1.46 |
| Clash score | 2.90 |
| Favored rotamers (%) | 99.5 |

Ramachandran

| | |
|---|---|
| Favored regions (%) | 94.47 |
| Disallowed regions (%) | 0.0 |

* The J3 chain is numbered according to the Kabat numbering scheme and the linked VRC26.25 light chain is numbered according to Kabat but offset by 1000 to adhere to standard PDB file formatting.

### Improved Pharmacokinetics of CAP256.J3LS Achieved by Altering Surface Charge of J3

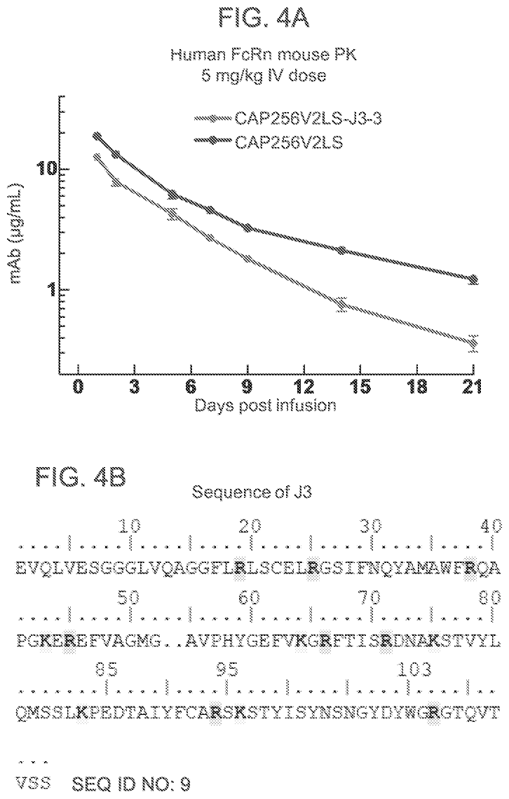

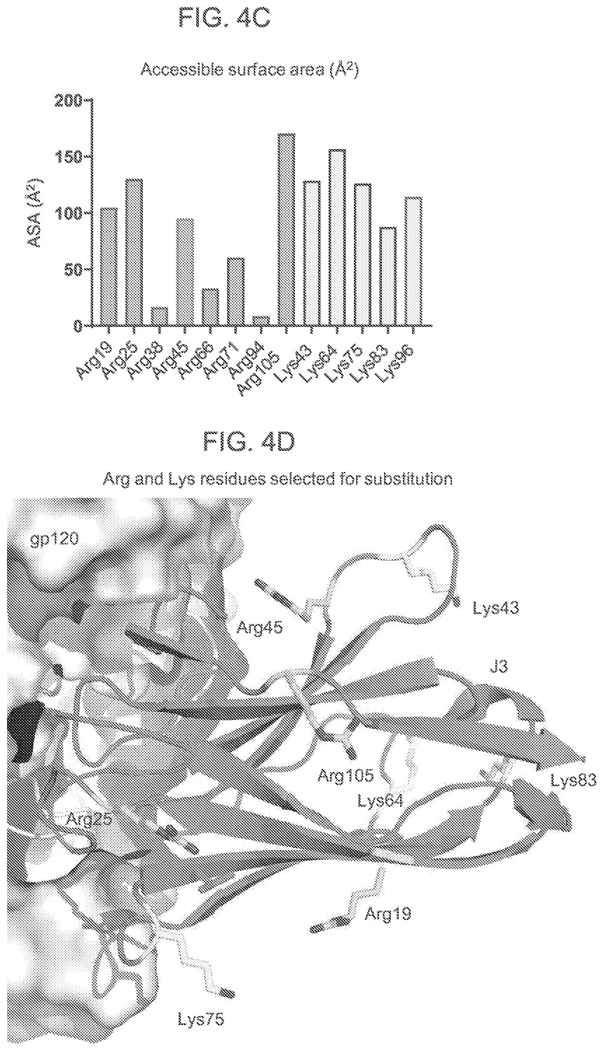

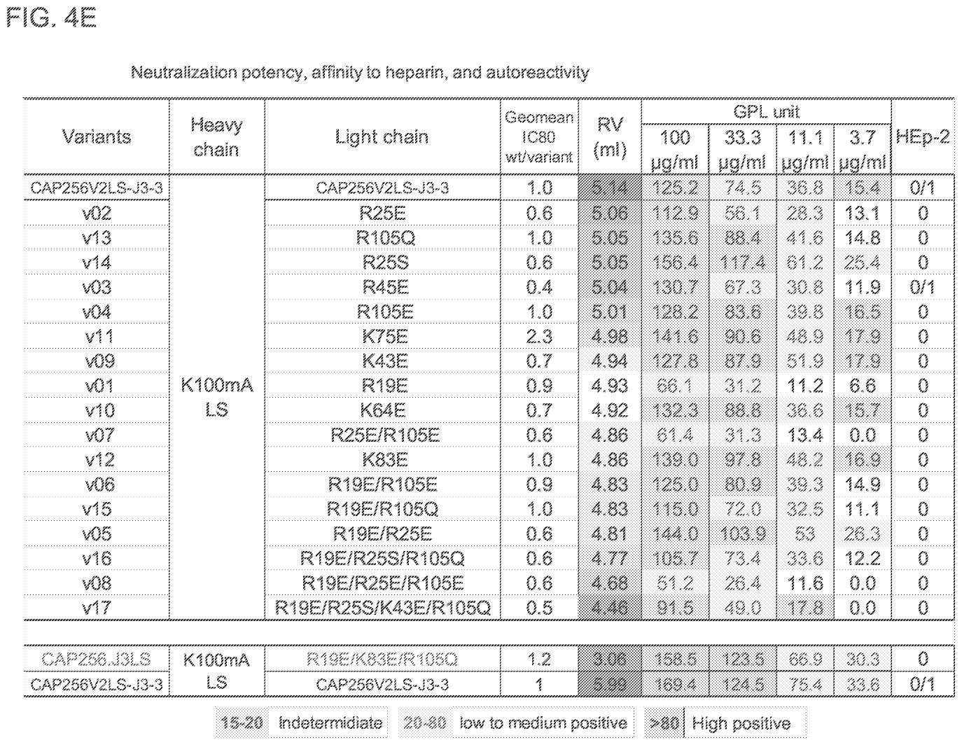

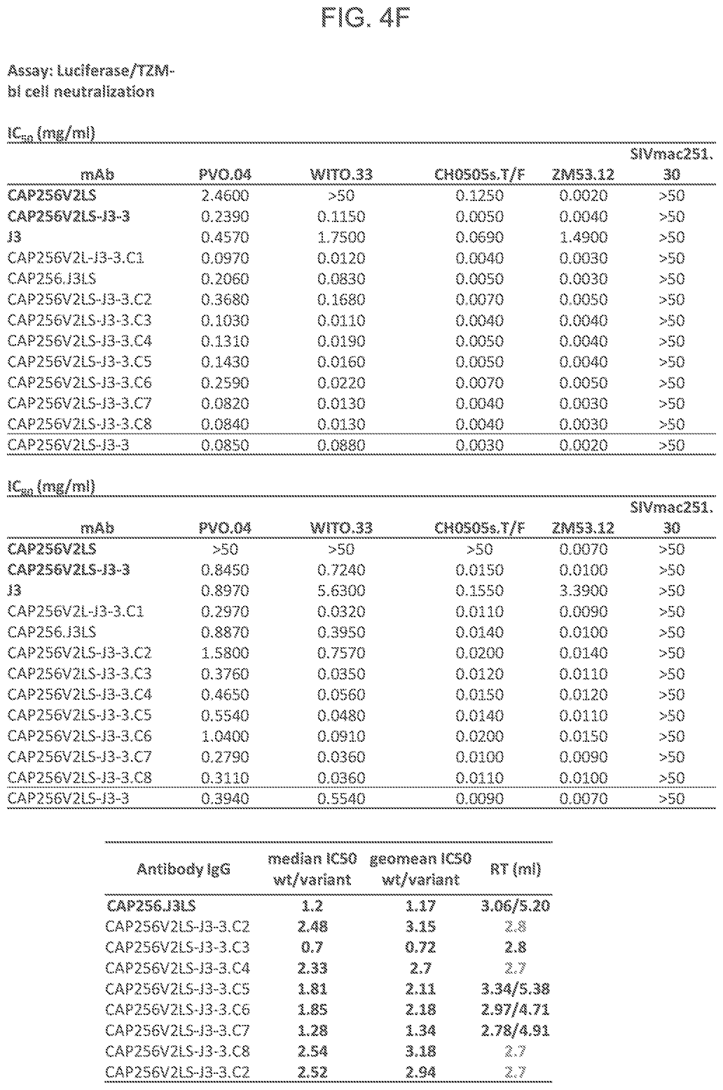

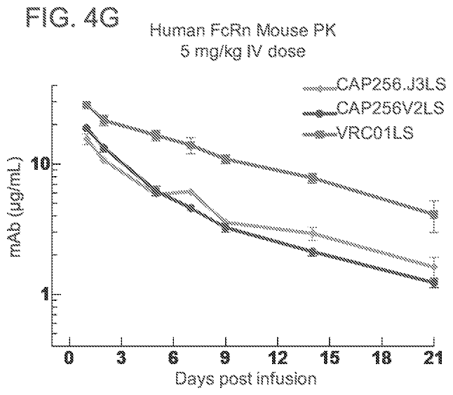

We compared the pharmacokinetics of CAP256V2LS-J3-3 with its parent antibody CAP256V2LS. We observed the bispecific antibody to have reduced half-life relative to CAP256V2LS in neonatal Fc receptor (FcRn)-knock-in mice (FIG. 4A; Table 4).

TABLE 4
Humanized FcRn mice PK data.

| | | | | | Average | | Average | | |
|---|---|---|---|---|---|---|---|---|---|
| | | | | Half- | half- | AUC | AUC | | Average |
| | Dose | | Animal | life | life | [day*{mcg/ | [day*{mcg/ | Clearance | clearance |
| Ab | (mg/kg) | Route | ID | (day) | (day) | mL}] | mL}] | (ml/day/kg) | (ml/day/kg) |
| CAP256V2LS | 5 | IV | 3691 | 5.1 | 6.3 | 128 | 126 | 39.1 | 40.4 |
| | 5 | IV | 3692 | 6.6 | | 152 | | 32.9 | |
| | 5 | IV | 3693 | 6.5 | | 102 | | 49.0 | |
| | 5 | IV | 3694 | 5.9 | | 131 | | 38.2 | |
| | 5 | IV | 3695 | 7.2 | | 117 | | 42.7 | |
| CAP256.J3LS | 5 | IV | 8576 | 7.6 | 7.2 | 124 | 116 | 40.4 | 43.4 |
| | 5 | IV | 8577 | 4.2 | | 100 | | 50.0 | |
| | 5 | IV | 8578 | 7.1 | | 111 | | 45.0 | |
| | 5 | IV | 8579 | 4.6 | | 121 | | 41.3 | |
| | 5 | IV | 8580 | 12.4 | | 124 | | 40.5 | |
| CAP256V2LS-J3-3 | 5 | IV | 3778 | 2.8 | 4.6 | 58 | 70 | 86.0 | 72.3 |
| | 5 | IV | 3779 | 5.8 | | 75 | | 66.9 | |
| | 5 | IV | 3782 | 4.4 | | 75 | | 67.0 | |
| | 5 | IV | 3783 | 3.7 | | 74 | | 67.2 | |
| | 5 | IV | 3784 | 6.1 | | 67 | | 74.7 | |
| VRC01LS | 5 | IV | 2136 | 8.0 | 9.7 | 379 | 289 | 13.2 | 18.7 |
| | 5 | IV | 2137 | 10.0 | | 360 | | 13.9 | |
| | 5 | IV | 2138 | 9.0 | | 225 | | 22.2 | |
| | 5 | IV | 2139 | 7.4 | | 313 | | 16.0 | |
| | 5 | IV | 2140 | 9.7 | | 371 | | 13.5 | |
| | 5 | IV | 2105 | 10.2 | | 222 | | 22.6 | |
| | 5 | IV | 2106 | 11.8 | | 357 | | 14.0 | |
| | 5 | IV | 2125 | 6.6 | | 155 | | 32.4 | |
| | 5 | IV | 2126 | 16.0 | | 260 | | 19.2 | |
| | 5 | IV | 2134 | 8.7 | | 253 | | 19.7 | |

To improve half-life, we sought to decrease the electropositivity of J3 V_{H}H segment of the bispecific antibody by mutating surface-exposed Lys and Arg to Glu or other less positively charged amino acids. We analyzed the surface accessibility of Lys and Arg residues on J3 and mutated the surface-exposed residues one at a time or in combinations (FIGS. 4B-4F). Modified antibodies including the CAP256V2LS-J3-3 heavy chain (SEQ ID NO: 14) paired with VRC26.25L-J3-3 light chain (SEQ ID NO: 15) modified with charge-altering mutations were designed and produced, as shown in Table 5.

TABLE 5
Variant CAP256V2LS-J3-3 bispecific antibodies

| | | Light chain mutations |
|---|---|---|
| | | based on VRC26.25L-J3-3, |
| Antibody Name | Heavy Chain | SEQ ID NO: 15 |
| CAP256V2LS-J3-3 v01 | CAP256V2LS HC | R19E |
| | SEQ ID NO: 14 | |
| CAP256V2LS-J3-3 v02 | CAP256V2LS HC | R25E |
| | SEQ ID NO: 14 | |
| CAP256V2LS-J3-3 v03 | CAP256V2LS HC | R45E |
| | SEQ ID NO: 14 | |
| CAP256V2LS-J3-3 v04 | CAP256V2LS HC | R105E |
| | SEQ ID NO: 14 | |
| CAP256V2LS-J3-3 v05 | CAP256V2LS HC | R19E_R25E |
| | SEQ ID NO: 14 | |
| CAP256V2LS-J3-3 v06 | CAP256V2LS HC | R19E_R105E |
| | SEQ ID NO: 14 | |
| CAP256V2LS-J3-3 v07 | CAP256V2LS HC | R25E_R105E |
| | SEQ ID NO: 14 | |
| CAP256V2LS-J3-3 v08 | CAP256V2LS HC | R19E_R25E_R105E |
| | SEQ ID NO: 14 | |
| CAP256V2LS-J3-3 v09 | CAP256V2LS HC | K43E |
| | SEQ ID NO: 14 | |
| CAP256V2LS-J3-3 v10 | CAP256V2LS HC | K62E |
| | SEQ ID NO: 14 | |
| CAP256V2LS-J3-3 v11 | CAP256V2LS HC | K75E |
| | SEQ ID NO: 14 | |
| CAP256V2LS-J3-3 v12 | CAP256V2LS HC | K83E |
| | SEQ ID NO: 14 | |
| CAP256V2LS-J3-3 v13 | CAP256V2LS HC | R105Q |
| | SEQ ID NO: 14 | |
| CAP256V2LS-J3-3 v14 | CAP256V2LS HC | R25S |
| | SEQ ID NO: 14 | |
| CAP256V2LS-J3-3 v15 | CAP256V2LS HC | R19E R105Q |
| | SEQ ID NO: 14 | |
| CAP256V2LS-J3-3 v16 | CAP256V2LS HC | R19E R25S R105Q |
| | SEQ ID NO: 14 | |
| CAP256V2LS-J3-3 v17 | CAP256V2LS HC | R19E R25S K43E, R105Q |
| | SEQ ID NO: 14 | |
| CAP256.J3LS | CAP256V2LS HC | R19E K83E R105Q |
| | SEQ ID NO: 14 | (SEQ ID NO: 17) |

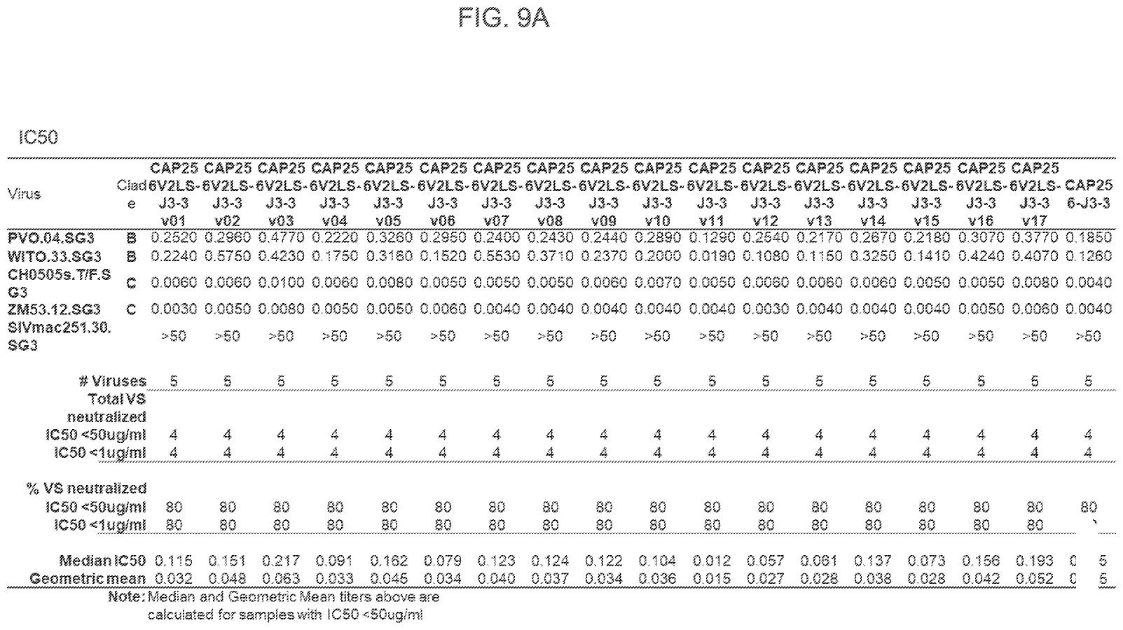

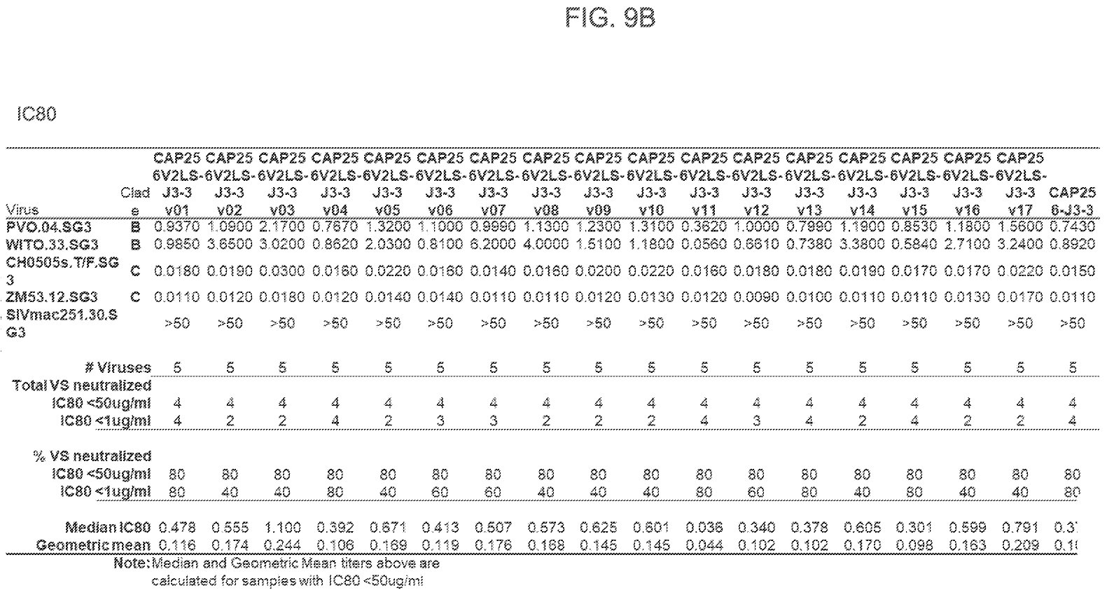

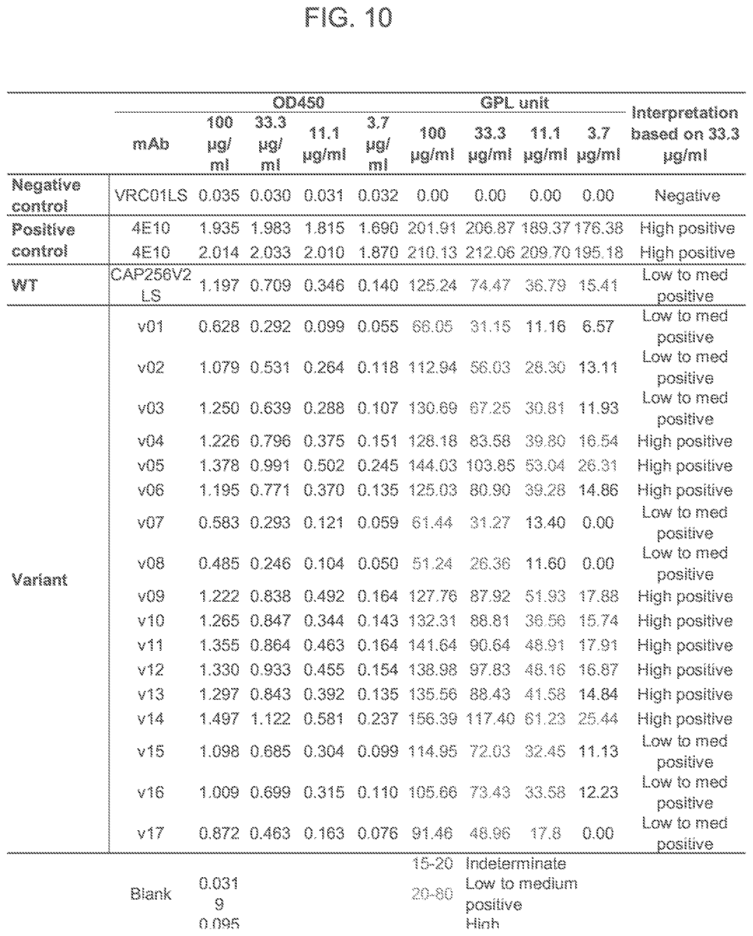

The 17 charge variants listed in the above table were produced and assessed for neutralization of a panel of four HIV-1 Env strains, as well as for autoreactivity and heparin chromatography. Several variants showed similar IC₈₀ to that of CAP256V2LS, with lower retention volume as measured by heparin affinity chromatography (FIGS. 4F, 9A-9B), and improved pharmnacokinetics in FcRn mouse, with half-life predicted to be similar or perhaps even longer than CAP256V2LS.

Based on these results, additional charge variants were designed that include combinations of the mutations described above, as shown in Table 6.

TABLE 6
Variant CAP256V2LS-J3-3 bispecific antibodies

| | | Light chain with mutations |
|---|---|---|
| | | based on CAP256V2LS-J3-3 LC, |
| Antibody name | Heavy Chain | SEQ ID NO: 15 |
| CAP256V2LS-J3-3.C1 | CAP256V2LS HC | R19E/K75E/R105Q |
| | SEQ ID NO: 14 | (SEQ ID NO: 18) |
| CAP256V2LS-J3-3.C2 | CAP256V2LS HC | R19E/K83E/R105N |
| | SEQ ID NO: 14 | (SEQ ID NO: 19) |
| CAP256V2LS-J3-3.C3 | CAP256V2LS HC | R19E/K75E/K83E/R105Q |
| | SEQ ID NO: 14 | (SEQ ID NO: 20) |
| CAP256V2LS-J3-3.C4 | CAP256V2LS HC | R19E/K75E/K83E/R105N |
| | SEQ ID NO: 14 | (SEQ ID NO: 21) |
| CAP256V2LS-J3-3.C5 | CAP256V2LS HC | R19E/K62E/K75E/K83E/R105Q |
| | SEQ ID NO: 14 | (SEQ ID NO: 24) |
| CAP256V2LS-J3-3.C6 | CAP256V2LS HC | R19E/K62E/K75E/K83E/R105N |
| | SEQ ID NO: 14 | (SEQ ID NO: 25) |
| CAP256V2LS-J3-3.C7 | CAP256V2LS HC | K75E/K83E/R105Q |
| | SEQ ID NO: 14 | (SEQ ID NO: 22) |
| CAP256V2LS-J3-3.C8 | CAP256V2LS HC | R19E/K75E/K83E |
| | SEQ ID NO: 14 | (SEQ ID NO: 23) |

The charge variants listed in Table 6 were produced and assessed for neutralization of a panel of four HIV-1 Env strains (FIG. 4F). These results show that several of the charge variants, including CAP256.J3LS and VRC26.25L-J3-3.C2 have similar neutralization properties and substantially the same or longer half-life as the CAP256V2LS antibody.

The best variant, named CAP256.J3LS, had mutations in J3 of R19E, K83E, and R105Q, and low autoreactivity, good neutralization IC₈₀s, and decent pharmacokinetics in FcRn-KI mice (FIGS. 4G,10). The amino acid sequences of the CAP256.J3LS V_{H} and VL (with VL fused to J3) are provided as SEQ ID NOs: 14 and 17, respectively. As discussed above, CAP256.J3LS includes a CAP256V2LS based binding domain including heavy chain variable region with HCDR1, HCDR2, and HCDR3 sequences set forth as SEQ ID NOs: 10, 11, and 12, respectively, and a CAP256V2LS light chain variable region with LCDR1, LCDR2, and LCDR3 sequences set forth as SEQ ID NOs: 6, 7, and 8, respectively. However, as noted above, the CAP256V2LS light chain variable region is not involved with antigen binding. CAP256.J3LS also includes a modified J3 V_{H}H fused the n-terminus of the CAP256V2LS light chain via a 15aa flexible peptide linker (SEQ ID NO: 16). The modified J3 V_{H}H includes R19E, K83E, and R105Q substitutions and has an amino acid sequence set forth as SEQ ID NO: 26.

### CAP256.J3LS Properties

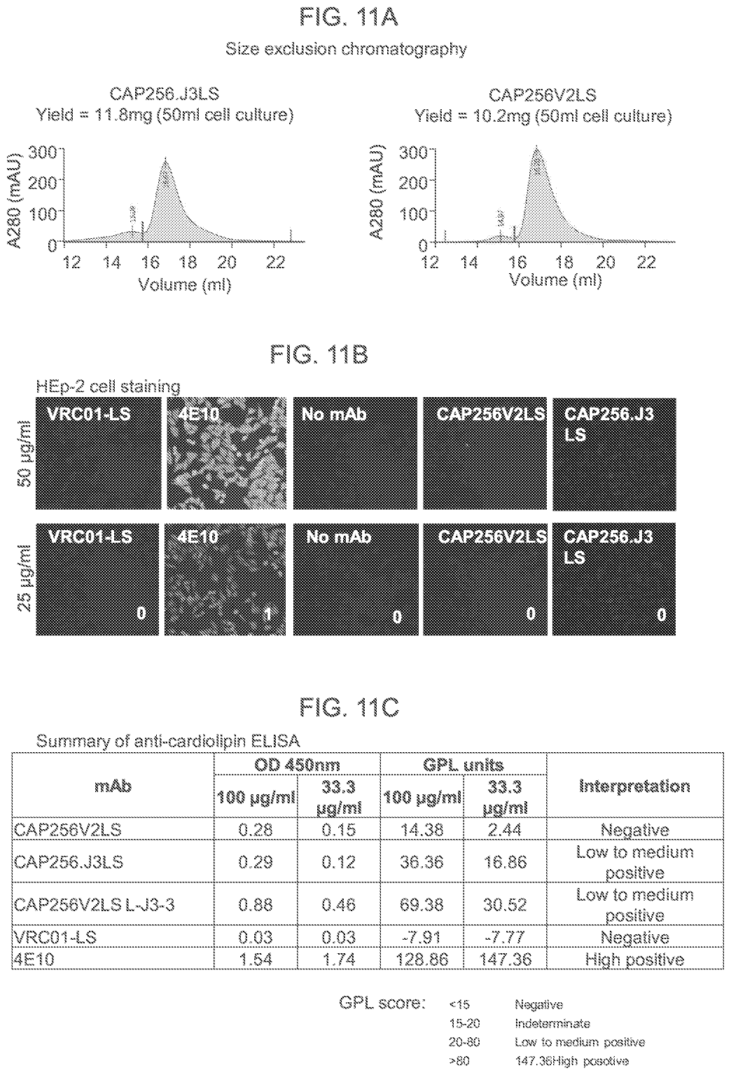

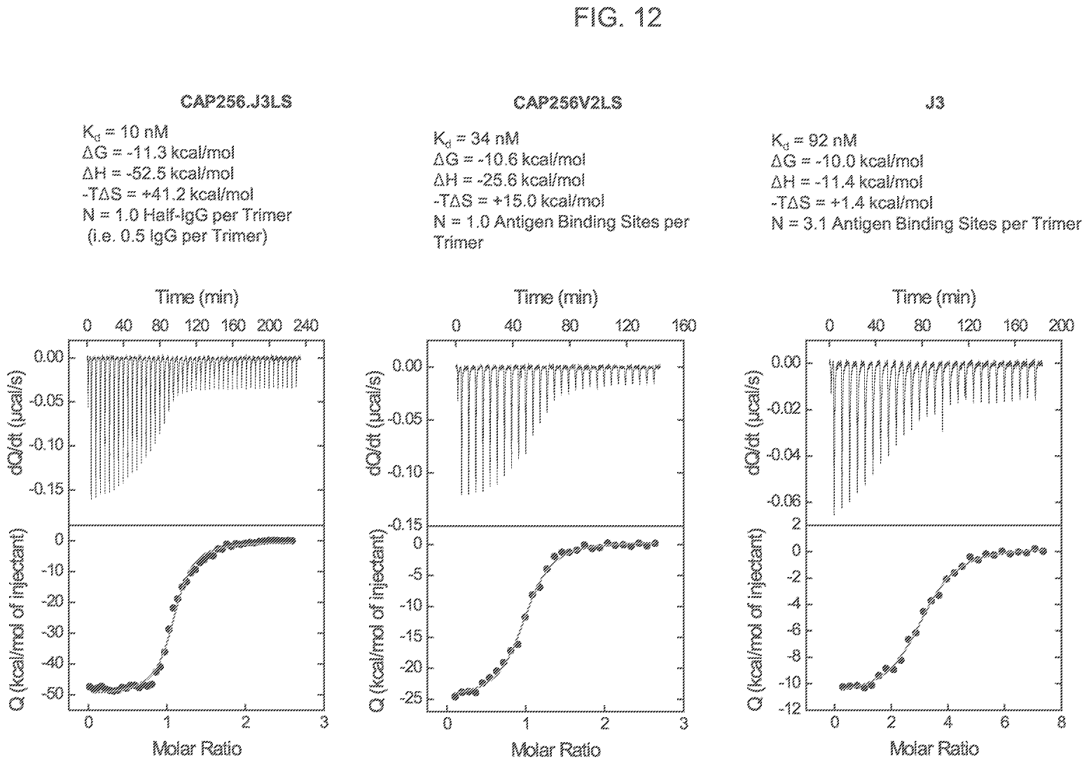

The CAP256.J3LS bispecific antibody expressed well in mammalian cells (FIG. 11) and could be purified following the same steps as its parent antibody CAP256V2LS, as the additional J3 component was only attached to the light chain. Isothermal titration calorimetry (ITC) with BG505 DS-SOSIP.664 showed improved affinity for CAP256.J3LS relative to CAP256V2LS and J3 (FIG. 12).

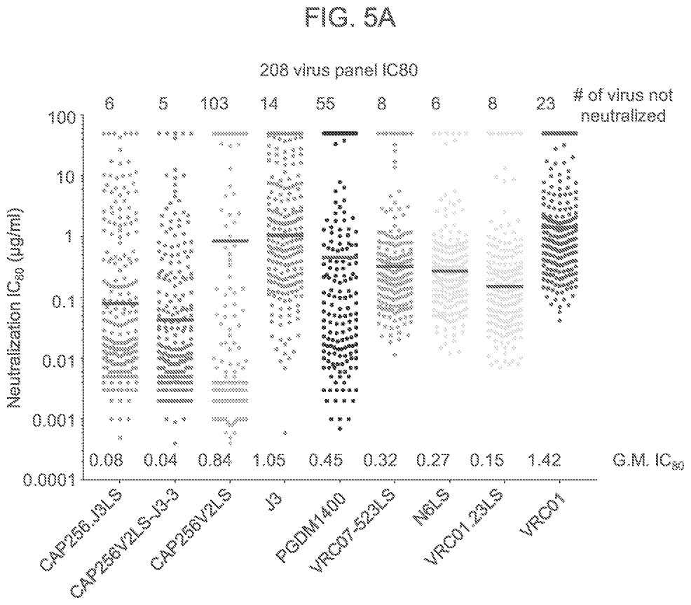

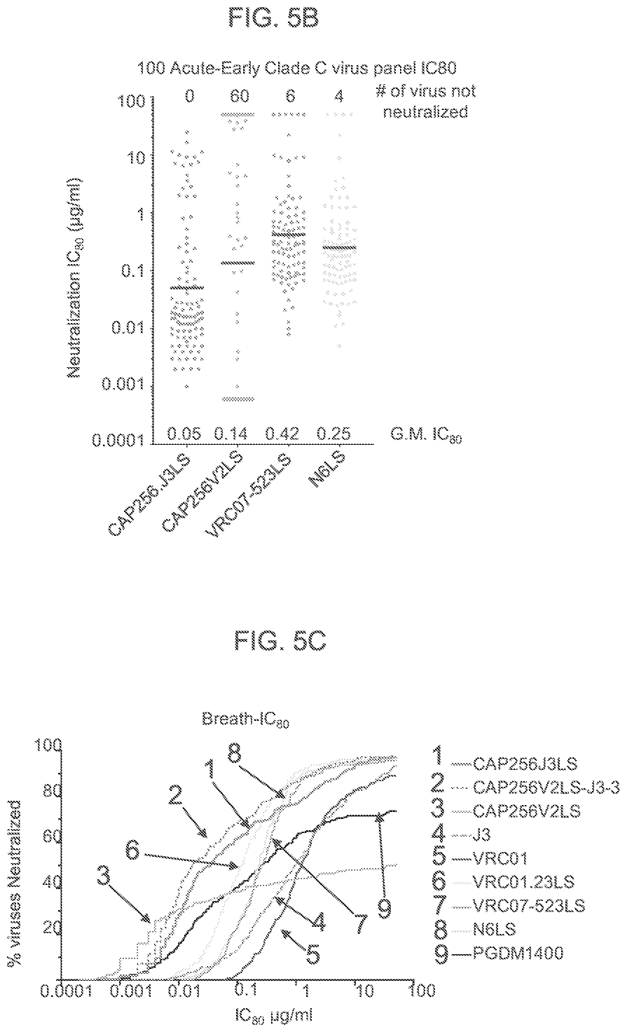

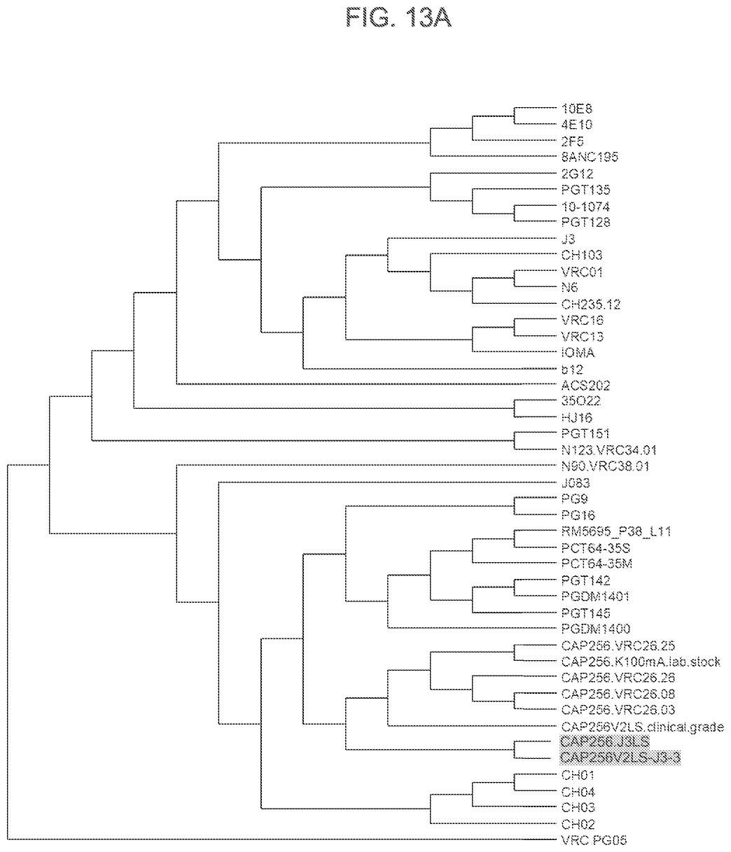

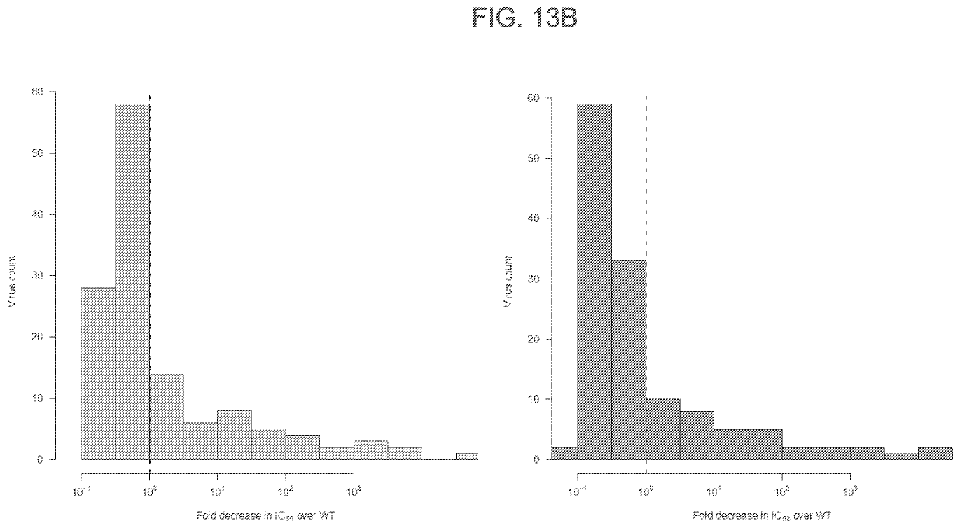

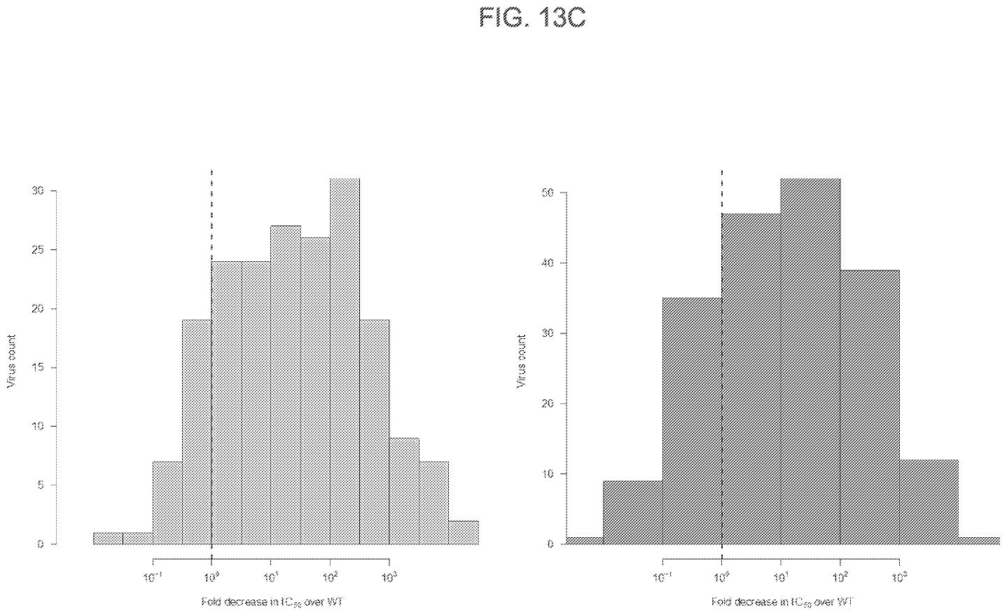

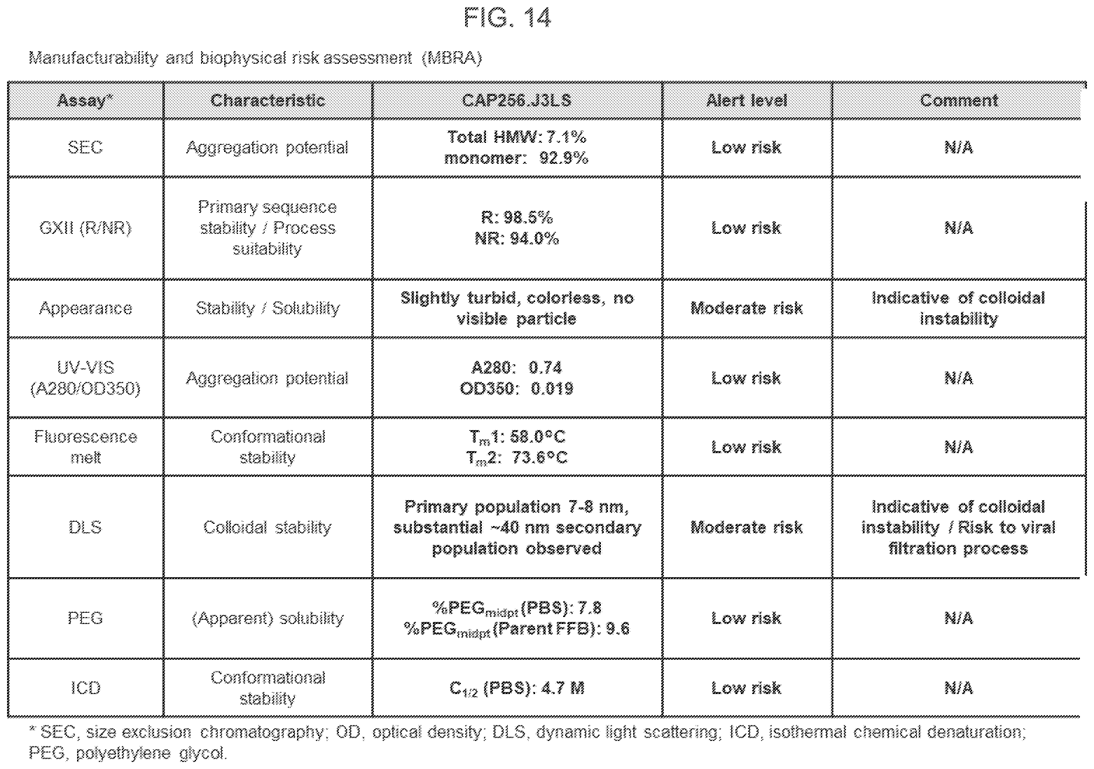

We assessed the neutralization on a 208-strain panel (FIG. 5A) and a 100-strain clade-C-specific panel (FIG. 5B). On the 208-strain panel, CAP256.J3LS showed neutralization potencies that were substantially superior to other broad neutralizers, including PGDM1400 and N6LS, neutralizing 97% with a geometric mean IC₈₀ of 0.08 μg/ml (FIG. 5A; Table 7). Interestingly, the analysis of the neutralization specificity showed the V2-apex directed neutralization to dominate, with the neutralization specificity of the CAP256-J3 variants clustering closer to CAP256-VRC26.25 than to J3 (FIG. 13). This clustering is likely a consequence of the neutralization specificity dependency on the rank order of neutralization, as the CAP256-VRC26.25 parent is more potent though less broad than the J3 parent. On the Seaman clade-C panel, 100% of the 100 clade C strains were neutralized (FIG. 5B and Table 8). CAP256.J3LS exhibited a geometric mean IC₈₀ of 0.05 μg/ml, substantially better than those of CAP256V2LS (0.14 μg/ml), VRC07-523LS (0.42 μg/ml), and N6LS (0.25 μg/ml). Finally, we carried out a manufacturability and biophysical risk assessment, and found CAP256.J3LS to be mostly low risk, except for slight colloidal instability observed at high concentration or with dynamic light scattering (DLS) (FIG. 14).

TABLE 7
Summary of neutralization activity on a 208-virus panel.

| | CAP256.J3LS | CAP256V2LS-J3-3 | CAP256V2LS | J3 |
|---|---|---|---|---|
| # Viruses | 208 | 208 | 208 | 208 |

Total VS neutralized

| | | | | |
|---|---|---|---|---|
| IC50 <50 μg/ml | 204 | 205 | 131 | 198 |
| IC50 <10 μg/ml | 202 | 204 | 126 | 190 |
| IC50 <1.0 μg/ml | 183 | 196 | 120 | 144 |
| IC50 <0.1 μg/ml | 150 | 167 | 109 | 68 |
| IC50 <0.01 μg/ml | 108 | 122 | 95 | 15 |

% VS neutralized

| | | | | |
|---|---|---|---|---|
| IC50 <50 μg/ml | 98 | 99 | 63 | 95 |
| IC50 <10 μg/ml | 97 | 98 | 61 | 91 |
| IC50 <1.0 μg/ml | 88 | 94 | 58 | 69 |
| IC50 <0.1 μg/ml | 72 | 80 | 52 | 33 |
| IC50 <0.01 μg/ml | 52 | 59 | 46 | 7 |
| Median IC50 | 0.0090 | 0.0050 | 0.0420 | 0.2955 |
| Geometric mean | 0.0228 | 0.0120 | 0.1473 | 0.3302 |

Total VS neutralized

| | | | | |
|---|---|---|---|---|
| IC80 <50 μg/ml | 202 | 203 | 105 | 194 |
| IC80 <10 μg/ml | 192 | 199 | 100 | 170 |
| IC80 <1.0 μg/ml | 160 | 179 | 91 | 104 |
| IC80 <0.1 μg/ml | 129 | 142 | 80 | 27 |
| IC80 <0.01 μg/ml | 56 | 75 | 64 | 3 |

% VS neutralized

| | | | | |
|---|---|---|---|---|
| IC80 <50 μg/ml | 97 | 98 | 50 | 93 |
| IC80 <10 μg/ml | 92 | 96 | 48 | 82 |
| IC80 <1.0 μg/ml | 77 | 86 | 44 | 50 |
| IC80 <0.1 μg/ml | 62 | 68 | 38 | 13 |
| IC80 <0.01 μg/ml | 27 | 36 | 31 | 1 |
| Median IC80 | 0.0350 | 0.0170 | 0.0040 | 0.9790 |
| Geometric mean | 0.0787 | 0.0427 | 0.0154 | 1.0507 |

TABLE 8
Summary of neutralization activity on an acute-early clade C panel.

| IC50 | CAP256.J3LS | CAP256V2LS | VRC07-523-LS | N6LS |
|---|---|---|---|---|
| # Viruses* | 100 | 100 | 100 | 100 |

Strains neutralized (%)

| | | | | |
|---|---|---|---|---|
| IC50 <50 μg/ml | 100 | 76 | 95 | 97 |
| IC50 <10 μg/ml | 100 | 73 | 95 | 96 |
| IC50 <1.0 μg/ml | 93 | 70 | 92 | 94 |
| IC50 <0.1 μg/ml | 79 | 67 | 60 | 68 |

For sensitive viruses only:

| | | | | |
|---|---|---|---|---|
| Median IC50 | 0.005 | 0.001 | 0.067 | 0.059 |
| Geometric mean | 0.010 | 0.002 | 0.064 | 0.063 |

For all viruses:

| | | | | |
|---|---|---|---|---|
| Median IC50 | 0.005 | 0.001 | 0.073 | 0.060 |
| Geometric mean | 0.010 | 0.025 | 0.090 | 0.072 |

| IC80 | CAP256J3LS | CAP256.25V2LS | VRC07-523-LS | N6 |
|---|---|---|---|---|
| # Viruses* | 100 | 100 | 100 | 100 |

Strains neutralized^{&} (%)

| | | | | |
|---|---|---|---|---|
| IC80 <50 μg/ml | 100 | 70 | 94 | 96 |
| IC80 <10 μg/ml | 96 | 64 | 93 | 95 |
| IC80 <1.0 μg/ml | 82 | 58 | 75 | 82 |
| IC80 <0.1 μg/ml | 71 | 49 | 24 | 30 |

For sensitive viruses only:

| | | | | |
|---|---|---|---|---|
| Median IC80 | 0.019 | 0.001 | 0.291 | 0.207 |
| Geometric mean | 0.050 | 0.011 | 0.307 | 0.199 |

For all viruses:

| | | | | |
|---|---|---|---|---|
| Median IC80 | 0.019 | 0.188 | 0.312 | 0.222 |
| Geometric mean | 0.050 | 0.135 | 0.417 | 0.248 |

### CONCLUSION

In this study, we created a bispecific antibody by linking the light chain of CAP256-VRC26.25 with the J3 nanobody and optimizing half-life to create CAP256.J3LS, a variant with improved breadth, potency, and half-life. One of the broadest and most potent V2 neutralizers, PGDM1400 (Julg et al. Sci Transl Med. 2017;9(406):eaall321), neutralizes 78% of the 208-strain panel with IC₈₀<50 μg/ml at a geometric mean IC₈₀ of 0.069 μg/ml, whereas CAP256.J3LS neutralized with a breadth of 97% with a geometric mean IC₈₀ of 0.035 μg/ml. The bispecific antibody BISC-1C (Davis-Gardner et al., mBio. 2020;11:e03080-19), which combines CAP256-VRC26.25 with PGT128, neutralizes all 15 strains tested with an IC₈₀ of 0.032 μg/ml, whereas CAP256.J3LS neutralized these 15 strains with an IC₈₀ of 0.026 μg/ml and neutralized 10 of the 15 strains more potently.

Bispecific antibodies can face manufacturing issues arising from their having two different heavy chain-light chain pairings. A nanobody-antibody bispecific, made by appending the light chain to the C terminus of the nanobody, would bypass these issues because the bispecific antibody would remain symmetric, with both arms having light chain-linked nanobodies. Here, we show that such a bispecific nanobody-antibody can be produced with high yield and, further, can show substantial neutralization synergy, neutralizing better than both antibody and nanobody parental components for over half the 208 viruses tested.

### Methods

### Antibody Expression and Purification

Light chain expression constructs of CAP256V2LS and PGDM1400 antibody variants were synthesized (Gene Universal Inc.) and cloned into pVRC8400 expression vector. For the antibody production, 0.15 mL of Turbo293 transfection reagent (Speed BioSystems) were mixed into 2.5 mL Opti-MEM medium (Life Technology) and incubated for 5 min at room temperature (RT). 50 g of plasmid DNAs (25 g heavy chain and 25 g of light chain) were mixed into 2.5 mL of Opti-MEM medium in another tube. The diluted transfection reagent was added into Opti-MEM medium containing plasmid DNAs. Transfection reagents and DNA mixtures were incubated for 15 min at RT and added to 40 mL of Expi293 cells (Life Technology) at 2.5 million cells/ml. The transfected cells were cultured in a shaker incubator at 120 rpm, 37° C., 9% CO₂ for 5 days. Antibodies in clarified supernatants were purified over 0.5 mL Protein A (GE Health Science) resin in columns. Antibodies were eluted from Protein A columns with a low pH immunoglobulin G (IgG) elution buffer (Pierce) and immediately neutralized with one-tenth volume of 1 M Tris-HCL pH 8.0. The antibodies were buffer exchanged in phosphate-buffered saline (PBS) by dialysis and then the concentration was adjusted to 0.5 mg/ml and filtered (0.22 m) for neutralization assays. For size-exclusion chromatography analysis, 0.5 mg of 1 mg/ml antibody was injected onto the Superose 6 Increase column (Cytiva Life Sciences) on a BioRad (Hercules, CA) NGC Chromatography System Quest 10. The flow rate was set to 0.5 mL/min, and the mobile phase B was 1× PBS (Thermo Fisher).

### Expression and Purification of HIV-1 Env Trimer

A non-tagged HIV Env trimer from the isolate CAP256.wk34.c80 (GenBank: KT698226.1) was stabilized and produced in transiently transfected 293 F cells as previously described (Sanders et al. PLoS Pathog. 2013;9(9):e1003618). Briefly, 600 g of the plasmid encoding the trimer and 150 g the plasmid encoding human furin were mixed and used to transfect 1 L of 293 F cells using Turbo293 transfection reagent (Speed BioSystems). Cells were incubated in shakers at 120 rpm, 37° C., and 9% C02. On the next day, 80 ml HyClone SFM4HEK293 medium and 20 ml FreeStyle™ 293 Expression Medium were added to each liter of cells. The native-like Env trimer protein was purified from the supernatant harvested on day 7 by 2G12 affinity chromatography, followed by gel filtration on a Sephadex200 16/60HL column in PBS.

### Anti-HIV-1 ENV Trimer ELISA

Twenty-four hours prior to the DNA-transient transfection, 100 μl/well of log-phase growing HEK 293 T cells were seeded into a flat bottom 96-well tissue culture plate (Coming) at a density of 3×10⁵ cells/ml in an optimized expression medium (RealFect-Medium, ABI Scientific) and incubated at 37° C., 5% CO₂ for 24 hours. Prior to transfection, 40 μl/well of spent medium was removed. For transient transfection, 0.15 μg of heavy-chain plasmid DNA was mixed with 0.15 μg of light-chain plasmid DNA in Opti-MEM medium (Invitrogen), and final volume 10 μl per well in a round bottom 96-well plate, followed by mixing with 10 μl/well of 0.9 μl TrueFect-Max transfection reagent (United BioSystems) in Opti-MEM medium. After incubation for 15 min at RT, 20 μl/well of DNA-TrueFect-Max complex was mixed with growing cells in the 96-well tissue culture plate and incubated at 37° C., 5% C02. At 20 hours post transfection, each well of culture was fed with 30 μl/well of enriched expression medium (CelBooster Cell Growth Enhancer Medium for Adherent Cell, ABI Scientific). After 5 days of transfection, the antibodies in supernatants in the 96-well tissue culture plate were characterized by 96-well-formatted ELISA. Briefly, 96-well ELISA plates (Nunc Maxisorp, Thermo Fisher Scientific) were coated overnight at 4° C. with 100 μl/well of 5 μg/ml lectin (Galanthus nivalis, Sigma-Aldrich) in 1× PBS. Between each subsequent step, plates were washed five times with PBS-T (PBS plus 0.05% Tween 20). After being coated, various HIV-1 trimer proteins were captured onto lectin-coated 96-well ELISA plates, respectively, by an incubation of 100 μl/well of 5 μg/ml each trimer protein for 2 hours at RT followed by blocking with 200 μl/well of CelBooster Cell Growth Enhancer Medium for Adherent Cell for 1 hour at RT. After plate wash, 30 μl/well of the expressed antibody supernatant mixed with 70 μl/well of PBS was incubated for 1 hour at RT, then followed by incubation for 30 min at RT with 100 μl/well of horseradish peroxidase (HRP)-conjugated goat anti-human IgG antibody (Jackson ImmunoResearch Laboratories Inc., PA, Cat. No.: 109-035-088), diluted at 1:10,000 in CelBooster Cell Growth Enhancer Medium for Adherent Cell plus 0.02% Tween 20. Finally, the reaction signal was developed with 100 μl/well of tetramethylbenzidine substrate (BioFX-TMB, SurModics) for 10 min at RT before the addition of 100 μl/well of 0.5 N sulfuric acid (Fisher Chemical) to stop the reaction. Plates were read at 450 nm wavelength (SpectraMax using SoftMax Pro, version 5, software; Molecular Devices, Sunnyvale, CA), and the optical densities (OD) were analyzed following subtraction of the nonspecific horseradish peroxidase background activity. All samples were measured in duplicate.

### Virus Neutralization Assay

As described below, neutralization was assessed in one of four formats of the Env-pseudotyped assay (Sarzotti-Kelsoe et al. J Immunol Methods. 2014; 409:131-46), all of which yielded highly similar results.
(1) Standard neutralization assays were performed in 96-well formats as follows: 10 μl of five-fold serially diluted mAbs in cDMEM was incubated with 40ul of diluted HIV-1 Env-pseudotyped virus and incubated for 30 minutes at 37° C. in a 96-well CulturPlate (Perkin Elmer). 20 μl of TZM-bl cells (10,000 cells/well) with or without 70 μg/ml DEAE-Dextran was then added and incubated overnight at 37° C. Each experiment plate also had a column of cells only (no antibody or virus) and a column of virus only (no antibody) as controls for background TZM-bl luciferase activity and maximal viral entry, respectively. Serial dilutions were performed with a change of tips at each dilution step to prevent carryover (Chuang et al., Structure. 2019; 27(1):196-206 e6). The following day, all wells received 100 μl of fresh cDMEM and were incubated overnight at 37° C. The following day, 50 μl of Steadylite Plus Reporter Gene Assay System (PerkinElmer) was added to all wells, and plates were shaken at 600RPM for 15 minutes. Luminometry was then performed on a SpectraMax L (Molecular Devices) luminometer. Percent neutralization is determined by calculating the difference in average Relative Light Units (RLU) between virus only wells (cells+virus column) and test wells (cells+plasma/Ab sample+virus), dividing this result by the average RLU of virus only wells (cell+virus column) and multiplying by 100. Background is subtracted from all test wells using the average RLU from the uninfected control wells (cells only column) before calculating the percent neutralization. Neutralizing plasma antibody titers are expressed as the antibody concentration required to achieve 50% neutralization and calculated using a dose-response curve fit with a 5-parameter nonlinear function.
(2) High-throughput screening was performed by single-point assay: supernatants from cells transfected in 96-well plates were used undiluted in the assay described above, in duplicate. Wells with >50% reduction in signal compared to baseline were scored as positive for neutralization.
(3) Selected monoclonal antibodies (mAbs) were assessed on a panel of 208 geographically and genetically diverse Env pseudoviruses representing the major subtypes and circulating recombinant forms (Kong et al. Science. 2016; 352(6287):828-33). Assays were performed by microneutralization in an optimized and qualified automated 384-well format as described (Sarzotti-Kelsoe et al. JImmunol Methods. 2014; 409:131-46), with the modification of changing tips after each antibody dilution. Data were analyzed as above.
(4) Select mAbs were assessed on a panel of 100 clade C Env pseudoviruses from acute infection (Wagh et al. PLoS Pathog. 2016;12(3):e1005520). Neutralization assays were conducted using TZM.bl cells as previously described (Sarzotti-Kelsoe et al. JImmunol Methods. 2014; 409:131-46). Briefly, mAb samples were tested in duplicate in 96-well plates using a primary concentration of 50 μg/ml or 1 μg/ml and serially diluted fivefold seven times. The 1 μg/ml start value was used instead of changing tips, as noted above. The HIV-1 Env pseudovirus was added to antibody serial dilutions and plates were incubated for 1 h at 37° C. TZM.bl cells were then added at 1×10⁴ μl/well with DEAE-Dextran at a final concentration of 11 μg/ml. After 48-h incubation at 37° C., plates were harvested using Bright-Glo luciferase (Promega) and luminescence detected using a GloMax Navigator luminometer (Promega, Madison, WI). Antibody concentrations that inhibited 50% or 80% of viral infection were determined (IC50 and IC80 titers, respectively). Neutralization assays were conducted in a laboratory meeting Good Clinical Laboratory Practice quality assurance criteria.

### Antibody Heparin Affinity Chromatography

Each antibody sample was diluted in 1500 μL of mobile phase A (MPA), 10 mM sodium phosphate, pH 7.2±0.2 to a final concentration of approximately 20 μg/mL. It was then injected onto the HiTrap 1 mL Heparin HP column (Cytiva Life Sciences) on a BioRad (Hercules, CA) NGC Chromatography System Quest 10. The flow rate was set to 1.0 mL/min, and the mobile phase B (MPB) was 10 mM sodium phosphate, 1 M NaCl, pH 7.2±0.2. The column was equilibrated in 100% MPA before each injection; the gradient was as follows (1): 0-2 min, 100% MPA; (2): 2-12 min, 100% MPA to 100% MPB; (3) 12-14 min, 100% MPB. UV absorbance was detected at 280 nm using Chromlab.

### Autoreactivity Analysis of Antibodies

Autoreactivity of antibodies was evaluated using the ANA Hep-2 Test System (ZEUS Scientific, Cat. No: FA2400) and anticardiolipin ELISA (Inova Diagnostics Cat. No.: 708625). Briefly, all antibodies were tested at 25 and 50 μg/ml as per the protocol from the manufacturer of the ANA Hep-2 Test System. Antibodies VRC01LS, 4E10, VRC07-523-LS, and VRC07-G54W were used as controls and scored as 0, 1, 2, and 3, respectively. HEp-2 cells were obtained from ZEUS Scientific. Slides were imaged on a Nikon Eclipse Ts2R microscope with a 20× objective lens for 500 ms. The fluorescent signals of the test antibodies were estimated visually in comparison to the control ones. Scores over 1 at 25 μg/ml were defined as autoreactive, and between 0 and 1 as mildly autoreactive. In the cardiolipin ELISA, all the antibodies were tested at 100 μg/ml, followed by a 3-fold serial dilution. IgG phospholipid (GPL) units were derived from the standard curve. GPL score below 20 was considered as not reactive, and between 20 and 80 as low positive and greater than 80 as high positive. The reported results are representative of at least two independent experiments.

### Isothermal Titration Calorimetry Measurement

Binding experiments by ITC were performed at 37° C. using a MicroCal VP-ITC microcalorimeter from Malvern Panalytical (Northampton, MA, USA). All reagents were dissolved in and exhaustively dialyzed against PBS, pH 7.4. The syringe was filled with either J3 nanobody at a concentration of ˜0.3 mg/mL (˜24 μM) or either CAP256V2LS IgG or bispecific CAP256.J3LS IgG at ˜1.0 mg/mL (˜6 μM IgG). The calorimetric cell was filled with BG505 DS-SOSIP.664 Env trimer at a concentration of ˜0.2 mg/mL (˜1 μM trimer) and either an IgG or a nanobody was added stepwise in 6 or 10 μL aliquots with 300 s interval during a continuous stirring at 300 rpm. The heat evolved upon each injection was obtained from the integral of the calorimetric signal, and the heat associated with binding was obtained after subtraction of the heat of dilution. The enthalpy change, ΔH, the association constant, Ka (the dissociation constant, Kd=1/Ka) and the stoichiometry, N, were obtained by nonlinear regression of the data to a single-site binding model using Origin with a fitting function made inhouse. The Gibbs energy, ΔG, was calculated from the binding affinity using ΔG=−RTlnKa (R=1.987 cal/(K×mol)) and T is the absolute temperature in kelvin. The entropy contribution to the Gibbs energy, −TΔS, was calculated from the relation ΔG=ΔH−TΔS. The results were normalized per mole of nanobody or half-IgG and the stoichiometry, N, denotes the number of half-IgG per trimer.

### Pharmacokinetic Study in Human Neonatal Fc Receptor (FcRn) Transgenic Mice

Human FcRn transgenic mice (FcRn−/− hFcRn (32) Tg mice, JAX stock #014565, The Jackson Laboratory) were used to assess the pharmacokinetics of wild type and Fv glycan-removed antibodies. Each animal was infused intravenously with 5 mg mAb/kg of body weight. Whole blood samples were collected at day 1, 2, 5, 7, 9, 14, and 21. Serum was separated by centrifugation. Serum mAb levels were measured by ELISA using either anti-idiotypic antibodies (for VRC01 LS; CAP256-VRC26.25, CAP256V2LS, CAP256V2LS-J3-3, or CA256.J3LS) as described previously (Rudicell et al. J Virol. 2014; 88(21):12669-82). All mice were bred and maintained under pathogen-free conditions at the American Association for the Accreditation of Laboratory Animal Care-accredited Animal Facility at the National Institute of Allergy and Infectious Diseases and housed in accordance with the procedures outlined in the Guide for the Care and Use of Laboratory Animals. All the mice were between 6 and 13 weeks of age. The study protocol was evaluated and approved by the National Institutes of Health's Animal Care and Use Committee (ASP VRC-18-747).

### Neutralization Fingerprinting Analysis

The neutralization fingerprint of an mAb or polyclonal plasma is defined as the potency pattern with which the antibody/plasma neutralizes a set of diverse viral strains. The neutralization fingerprints of 46 mAbs, including all described CAP256.VRC26.25 variants, were compared and clustered according to fingerprint similarity, as described previously (Georgiev et al. Science. 2013; 340:751-56). A set of 208 HIV-1 strains was used in the neutralization fingerprinting analysis.

### Cryo-EM Data Collection and Processing

The BG505 DS-SOSIP.664 Env trimer (Gulla et al., Vaccine. 2021; 39(25):3379-87; Kwon et al., Nat Struct Mol Biol. 2015; 22(7):522-31) was incubated with a molar excess of CAP256V2LS-J3-3 bispecific antibody Fab fragments. A volume of 2.3 μl of the complex at 2 mg/ml concentration was deposited on a C-flat 1.2/1.3 grid (protochip.com) and vitrified with an FEI Vitrobot Mark IV with a wait time of 30 seconds, blot time of 3 seconds, and blot force of 1. Data collection was performed on a Titan Krios electron microscope with Leginon using a Gatan K3 direct detection device. Exposures were collected in movie mode for 2 seconds with the total dose of 51.19 e⁻/Å² fractionated over 50 raw frames. cryoSPARC v3.3 was used for frame alignment, CTF estimation, 2D classifications, ab initio 3D reconstruction, homogeneous refinement, and nonuniform 3D refinement. Initial 3D reconstruction and final refinements were performed using C1 symmetry. The final resolution of the C1 non-uniform refinement was 3.18 Å.

Coordinates from the PDB IDs 6VTT and 7LPN were used for initial fits to the reconstructed map. This was followed by simulated annealing and real space refinement in Phenix v1.20 with the sharpened map from cryoSPARC v3.3 and with a density modified map from Phenix Resolve and manually fit with Coot v0.9.8 and then improved through iterative rounds. Geometry and map fitting parameters were evaluated using Molprobity v4.5.1 and EMRinger. PyMOL v2.5 (pymol.org) and ChimeraX v1.3 were used to generate figures.

### Manufacturability Assessment

Manufacturability for CAP256.J3LS was assessed by visual inspection, dynamic light scattering, thermal transitions by dynamic light scattering, differential scanning calorimetry, circular dichroism, and isothermal chemical denaturation as previously described.

### Example 2

### Treatment of HIV-1 Using an HIV-1 Env Specific Bispecific Antibody

This example describes a particular method that can be used to treat HIV-1 infection in a human subject by administration of a disclosed HIV-1 Env-specific bispecific antibody. Although particular methods, dosages, and modes of administrations are provided, one skilled in the art will appreciate that variations can be made without substantially affecting the treatment.

Based upon the teaching disclosed herein, HIV-1 infection can be treated by administering a therapeutically effective amount of one or more of the neutralizing bispecific described herein, thereby reducing or eliminating HIV-1 infection.

### Screening Subjects

In particular examples, the subject is first screened to determine if they have an HIV-1 infection. Examples of methods that can be used to screen for HIV-1 infection include a combination of measuring a subject's CD4+ T cell count and the level of HIV-1 virus in serum blood levels. Additional methods using an HIV-1 Env-specific antibody described herein can also be used to screen for HIV-1 infection.

In some examples, HIV-1 testing consists of initial screening with an enzyme-linked immunosorbent assay (ELISA) to detect antibodies to HIV-1. Specimens with a nonreactive result from the initial ELISA are considered HIV-1-negative unless new exposure to an infected partner or partner of unknown HIV-1 status has occurred. Specimens with a reactive ELISA result are retested in duplicate. If the result of either duplicate test is reactive, the specimen is reported as repeatedly reactive and undergoes confirmatory testing with a more specific supplemental test (e.g., Western blot or an immunofluorescence assay (IFA)). Specimens that are repeatedly reactive by ELISA and positive by IFA or reactive by Western blot are considered HIV-positive and indicative of HIV-1 infection. Specimens that are repeatedly ELISA-reactive occasionally provide an indeterminate Western blot result, which may be either an incomplete antibody response to HIV-1 in an infected person, or nonspecific reactions in an uninfected person. IFA can be used to confirm infection in these ambiguous cases. In some instances, a second specimen will be collected more than a month later and retested for subjects with indeterminate Western blot results. In additional examples, nucleic acid testing (e.g., viral RNA or proviral DNA amplification method) can also help diagnosis in certain situations.

The detection of HIV-1 in a subject's blood is indicative that the subject is infected with HIV-1 and is a candidate for receiving the therapeutic compositions disclosed herein. Moreover, detection of a CD4+ T cell count below 350 per microliter, such as 200 cells per microliter, is also indicative that the subject is likely to have an HIV-1 infection.

Pre-screening is not required prior to administration of the therapeutic compositions disclosed herein

### Pre-Treatment of Subjects

In particular examples, the subject is treated prior to administration of a therapeutic agent that includes one or more antiretroviral therapies known to those of skill in the art. However, such pre-treatment is not always required, and can be determined by a skilled clinician.

### Administration of Therapeutic Compositions

Following subject selection, a therapeutically effective dose of a HIV-1 Env-specific bispecific antibody described herein is administered to the subject (such as an adult human or a newborn infant either at risk for contracting HIV-1 or known to be infected with HIV-1). Additional agents, such as anti-viral agents, can also be administered to the subject simultaneously or prior to or following administration of the disclosed agents. Administration can be achieved by any method known in the art, such as oral administration, inhalation, intravenous, intramuscular, intraperitoneal, or subcutaneous.

The amount of the composition administered to prevent, reduce, inhibit, and/or treat HIV-1 or a condition associated with it depends on the subject being treated, the severity of the disorder, and the manner of administration of the therapeutic composition. Ideally, an amount of the bispecific antibody that is sufficient to prevent, reduce, and/or inhibit, and/or treat the condition (e.g., HIV-1) in a subject without causing a substantial cytotoxic effect in the subject is administered. An effective amount can be readily determined by one skilled in the art, for example using routine trials establishing dose response curves. As such, these compositions may be formulated with an inert diluent or with a pharmaceutically acceptable carrier.

In one specific example, antibodies are administered at 5 mg per kg every two weeks or 10 mg per kg every two weeks. In another example, antibodies are administered at 50 μg per kg given twice a week for 2 to 3 weeks.

Administration of the therapeutic compositions can be taken long term (for example over a period of months or years).

### Assessment

Following the administration of one or more therapies, subjects with HIV-1 can be monitored for reductions in HIV-1 levels, increases in a subject's CD4+ T cell count, or reductions in one or more clinical symptoms associated with HIV-1 disease. In particular examples, subjects are analyzed one or more times, starting 7 days following treatment. Subjects can be monitored using any method known in the art. For example, biological samples from the subject, including blood, can be obtained and alterations in HIV-1 or CD4+ T cell levels evaluated.

### Additional Treatments

In particular examples, if subjects are stable or have a minor, mixed or partial response to treatment, they can be re-treated after re-evaluation with the same schedule and preparation of agents that they previously received for the desired amount of time, including the duration of a subject's lifetime. A partial response is a reduction, such as at least a 10%, at least 20%, at least 30%, at least 40%, at least 50%, or at least 70% in HIV-1 infection, HIV-1 replication or combination thereof. A partial response may also be an increase in CD4+ T cell count such as at least 350 T cells per microliter.

It will be apparent that the precise details of the methods or compositions described may be varied or modified without departing from the spirit of the described implementations. We claim all such modifications and variations that fall within the scope and spirit of the claims below.

1. A bispecific antibody, comprising:
a first binding domain comprising an antibody comprising a heavy chain variable region (V_{H}) comprising a heavy chain complementarity determining region (HCDR)1, a HCDR2, and a HCDR3 comprising amino acid sequences set forth as SEQ ID NOs: 2, 3, and 4, respectively, a light chain variable region (V_{L}), and a constant domain;
a second binding domain comprising a V_{H}H comprising a HCDR1, a HCDR2, and a HCDR3 comprising amino acid sequences set forth as SEQ ID NOs: 10, 11, and 12, respectively;
wherein the first binding domain specifically binds to a V1-V2 region of HIV-1 Envelope protein (Env), and the second binding domain specifically binds to a CD4 binding site of HIV-1 Env;
wherein the first and second binding domains can simultaneously bind to a single HIV-1 Env trimer;
wherein the C-terminus of the V_{H}H is fused to the N terminus of the V_{L} by a peptide linker; and
wherein the bispecific antibody neutralizes HIV-1.

2. The bispecific antibody of claim 1, wherein the V_{L} comprises a light chain complementarity determining region (LCDR)1, a LCDR2, and a LCDR3 comprising amino acid sequences set forth as SEQ ID NOs: 6, 7, and 8, respectively.

3. The bispecific antibody of claim 1, wherein
the V_{H} comprises the HCDR1, HCDR2, and HCDR3 set forth as SEQ ID NOs: 2, 3, and 4, respectively, and the remainder of the V_{H} is at least 90% identical to SEQ ID NO: 1;
the V_{L} comprises the LCDR1, LCDR2, and LCDR3 set forth as SEQ ID NOs: 6, 7, and 8, respectively, and the remainder of the V_{L} of the first binding domain is at least 90% identical to SEQ ID NO: 5, and/or
the V_{H}H comprises the HCDR1, HCDR2, and HCDR3 set forth as SEQ ID NOs: 10, 11, and 12, respectively, and the remainder of the V_{H}H of the second binding domain is at least 90% identical to SEQ ID NO: 9.

4. The bispecific antibody of claim 1, wherein the V_{L} comprises an amino acid sequence at least 90% identical to SEQ ID NO: 5.

5-6. (canceled)

7. The bispecific antibody of claim 1, wherein the peptide linker is from 5-30 amino acids in length, from 10⁻²⁰ amino acids in length, and/or is 15 amino acids in length.

8-9. (canceled)

10. The bispecific antibody of claim 1, wherein the peptide linker is a glycine-serine linker.

11. The bispecific antibody of claim 1, wherein the peptide linker comprises or consists of the amino acid sequence set forth as SEQ ID NO: 16.

12. The bispecific antibody of claim 1, wherein the V_{H}H comprises one or more amino acid substitutions in the framework region to replace positively charged amino acids with neutral or negatively charged amino acids.

13. The bispecific antibody of claim 1, wherein the V_{H}H comprises one or more of R19E, K75E, K83E, R10⁵N, R10⁵Q substitutions with reference to SEQ ID NO: 9.

14. The bispecific antibody of claim 13, wherein the V_{H}H comprises R19E, K83E, and R10⁵Q substitutions with reference to SEQ ID NO: 9.

15. The bispecific antibody of claim 1, wherein the V_{H} comprises or consists of the amino acid sequence set forth as SEQ ID NO: 1.

16. The bispecific antibody of claim 1, wherein the V_{L} comprises or consists of the amino acid sequence set forth as SEQ ID NO: 5.

17. The bispecific antibody of claim 1, wherein the V_{H}H comprises or consists of the amino acid sequence set forth as SEQ ID NO: 9 or amino acids 1-121 of any one of SEQ ID NOs: 17-23.

18. The bispecific antibody of claim 17, wherein the V_{H}H of the second binding domain comprises the amino acid sequence set forth as amino acids 1-121 of SEQ ID NO: 17.

19. The bispecific antibody of claim 1, wherein the V_{H} comprises the amino acid sequences set forth as SEQ ID NO: 1 and the V_{H}H fused to the V_{L} comprises the amino acid sequence set forth as amino acids 1-258 of SEQ ID NO: 17.

20. The bispecific antibody of claim 1, wherein the antibody is a recombinant IgG, IgM or IgA.

21. The bispecific antibody of claim 1, wherein the constant domain comprises a modification that increases binding to the neonatal Fc receptor.

22. The bispecific antibody of claim 21, wherein the constant domain is an IgG1 constant domain comprising M428L and N434S mutations.

23. The bispecific antibody of claim 22, wherein the heavy chain of the antibody comprises the amino acid sequence set forth as SEQ ID NO: 14 and the V_{H}H fused to the light chain of the antibody comprises the amino acid sequence set forth as any one of SEQ ID NOs: 15 or 17-23.

24. The bispecific antibody of claim 23, wherein the heavy chain of the antibody comprises the amino acid sequence set forth as SEQ ID NO: 14 and the V_{H}H fused to the light chain of the antibody comprises the amino acid sequence set forth as SEQ ID NO: 17.

25. The bispecific antibody of claim 1, linked to an effector molecule or a detectable marker.

26. The bispecific antibody of claim 25, wherein the detectable marker is a fluorescent, enzymatic, or radioactive marker.

27. A nucleic acid molecule encoding the bispecific antibody of claim 1.

28. The nucleic acid molecule of claim 27, operably linked to a promoter.

29. An expression vector comprising the nucleic acid molecule of claim 27.

30. A pharmaceutical composition, comprising:
a therapeutically effective amount of the bispecific antibody, nucleic acid molecule, or expression vector of claim 1; and
a pharmaceutically acceptable carrier.

31. A method of producing an antibody that specifically binds to HIV-1 Env, comprising:
expressing the nucleic acid molecule or expression vector of claim 27 in a host cell to produce the antibody in the host cell; and
purifying the antibody.

32. A method of detecting an HIV-1 infection in a subject, comprising:
contacting a biological sample from the subject with the bispecific antibody of claim 1 under conditions sufficient to form an immune complex; and
detecting the presence of the immune complex in the sample, wherein the presence of the immune complex in the sample indicates that the subject has the HIV-1 infection.

33. A method of inhibiting an HIV-1 infection in a subject, comprising administering to the subject an amount of the bispecific antibody, nucleic acid molecule, expression vector, or pharmaceutical composition of claim 1 effective to prevent or treat the HIV-1 infection in the subject.

34. The method of claim 33, wherein the subject is at risk of or has an HIV-1 infection.

35. (canceled)


SEQUENCE LISTING (55 sequences)

SEQ ID NO: 1 - protein, 145 aa, synthetic construct
  REGION 1..145: note = Recombinant antibody sequence
QVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGAQLPCAKSRGGLVGIADNWGQGTMVTVSS

SEQ ID NO: 2 - protein, 5 aa, synthetic construct
  REGION 1..5: note = Recombinant antibody sequence
GYGMH

SEQ ID NO: 3 - protein, 16 aa, synthetic construct
  REGION 1..16: note = Recombinant antibody sequence
SISHDGIKKYHAEKVW

SEQ ID NO: 4 - protein, 36 aa, synthetic construct
  REGION 1..36: note = Recombinant antibody sequence
DLREDECEEWWSDYYDFGAQLPCAKSRGGLVGIADN

SEQ ID NO: 5 - protein, 111 aa, synthetic construct
  REGION 1..111: note = Recombinant antibody sequence
QSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVL

SEQ ID NO: 6 - protein, 13 aa, synthetic construct
  REGION 1..13: note = Recombinant antibody sequence
SGNTSNIGNNFVS

SEQ ID NO: 7 - protein, 7 aa, synthetic construct
  REGION 1..7: note = Recombinant antibody sequence
ETDKRPS

SEQ ID NO: 8 - protein, 12 aa, synthetic construct
  REGION 1..12: note = Recombinant antibody sequence
ATWAASLSSARV

SEQ ID NO: 9 - protein, 121 aa, synthetic construct
  REGION 1..121: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSS

SEQ ID NO: 10 - protein, 5 aa, synthetic construct
  REGION 1..5: note = Recombinant antibody sequence
QYAMA

SEQ ID NO: 11 - protein, 14 aa, synthetic construct
  REGION 1..14: note = Recombinant antibody sequence
GMGAVPHYGEFVKG

SEQ ID NO: 12 - protein, 15 aa, synthetic construct
  REGION 1..15: note = Recombinant antibody sequence
SKSTYISYNSNGYDY

SEQ ID NO: 13 - protein, 258 aa, synthetic construct
  REGION 1..258: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTL

SEQ ID NO: 14 - protein, 475 aa, synthetic construct
  REGION 1..475: note = Recombinant antibody sequence
QVQLVESGGGVVQPGTSLRLSCAASQFRFDGYGMHWVRQAPGKGLEWVASISHDGIKKYHAEKVWGRFTISRDNSKNTLYLQMNSLRPEDTALYYCAKDLREDECEEWWSDYYDFGAQLPCAKSRGGLVGIADNWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 15 - protein, 353 aa, synthetic construct
  REGION 1..353: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 15 aa, synthetic construct
  REGION 1..15: note = Peptide linker sequence
GGSGGGGSGGGGSGG

SEQ ID NO: 17 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVKGRFTISRDNAKSTVYLQMSSLEPEDTAIYFCARSKSTYISYNSNGYDYWGQGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 18 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVKGRFTISRDNAESTVYLQMSSLKPEDTAIYFCARSKSTYISYNSNGYDYWGQGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 19 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVKGRFTISRDNAKSTVYLQMSSLEPEDTAIYFCARSKSTYISYNSNGYDYWGNGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 20 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVKGRFTISRDNAESTVYLQMSSLEPEDTAIYFCARSKSTYISYNSNGYDYWGQGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 21 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVKGRFTISRDNAESTVYLQMSSLEPEDTAIYFCARSKSTYISYNSNGYDYWGNGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 22 - protein, 353 aa, synthetic construct
  REGION 1..353: note = Recombinant antibody sequence
EVQLVESGGGLVQAGGFLRLSCELRGSIFNQYAMAWFRQAPGKEREFVAGMGAVPHYGEFVKGRFTISRDNAESTVYLQMSSLEPEDTAIYFCARSKSTYISYNSNGYDYWGQGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 23 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVKGRFTISRDNAESTVYLQMSSLEPEDTAIYFCARSKSTYISYNSNGYDYWGRGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 24 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVEGRFTISRDNAESTVYLQMSSLEPEDTAIYFCARSKSTYISYNSNGYDYWGQGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 25 - protein, 353 aa, synthetic construct
  REGION 1..353: note = Recombinant antibody sequence
EVQLVESGGGLVQAGGFLELSCELRGSIFNQYAMAWFRQAPGKEREFVAGMGAVPHYGEFVEGRFTISRDNAESTVYLQMSSLEPEDTAIYFCARSKSTYISYNSNGYDYWGNGTQVTVSSGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 26 - protein, 121 aa, synthetic construct
  REGION 1..121: note = Recombinant antibody sequence
EVQLVESGGGLVQAGGFLELSCELRGSIFNQYAMAWFRQAPGKEREFVAGMGAVPHYGEFVKGRFTISRDNAKSTVYLQMSSLEPEDTAIYFCARSKSTYISYNSNGYDYWGQGTQVTVSS

SEQ ID NO: 27 - protein, 343 aa, synthetic construct
  REGION 1..343: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 28 - protein, 348 aa, synthetic construct
  REGION 1..348: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 29 - protein, 343 aa, synthetic construct
  REGION 1..343: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 30 - protein, 349 aa, synthetic construct
  REGION 1..349: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSDKTHTGDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 31 - protein, 355 aa, synthetic construct
  REGION 1..355: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSDKTHTGDKTHTGDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 475 aa, synthetic construct
  REGION 1..475: note = Recombinant antibody sequence
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 33 - protein, 480 aa, synthetic construct
  REGION 1..480: note = Recombinant antibody sequence
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 34 - protein, 485 aa, synthetic construct
  REGION 1..485: note = Recombinant antibody sequence
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 35 - protein, 475 aa, synthetic construct
  REGION 1..475: note = Recombinant antibody sequence
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 36 - protein, 481 aa, synthetic construct
  REGION 1..481: note = Recombinant antibody sequence
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVDKTHTGDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 37 - protein, 487 aa, synthetic construct
  REGION 1..487: note = Recombinant antibody sequence
EVRLVESGGGLVKPGGSLRLSCSASGFDFDNAWMTWVRQPPGKGLEWVGRITGPGEGWSVDYAESVKGRFTISRDNTKNTLYLEMNNVRTEDTGYYFCARTGKYYDFWSGYPPGEEYFQDWGQGTLVIVSSGGGGSGGGGSGGGGSSELTQDPAVSVALKQTVTITCRGDSLRSHYASWYQKKPGQAPVLLFYGKNNRPSGIPDRFSGSASGNRASLTITGAQAEDEADYYCSSRDKSGSRLSVFGGGTKLTVDKTHTGDKTHTGDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 38 - protein, 455 aa, synthetic construct
  REGION 1..455: note = Recombinant antibody sequence
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 39 - protein, 460 aa, synthetic construct
  REGION 1..460: note = Recombinant antibody sequence
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 40 - protein, 465 aa, synthetic construct
  REGION 1..465: note = Recombinant antibody sequence
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVGGSGGGGSGGGGSGGQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 41 - protein, 455 aa, synthetic construct
  REGION 1..455: note = Recombinant antibody sequence
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 42 - protein, 460 aa, synthetic construct
  REGION 1..460: note = Recombinant antibody sequence
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDKTHTDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 43 - protein, 465 aa, synthetic construct
  REGION 1..465: note = Recombinant antibody sequence
QVQLVQSGGQMKKPGESMRISCRASGYEFIDCTLNWIRLAPGKRPEWMGWLKPRGGAVNYARPLQGRVTMTRDVYSDTAFLELRSLTVDDTAVYFCTRGKNCDYNWDFEHWGRGTPVIVSSGGGGSGGGGSGGGGSLTQSPGTLSLSPGETAIISCRTSQYGSLAWYQQRPGQAPRLVIYSGSTRAAGIPDRFSGSRWGPDYNLTISNLESGDFGVYYCQQYEFFGQGTKVQVDKTHTDKTHTDKTHTQSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 344 aa, synthetic construct
  REGION 1..344: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSGGSGGDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 45 - protein, 349 aa, synthetic construct
  REGION 1..349: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSGGSGGGGSGGDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 354 aa, synthetic construct
  REGION 1..354: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSGGSGGGGSGGGGSGGDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 344 aa, synthetic construct
  REGION 1..344: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSDKTHTDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 350 aa, synthetic construct
  REGION 1..350: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSDKTHTGDKTHTDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 356 aa, synthetic construct
  REGION 1..356: note = Recombinant antibody sequence
EVQLVESGGGLVQAGGFLRLSCELRGSIFNQYAMAWFRQAPGKEREFVAGMGAVPHYGEFVKGRFTISRDNAKSTVYLQMSSLKPEDTAIYFCARSKSTYISYNSNGYDYWGRGTQVTVSSDKTHTGDKTHTGDKTHTDFVLTQSPHSLSVTPGESASISCKSSHSLIHGDRNNYLAWYVQKPGRSPQLLIYLASSRASGVPDRFSGSGSDKDFTLKISRVETEDVGTYYCMQGRESPWTFGQGTKVDIKTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 50 - protein, 5 aa, synthetic construct
  REGION 1..5: note = Peptide linker sequence
GGSGG

SEQ ID NO: 51 - protein, 10 aa, synthetic construct
  REGION 1..10: note = Peptide linker sequence
GGSGGGGSGG

SEQ ID NO: 52 - protein, 5 aa, synthetic construct
  REGION 1..5: note = Peptide linker sequence
DKTHT

SEQ ID NO: 53 - protein, 11 aa, synthetic construct
  REGION 1..11: note = Peptide linker sequence
DKTHTGDKTHT

SEQ ID NO: 54 - protein, 17 aa, synthetic construct
  REGION 1..17: note = Peptide linker sequence
DKTHTGDKTHTGDKTHT

SEQ ID NO: 55 - protein, 217 aa, synthetic construct
  REGION 1..217: note = Recombinant antibody sequence
QSVLTQPPSVSAAPGQKVTISCSGNTSNIGNNFVSWYQQRPGRAPQLLIYETDKRPSGIPDRFSASKSGTSGTLAITGLQTGDEADYYCATWAASLSSARVFGTGTQVIVLGQPKVNPTVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS